(19) United States
(12) Patent Application Publication
(10) Pub. No.: US 2026/0274903 A1
(43) Pub. Date: Sep. 17, 2026

(54) OPTOGENETIC ACTIVATION FOR SIGNAL TRANSDUCTION

(71) Applicants: The Chinese University of Hong Kong, Hong Kong SAR (CN); The Hong Kong University of Science and Technology, Hong Kong SAR (CN)

(72) Inventors: Liting DUAN, Hong Kong SAR (CN); Kai LIU, Hong Kong SAR (CN); Chao YANG, Hong Kong SAR (CN); Peiyuan HUANG, Hong Kong SAR (CN)

(21) Appl. No.: 19/396,084

(22) Filed: Nov. 20, 2025

Related U.S. Application Data
(63) Continuation-in-part of application No. PCT/CN2024/095128, filed on May 24, 2024.
(60) Provisional application No. 63/469,144, filed on May 26, 2023.

(51) Int. Cl. C07K 14/415; C12N 9/12

(52) U.S. Cl. CPC C07K 14/415; C12N 9/12; C12Y 207/10001; C07K 2319/02; C07K 2319/03

(57) ABSTRACT

The present invention provides a recombinantly produced fusion protein that includes an optically responsive element fused to a downstream signaling element derived from an active domain of a signaling molecule, such as the intracellular domain of a receptor protein, and is therefore capable of triggering the signal transduction cascade by light exposure rather than the native activating event to the signaling molecule, such as the binding of a ligand to the receptor. The nucleic acid encoding the fusion protein and the host cells containing the fusion protein or the nucleic acid are also provided. Further provided are methods of using light exposure to achieve desirable downstream effects of a signal transduction pathway normally mediated by the signaling molecule, for example, in the promotion optic neuronal cell proliferation, survival, and/or regeneration.

### RELATED APPLICATIONS

This application is a continuation-in-part of International Patent Application No. PCT/CN2024/095128, filed May 24, 2024, which claims priority to U.S. Provisional Patent Application No. 63/469,144, filed May 26, 2023, the contents of all of the above are hereby incorporated by reference in the entirety for all purposes.

### SEQUENCE LISTING

A Sequence Listing conforming to the rules of WIPO Standard ST.26 is hereby incorporated by reference. Said Sequence Listing has been filed as an electronic document via PatentCenter encoded as XML in UTF-8 text. The electronic document, created on Mar. 25, 2026, is entitled “080015-1521215-039010US_ST26.xml”, and is 31,990 bytes in size.

### BACKGROUND OF THE INVENTION

Various signaling pathways are known to play critical roles in the regulation of cellular processes such as cell proliferation, differentiation, survival, migration, regeneration, etc. As these processes are directly relevant to many physiological states and pathological conditions, there exists a keen interest to effectively regulate and control pertinent cellular signal transduction pathways. One strategy to exert easy and efficient control of signal transduction is through the use of a genetically engineered upstream signaling molecule that directly responds to a light signal rather than the binding of an extracellular ligand. While a generic concept of this nature was previously disclosed in 2020 publication by Huang et al. (Journal of Molecular Biology 432:3761-3770), the present inventors have made significant improvement in this technology.

In mammals, retinal ganglion cells (RGCs) are the only output neurons that transmit visual signals from the eye to the brain. RGC death can lead to vision loss and blindness, which is the final pathway in many debilitating ocular diseases, such as retinal ischemia, diabetic retinopathy, and glaucoma. These eye diseases affect numerous patients and exert a substantial social and economic burden on the world. For example, glaucoma is a leading cause of blindness worldwide and will affect an estimated 112 million patients in 2040. While glaucoma causes worldwide 7% of all blindness, it leads to 23% of all blindness cases in Hong Kong because of population aging. Due to the increasing prevalence of glaucoma, the global market for its treatment is expected to rise from 2.6 billion in 2016 to 3.8 billion USD in 2026. Despite tremendous efforts to study the mechanisms and develop therapies for these eye diseases, no curative treatment for RGC death exists and the resulted vision loss cannot be restored. The present invention provides new methods for addressing the urgent needs of vision loss prevention and restoration.

### BRIEF SUMMARY OF THE INVENTION

In a first aspect, the present invention provides a fusion protein, which is recombinantly engineered to include at least two components: first, an optic-responsive element, and second, a downstream signaling element. The optic-responsive element is derived from the Arabidopsis thaliana cryptochrome 2 (CRY2) protein, for example, modified from its wild-type version, for example, by incorporating a Lysine to Serine (K→S) substitution at residue 2 of the protein (K2S), a deletion of the first two amino acids from the N-terminus of the protein (Δ1/2), a Glutamic acid to Alanine (E→A) substitution at residue 281 (E281A), or a Glutamic acid to Arginine (E→R) substitution at residue 490 (E490R, residue numbers all counting from the N-terminus), or any two, three, or all four of these mutations. In some embodiments, optic-responsive element comprises a truncated version of CRY2 protein, for example, the N-terminal segment of about 500 or 600 amino acids (e.g., the 1-498 segment) of the full-length CRY2 protein. The downstream signaling element is derived from the domain of a signaling molecule or a signal transducing protein responsible for activating downstream effector molecules, for example, the intracellular domain of a receptor protein that in its naturally occurring form has an extracellular domain to receive a ligand from outside of a cell, a transmembrane domain to anchor the receptor in the cell membrane, and an intracellular domain, which upon ligand binding to the extracellular domain undergoes structural changes (e.g., phosphorylation or dephosphorylation at one or more residues) and the changes in turn trigger a cascade of downstream events such as the activation or inactivation of various other proteins (e.g., enzymes such as kinases or phosphatases) involved in regulating cellular processes relating to the proliferation, regeneration, differentiation, death/survival, reorganization, secretion, migration, etc. of target cells and tissues. This fusion of the optic-responsive element and the downstream signaling element originated from two distinct proteins creates a hybrid protein that, instead of requiring an extracellular ligand to trigger a pertinent downstream signaling pathway for activating a desired cellular process, is now subject to rapid and easy activation by light exposure. Thus, virtually any receptor protein as well as some of their downstream effector molecules involved in the cellular signal transduction can contribute their respective domain responsible for downstream signaling for the recombinant engineering, production, and utilization of a corresponding, light-activable optogenetic hybrid protein.

As such, in some embodiments, the downstream signaling element of the fusion protein comprises the intracellular domain of a receptor protein that naturally mediates a cellular signaling pathway of interest, preferably with the extracellular and transmembrane domains both deleted. For example, the receptor may be a receptor tyrosine kinase (RTK), e.g., tropomyosin receptor kinase A/B/C (TrkA/B/C), epidermal growth factor receptor (EGFR), and fibroblast growth factor receptor (FGFR). In some embodiments, the downstream signaling element comprises a segment of TrkB, such as the intracellular domain of TrkB (iTrkB, e.g., the 455-822 segment of rat TrkB as shown in SEQ ID NO:2; or iTrkB in human TrkB protein, as shown in the underlined portion of SEQ ID NO:7). In other embodiments, the downstream signaling element of the fusion protein comprises a domain taken from and responsible for the activity of a signaling molecule for its role in a signal transduction pathway such as SOS/Ras signaling, AKT signaling, and GDNF signaling. For example, the kinase domain of an AKT1 protein (the 128-480 segment of the protein as depicted by Uniprot P31749 AKT1_HUMAN or P31750 AKT1_MOUSE) can be used as the downstream signaling element placed at the C-terminus of a fusion protein with a CRY2 mutant. Another example of a suitable downstream signaling element of the fusion protein of this invention is the catalytic domain of the signaling molecule known as the SOS protein (SOScat, the 550-1050 segment of the SOS protein).

In some embodiments, the downstream signaling element of the fusion protein comprises the intracellular domain of the TrkA or TrkB protein but without its extracellular and transmembrane domains. When present within a live host cell, the optic-responsive element of the fusion protein, a CRY2 mutant comprising one or more of mutations selected from Δ1/2, K2S, E281A, and E490R, upon exposure to a light within a pre-determined wavelength (e.g., a blue light of about 440 nm to about 490 nm wavelength) is able to cause structural or configurational changes in the fusion protein (e.g., oligomerization such as dimerization, phosphorylation at one or more Tyrosine residues, etc.), which in turn activates TrkA or TrkB downstream signal transduction pathway.

In some embodiments, the fusion protein has from the N-terminal the following components: optionally a membrane-targeting element, an optic-responsive element (e.g., a modified CRY2 protein), optionally a detectable moiety (e.g., mCherry or a fluorescent protein such as a green fluorescent protein or GFP), and an downstream signaling element (e.g., intracellular domain of TrkB, such as rat iTrkB or human iTrkB, shown as the underlined portions in SEQ ID NOs: 2 and 7, respectively). These components may be directly connected between two without any intervening extra amino acids or may be connected by one or more peptide linkers between adjacent components.

Detailed description of the Arabidopsis thaliana cryptochrome 2 (CRY2) protein as well as tropomyosin receptor kinase B (TrkB), including its extracellular domain, transmembrane domain, and intracellular domain, can be found in the literature, see, e.g., Huang et al., Journal of Molecular Biology (2020) 432, 3761-3770. Description of signaling molecules such as AKT1 and SOS as well as their kinase domain and catalytic domain can be found in various publications, see, e.g., Gureasko et al., (2008) Nature structural & molecular biology, 15(5), 452-461; Toettcher et al., (2013) Cell, 155(6), 1422-1434; Johnson et al., (2017) Developmental cell, 40(2), 185-192; Ong et al., (2016) PLOS One, 11(4), e0153487; and Katsura et al., (2015) Scientific reports, 5(1), 14589.

In some embodiments, a cell membrane-targeting element is also present in the fusion protein so as to direct the fusion protein to an intracellular location within a close proximity to the membrane of a host cell. For example, an N-terminal Lyn tag or a CAAX motif may be included in the fusion protein as the cell membrane-targeting element. In some embodiments, the fusion protein does not include any cell membrane-targeting element. In such instances, the localization of the fusion protein may be achieved by the use of a second fusion protein, which comprises a cell membrane-targeting element (e.g., an N-terminal Lyn tag or a CAAX motif) and a CRY2-binding element (e.g., Arabidopsis protein CIB1 or an N-terminal portion thereof comprising the 1-170 segment). This second fusion protein is therefore able to not only direct the first fusion protein to be near host cell membrane and thus accessible to a light signal but also cause dimerization of the first fusion protein upon light activation. In some embodiments, the second fusion protein has from the N-terminal the following components: a CRY2-binding element (e.g., Arabidopsis protein CIB1 or an N-terminal portion thereof), optionally a detectable moiety (e.g., mCherry or GFP), and a membrane-targeting element (e.g., an N-terminal Lyn tag or a CAAX motif). As in either the first and the second fusion proteins, the inclusion of a detectable moiety allows for the presence of these proteins in host cells, especially in live cells, to be readily detected and confirmed. These elements in both the first and second fusion proteins may be directly connected between two without any intervening extra amino acids or may be connected by one or more peptide linkers between adjacent elements.

In a second aspect, the present invention provides a nucleic acid comprising a polynucleotide sequence encoding the fusion proteins described above and herein. In some embodiments, the nucleic acid is in the form of an expression cassette, which comprises the polynucleotide sequence encoding the fusion protein being operably linked to a promoter sequence (e.g., a heterologous promoter sequence) directing the expression of the fusion protein. In some embodiments, the nucleic acid is in the form of a vector comprising the polynucleotide sequence or comprising the expression cassette, including a bacterial or viral vector, which may be for the purpose of expressing the fusion protein or merely for the purpose of carrying or transferring the coding sequence. Related to this and the first aspects of the present invention, a composition is further provided, which comprises (1) either the fusion protein or fusion proteins described above and herein or the nucleic acid encoding the fusion protein(s); and (2) at least one physiologically acceptable excipient.

In a third aspect, the present invention provides a genetically modified host cell containing the engineered the fusion protein described above (optionally both the first and second fusion proteins) or the polynucleotide sequence encoding the fusion protein (optionally encoding for both the first and second fusion proteins). In some embodiments, the host cell is a mammalian cell, especially a human cell, for example, a human optic neuronal cell. In some embodiments, the host cell is a live cell located within a person's body, e.g., within an eye. In some embodiments, the first fusion protein contained within the host cell does not comprise a cell membrane-targeting element, but the host cell contains a second fusion protein comprising a membrane-targeting element and a CRY2-binding element, or the host cell contains a polynucleotide sequence encoding for the second fusion protein. In some embodiments, the membrane-targeting element in the second fusion protein is a CAAX motif and the CRY2-binding element is the N-terminal fragment of residues 1-170 of basic helix-loop-helix Arabidopsis protein CIB1 (CIBN). In some embodiments, the host cell is a photoreceptor cell (e.g., a rod cell or a cone cell), a bipolar cell, a retinal ganglion cell (RGC), a corneal epithelial cell, or a Müller cell.

In the fourth aspect, the present invention provides a method for activation of a signal transduction pathway in a cell by delivering an optic signal to the cell. Specifically, the method includes these steps: (i) obtaining a cell comprising the first fusion protein described above; and (ii) exposing the cell to a light within a pre-determined wavelength range for a sufficient amount of time so as to activate the receptor protein's downstream signal transduction pathway. In some embodiments, the activation of the receptor protein's or signaling molecule's downstream signal transduction pathway results in proliferation of the cell. In some embodiments, the activation of the receptor protein's or signaling molecule's downstream signal transduction pathway results in survival and/or regeneration of the cell. In some embodiments, the downstream signaling element of the first fusion protein includes the intracellular domain of tropomyosin receptor kinase B (TrkB, such as rat iTrkB or human iTrkB, shown as the underlined portions in SEQ ID NOs: 2 and 7, respectively), whereas the cell is a human optic neuronal cell, which upon activation of the downstream signal transduction pathway undergoes enhanced cellular proliferation, survival, and/or regeneration. In some embodiments, the human optic neuronal cell is located within a human eye. In some embodiments, the host cell is a photoreceptor cell (e.g., a rod cell or a cone cell), a bipolar cell, a retinal ganglion cell (RGC), a corneal epithelial cell, or a Müller cell. In some embodiments, because the first fusion protein does not comprise a cell membrane-targeting element, a second fusion protein is present in the cell and comprises a cell membrane-targeting element (e.g., a CAAX motif) and a CRY2-binding element (e.g., CIBN, an N-terminal segment of CIB1 protein in a length of less than 200 amino acids). In some embodiments, an N-terminal Lyn tag is present as the cell membrane-targeting element in the first fusion protein and/or the second fusion protein. In some embodiments, the activating light signal is a blue light having a wavelength of about 440 nm to about 490 nm, e.g., 480 nm. In some embodiments, the blue light intensity is 100-1000 μW/cm², or 200-600 μW/cm², or about 400 μW/cm², and the length of time for blue light exposure ranges from 1-6 weeks, or 2-4 weeks, or about 3 weeks. In some embodiments, the method also includes, prior to step (i), a step of introducing into the cell a nucleic acid comprising a polynucleotide sequence encoding the first fusion protein, optionally also a polynucleotide sequence encoding the second fusion protein. In some cases, the first and second fusion proteins are encoded by and therefore introduced into the cell by way of a single viral vector, whereas in other cases, the first and second fusion proteins are encoded by and introduced into the cell by way of two separate viral vectors.

### BRIEF DESCRIPTION OF THE DRAWINGS

Opto-TrkB activates TrkB signaling by blue light and promotes axon regeneration and survival of RGCs after optic nerve injuries. FIG. 1A. Schematic representation of the Opto-TrkB system. Blue light-gated CRY2-CIBN and CRY2-CRY2 interactions recruit the intracellular domain of TrkB to the cell membrane and induce its homo-association, triggering the activation of TrkB signaling. FIG. 1B. Schematic illustrating Opto-TrkB activation in RGCs in mice receiving optic nerve crush (ONC). FIG. 1C. Schematic illustrating the experimental timeline for FIG. 1D-FIG. 1G. ONC was induced 4 weeks after AAV injection. After one day of incubation in the dark, mice either received 2 hours of 600 μW/cm² blue light stimulation or were kept in the dark as a control before tissue analysis. FIG. 1D-FIG. 1E. Sections of retinas from mice expressing Opto-TrkB or GFP were stained with Tuj1 (green) as well as p-ERK (red) (FIG. 1D) or p-AKT (red) (FIG. 1E) antibodies. FIG. 1F-FIG. 1G. Quantification of the percentages of Tuj1+RGCs with p-ERK (FIG. 1D) or p-AKT (FIG. 1E) signals. FIG. 1H. Schematic illustrating the experimental timeline for FIG. 1I-FIG. 1L. After AAV injections, mice were reared in the dark for 4 weeks and received ONC. Then two groups were kept in the dark and two groups under 600 μW/cm² blue light illumination for 2 weeks before tissue analysis. FIG. 1I. Whole-mount retinas from GFP- or Opto-TrkB-expressing mice with surviving RGCs labeled by Tuj1 (green) antibody. FIG. 1J. Quantification of surviving RGCs in I. FIG. 1K. Sections of optic nerves from GFP- or Opto-TrkB expressing mice with RGC axons labeled by CTB-FITC (red). FIG. 1L. Quantification of regenerating axons across different distances in FIG. 1K. ns: not significant, *p≤0.05, **P<0.01, ANOVA followed by Tukey's test, n=3-4 mice (FIG. 1F-FIG. 1G), n=4-5 retinas (FIG. 1J), n=4-5 optic nerves (FIG. 1L). Scale bars, 20 μm (D,E,I), 200 μm (FIG. 1K).

Opto-TrkB induces a multimodal network of signaling events to support axon regeneration. FIG. 2A. Schematic illustrating the experimental timeline and retrograde labeling of RGCs (red) by microruby injection into the optic nerve. FIG. 2B. Schematic illustrating the retina digestion and the collection of microruby labeled RGCs. FIG. 2C. Volcano plots of differentially expressed genes (DEGs) expressed between Opto-TrkB and GFP RGCs harvested on 3 or 14 days after injury. DEGs with adjusted p-values <0.01 and Fold changes >2 were considered significant (Red: Upregulated DEGs in Opto-TrkB groups; Blue: Downregulated DEGs in Opto-TrkB groups). FIG. 2D. Venn diagram showing similarities and differences among all DEGs detected in (C). Boxplots represent the expression of commonly upregulated DEGs in Opto-TrkB 3d or Opto-TrkB 14d groups that are implicated in the “GO:005070 Regulation of axonogenesis” pathway. FIG. 2E. Over-representation analysis (ORA) of DEGs between Opto-TrkB and GFP groups conducted on the Metascape platform. Enrichment of indicated Gene Ontology (GO) functions and corresponding group identities are presented. FIG. 2F. Heatmaps showing the expression profiles of DEGs enriched in “positive regulation of autophagy”, “positive regulation of axonogenesis”, “positive regulation of type I interferon production”, and “IL2 signaling pathway.” DEGs commonly found in 3d and 14d groups are highlighted in red. FIG. 2G. Bubble plots displaying the upregulated Reactome intracellular signaling pathways found in Opto-TrkB 3d and Opto-TrkB 14d groups.

Longer Opto-TrkB activation empowers more axon regeneration and neuroprotection. FIG. 3A. Schematic illustrating the experimental timeline. After ONC induction, mice-expressing Opto-TrkB received blue light exposure at 600 μW/cm² for durations varying from 2 to 14 days. FIG. 3B. Section of optic nerve from GFP-expressing mice at 2 weeks after injury. FIG. 3C. Optic nerve sections from mice expressing Opto-TrkB that received varied durations of blue light stimulations after ONC. FIG. 3D. Quantification of regenerating axons across different distances in B-C. FIG. 3E. Whole-mount retinas from GFP- or Opto-TrkB-expressing mice with surviving RGCs labeled by Tuj1 (green) antibody. FIG. 3F. Quantification of surviving RGCs in E. ns: not significant, *p≤0.05, **P<0.01, ANOVA followed by Tukey's test, n=4-6 optic nerves (FIG. 3D), n=4-6 retinas (FIG. 3F). Scale bars, 200 μm (FIG. 3B, FIG. 3C), 20 μm (FIG. 3E).

Opto-TrkB activation for 4 weeks promotes long-distance axon regeneration. FIG. 4A. Schematic illustrating the experimental timeline. ONC was introduced 4 weeks after the mice received intravitreal AAV2-GFP or AAV2-Opto-TrkB injection in either eye, followed by 4-week blue light stimulation. FIG. 4B. Section of optic nerve from GFP-expressing or Opto-TrkB-expressing eye. White boxes-marked regions indicate distal regions where only long regenerated axons can reach. Scale bar, 500 μm, and 200 μm in zoom-in images. FIG. 4C. Quantification of regenerating axons across different distances in B. **p≤0.01, ANOVA followed by Šídák's test, n=4-5 optic nerves.

Switching on Opto-TrkB signaling prior to axon injury promotes axon regeneration. FIG. 5A. Schematic illustrating the experimental timeline. Mice were kept in the dark throughout the experiments except for one group of Opto-TrkB mice receiving blue light at 600 μW/cm² for 14 days before ONC. FIG. 5B. Section of optic nerve from GFP-expressing mice at 2 weeks after injury. FIG. 5C. Optic nerve sections from mice expressing Opto-TrkB with or without pre-injury blue light stimulations. FIG. 5D. Quantification of regenerating axons across different distances in B-C. FIG. 5E. Whole-mount retinas from GFP- or Opto-TrkB-expressing mice with surviving RGCs labeled by Tuj1 (green). FIG. 5F. Quantification of surviving RGCs in E. ns: not significant among the different groups in F, *p≤0.05, **P<0.01, ANOVA followed by Tukey's test, n=4-6 optic nerves (FIG. 5D), n=4-6 retinas (FIG. 5F). Scale bars, 200 μm (FIG. 5B, FIG. 5C), 20 μm (FIG. 5E).

Adjusting Opto-TrkB activation strength tunes the level of axon regeneration and neuroprotection. FIG. 6A. Schematic illustrating the experimental timeline. After ONC induction, mice expressing Opto-TrkB received blue light exposure at different intensities for 2 weeks. FIG. 6B. Section of optic nerve from GFP-expressing mice at 2 weeks after injury. FIG. 6C. Optic nerve sections from mice expressing Opto-TrkB that received varied intensities of blue light stimulations after ONC. FIG. 6D. Quantification of regenerating axons across different distances in B-C. FIG. 6E. Whole-mount retinas from GFP- or Opto-TrkB-expressing mice with surviving RGCs labeled by Tuj1 (green). FIG. 6F. Quantification of surviving RGCs in E. ns: not significant, *p≤0.05, **P<0.01, ANOVA followed by Tukey's test, n=5-6 optic nerves (FIG. 6D), n=5-6 retinas (FIG. 6F). Scale bars, 200 μm (FIG. 6B, FIG. 6C), 20 μm (FIG. 6E).

Opto-TrkB activation protects RGC survival and brain projection from excitotoxic injury induced by NMDA. FIG. 7A. Time course and schematic of the excitotoxic injury model induced by NMDA to examine the effects of Opto-TrkB activation on RGCs protection and brain projection. FIG. 7B. Whole-mount retinas from GFP- and Opto-TrkB-expressed mice at 2 weeks post PBS or 5 mM NMDA intravitreal injection. The samples were stained with Tuj1 (red) to show the surviving RGCs. FIG. 7C. Quantification of surviving RGCs in B. FIG. 7D. Schematic of RGCs axons projection, which is predominantly to the contralateral side. CTB555 was intravitreally injected into one eye to trace the projection pattern of RGC axons. FIG. 7E. Sections of the optic nerve from GFP- and Opto-TrkB-expressed mice at 2 weeks post PBS or 5 mM NMDA intravitreal injection. The axons were labeled by CTB555. FIG. 7F. Quantification of CTB intensity in E. FIG. 7G. Sections of the SCN from GFP- and OptoTrkB-expressed mice at 2 weeks post PBS or 5 mM NMDA intravitreal injection. FIG. 7H. Quantification of CTB intensity in the SCN. FIG. 7I. Sections of the contralateral LGN from GFP- and Opto-TrkB-expressed mice at 2 weeks post PBS or 5 mM NMDA intravitreal injection. FIG. 7J. Quantification of CTB intensity in the LGN. FIG. 7K. Sections of the contralateral OPN from GFP- and Opto-TrkB-expressed mice at 2 weeks post PBS or 5 mM NMDA intravitreal injection. FIG. 7L. Quantification of CTB intensity in the OPN. FIG. 7M. Sections of the contralateral SC from GFP- and Opto-TrkB-expressed mice at 2 weeks post PBS or 5 mM NMDA intravitreal injection. FIG. 7N. Quantification of CTB intensity in the SC. ns: not significant among the different groups in H, *p≤0.05, **p≤0.01, ANOVA followed by Tukey's test, n=8 retinas (FIG. 7C), n=8 optic nerves (FIG. 7F), n=8 brains (FIG. 7H, FIG. 7J, FIG. 7L, FIG. 7N). Scale bars, 50 μm (FIG. 7B), 200 μm (FIG. 7E, FIG. 7G, FIG. 7I, FIG. 7K, FIG. 7M).

Opto-TrkB activation preserves visual function from NMDA-induced excitotoxic injury. FIG. 8A. Schematic diagram of the optomotor response test. FIG. 8B. Quantification of the visual acuity measured in the optomotor response test. FIG. 8C. Schematic diagram of the looming stimulus test. FIG. 8D. Quantification of mice's responses to the looming visual stimulus. FIG. 8E. Schematic diagram of the visual cliff test. FIG. 8F. Quantification of mice's choices in the visual cliff test without considering any prior behavior of surveying the surroundings. FIG. 8G. Quantification of mice's choices in the visual cliff avoidance test after considering the behavior of surveying the surroundings prior to choosing. FIG. 8H. Quantification of the latency of mice to dismount the center platform in the visual cliff test. FIG. 8I. Images of the eyes showing pupils in the pupillary light reflex test. The pupils constricted to the minimum area were captured and shown in the light ON panel. FIG. 8J. Quantification of the percentage of pupil area changes before and after constriction in the PLR test. FIG. 8K. Quantification of the latency for the maximum pupil constriction. For FIG. 8B, FIG. 8H, FIG. 8J and FIG. 8K, ns: not significant, *p≤0.05, **p≤0.01, ANOVA followed by Tukey's test, n=8-9 mice (FIG. 8B), n=24-26 mice (FIG. 8H), n=14-15 mice (FIG. 8J and FIG. 8K). For FIG. 8D, FIG. 8F and FIG. 8G, p<0.0001 in FIG. 8D, p=0.0578 in FIG. 8F and p=0.0003 in FIG. 8G, Fisher's exact test, n=23-25 mice (FIG. 8D), n=24-26 mice (FIG. 8F and FIG. 8G).

Opo-TrkB can effectively activate downstream signaling and promote neurite growth in PC12 cells. FIG. 9A. Schematic representation of inhibitors suppressing the downstream signaling pathways of light-activated TrkB. Light-induced homo-interaction of iTrkB on the membrane can activate downstream signaling, including ERK and PI3K/Akt pathways, which can be inhibited by U0126 or LY294002, respectively. FIG. 9B. Downstream signaling activities of Opto-TrkB assessed by Western blotting. PC12 cells, transfected with Opot-TrkB or CRY2-mCh+CIBN-GFP-CAAX as a control, were pre-treated with U0126 or/and LY294002 for 1 hr before 1 mW/cm² blue light irradiation for 10 min at 5 sec on/5 sec off intervals. FIG. 9C. Representative images showing the differential levels of PC12 neurite growth under the same set of experimental conditions in (B). Cells were subjected to 0.2 mW/cm² blue light irradiation for 24 hours at 5 sec on/5 sec off intervals. Scale bar, 100 μm. FIG. 9D. Quantification of percentages of PC12 cells with neurite growth under different conditions. >300 cells were counted in each group. Data are presented as mean±s.d. of three independent experiments.

Efficient and specific expression of Opto-TrkB in mice RGCs. FIG. 10 Whole-mount retina from mice injected with AAV-Opto-TrkB (AAV-3HA-CRY2-iTrkB and AAV-CIBN-GFP-CAAX), with HA (red), GFP (green), and Tuj1 (gray, labels RGCs) staining. Over 90% of RGCs were transduced with Opto-TrkB genes. Scale bar, 50 μm.

Opto-TrkB activation matches CNTF's potent efficacy in neuroprotection and axon regeneration, with their combination synergistically boosting long-distance regeneration. Mice were divided into four groups for treatment: AAV-GFP, AAV-Opto-TrkB, AAV-CNTF, and a combination of AAV-Opto-TrkB+AAV-CNTF. Four weeks later, ONC was conducted. Then the Opto-TrkB group and Opto-TrkB+CNTF group were kept in the cage illuminated with 600 μW/cm² blue light for 2 weeks before tissue analysis. FIG. 11A. Sections of retinas stained with Tuj1 (green) as well as p-STAT3 (red) antibodies. FIG. 11B. Quantification of the percentages of Tuj1+RGCs with p-STAT3 signals. FIG. 11C. Sections of optic nerves with RGC axons labeled by CTB-FITC (red). FIG. 11D. Quantification of regenerating axons across different distances in FIG. 11C. FIG. 11E. Whole-mount retinas from each group with surviving RGCs labeled by Tuj1 (green). FIG. 11F. Quantification of surviving RGCs in E. ns: not significant, **P<0.01, ANOVA followed by Tukey's test, n=5 mice (FIG. 11B), n=5 optic nerves (FIG. 11D), n=5 retinas (FIG. 11F). Scale bars, 500 μm (FIG. 11C), 20 μm (FIG. 11A, FIG. 11E).

Four weeks of Opto-TrkB activation promotes RGC survival after optic nerve injury. As shown in the experimental timeline (FIG. 4A), ONC was introduced 4 weeks after the mice received intravitreal AAV2-GFP or AAV2-Opto-TrkB injection in either eye, followed by 4-week blue light stimulation. FIG. 12A. Whole-mount retinas from GFP- or Opto-TrkB-expressing mice with surviving RGCs labeled by Tuj1 (green). Scale bar: 20 μm. FIG. 12B. Quantification of surviving RGCs in A. **p≤0.01, Student's t-test, n=4-5 retinas.

Pre-injury Opto-TrkB activation combined with post-injury activation did not significantly improve RGC survival or axonal regeneration compared to post-injury activation alone. FIG. 13A. Schematic illustrating the experimental timeline. ONC was introduced 4 weeks after intravitreal AAV2-Opto-TrkB injection. Before ONC, mice received pre-injury Opto-TrkB activation for durations ranging from 0 to 28 days, followed by 14 days post-injury Opto-TrkB activation. Blue light exposure was set at 600 μW/cm². FIG. 13B. Optic nerve sections from mice expressing Opto-TrkB that received varied durations of blue light stimulations before ONC. Scale bar, 200 μm. FIG. 13C. Quantification of regenerating axons across different distances in B. FIG. 13D. Whole-mount retinas from Opto-TrkB-expressing mice with surviving RGCs labeled by Tuj1 (green). Scale bar, 20 μm. FIG. 13E. Quantification of surviving RGCs in FIG. 13D. ns: not significant among the different groups, ANOVA followed by Tukey's test, n=4-5 optic nerves (FIG. 13C), n=4-5 retinas (FIG. 13E).

Opto-TrkB activation enhanced αRGC survival post-injury, while inherently resilient melanopsin-positive ipRGCs showed no significant survival rate variation across groups. The experimental design is shown in FIG. 7A. FIG. 14A. Whole-mount retinas from GFP- and Opto-TrkB-expressed mice at 2 weeks post PBS or 5 mM NMDA intravitreal injection. Samples were stained with SMI32 (red) to show the surviving αRGCs. FIG. 14B. Quantification of surviving αRGCs in A. FIG. 14C. Whole-mount retinas from GFP- and Opto-TrkB-expressed mice at 2 weeks post PBS or 5 mM NMDA intravitreal injection. Samples were stained with melanopsin (red) to show surviving ipRGCs. FIG. 14D. Quantification of surviving ipRGCs in C. ns: not significant among the different groups in FIG. 14D, *p≤0.05. ANOVA followed by Tukey's test, n=8 retinas (FIG. 14B, FIG. 14D). Scale bars, 50 μm.

Opto-TrkB activation significantly enhanced neuronal activities in RGC projection-targeted regions after NMDA-induced excitotoxic injury. After performing the behavioral tests in FIG. 8, mice were dark-adapted for 10 hours, followed by 1-hour room light exposure. CTB 555 (green) was intravitreally injected to indicate the SCN, LGN, OPN, and SC regions, while c-Fos staining (red) was used to mark neuronal activities. FIG. 15A, FIG. 15C, FIG. 15E, FIG. 15G. Sections of the SCN (FIG. 15A), contralateral LGN (FIG. 15C), contralateral OPN (FIG. 15E) and contralateral SC (FIG. 15G). FIG. 15B, FIG. 15D, FIG. 15F, FIG. 15H. Quantification of c-fos+ cells in the SCN (FIG. 15B), contralateral LGN (FIG. 15D), contralateral OPN (FIG. 15F) and contralateral SC (FIG. 15H). ns: not significant among the different groups in FIG. 15B, *p≤0.05, **p≤0.01, ANOVA followed by Tukey's test, n=4 brains. Scale bars, 200 μm.

Opto-AKT and Opto-SOScat can promote axon regeneration and survival of RGCs after optic nerve injuries. FIG. 16 Similar to Opto-TrkB system, blue light-gated CRY2-CIBN interactions recruit AKT or SOScat to the cell membrane, triggering the activation of signaling. After AAV injections, mice were reared in the dark for 4 weeks and received ONC, and then were subject to 600 μW/cm² blue light illumination for 2 weeks before tissue analysis. Upper panel: Sections of optic nerves from GFP- or Opto-AKT- or Opto-SOScat-expressing mice with RGC axons labeled by CTB-FITC (red). Quantification of regenerating axons across different distances. Lower panel: Whole-mount retinas from GFP- or Opto-AKT- or Opto-SOScat-expressing mice with surviving RGCs labeled by Tuj1 (green) antibody. Quantification of surviving RGCs. *p≤0.05, **P<0.01, ANOVA followed by Tukey's test, n=4-5 retinas, n=4-5 optic nerves. Scale bars, 20 μm (lower panel), 200 μm (upper panel).

FIG. 17A: light-induced CRY2-CIB1 hetero-binding. FIG. 17B: light-induced CRY2-CRY2 homo-interaction. FIG. 17C: the activation of full-length Trk receptors by neurotrophic factors. FIG. 17D: the design scheme for pre-OptoTrk.

Development and validation of pre-OptoTrkA and pre-OptoTrkB strategies, which are preliminary versions of optogenetic activation of Trk signaling. (FIG. 18A, FIG. 18F) Design scheme of different light-controlled TrkA (FIG. 18A) or TrkB (FIG. 18F) systems, where the homo-interactions of iTrk can be achieved either in the cytosol or on the cell membrane via the CRY2-CIBN or AuLOV toolkits. (FIG. 18B, FIG. 18G) The activation and kinetics of the downstream MAPK pathway activation were assayed by the nuclear translocation of ERK-GFP (FIG. 18A-ii) or the nucleus-to-cytosol translocation of ERK-KTR-GFP (FIG. 18B-ii) in NIH 3T3 cells. The cytosol-to-membrane recruitment of PHAKT1-GFP was used to confirm the activation of the downstream PI3K/AKT signaling (FIG. 18A-ii, B-ii). (FIG. 18C, FIG. 18H) Using the PC12 neurite growth assay, it is confirmed that the pre-OptoTrkA or pre-OptoTrkB system can effectively trigger significant PC12 neurite growth. (FIG. 18D, FIG. 18I) Quantification of percentages of cells with neurite growth for different systems show that the pre-OptoTrkA or pre-OptoTrkB is the most effective strategy in promoting neurite growth in PC12 cells. Results represent mean±SEM values of over 300 cells in each group across three independent sets of experiments. (FIG. 18E, FIG. 18J) Western blot analysis of all the strategies for phosphorylated ERK (Thr202 and Tyr204) and phosphorylated AKT (Ser473) showed that blue light induced the strongest activation of the downstream signaling in pre-OptoTrkA or pre-OptoTrkB systems. Taking together all the results, pre-OptoTrkA or pre-OptoTrkB strategy is the best strategy among all the tested strategies in optically activating Trk signaling. Statistical analysis was performed with one-way ANOVA with Dunnett's post hoc test. Student's t test was used between dark or light cells under the same transfection conditions (*P<0.05, **P<0.005). Scale bars, 10 μm.

pre-OptoTrkA supports DRG neuron survival under the deprivation of NGF. Embryonic DRG neurons were transfected with different optogenetic strategies listed in FIG. 18A-i. After NGF removal from the culture medium, cells were subjected to continuous blue light stimulation at 200 μW/cm² or kept in dark for 2 days. (FIG. 19A-FIG. 19D) Many DRG neurons expressing pre-OptoTrkA were alive with long axons under light exposure, but not in the dark control. However, in cells expressing other strategies, fewer cells survived with light exposure. (FIG. 19E) Quantification of survival rates of DRG neurons shows that pre-OptoTrkA is the most effective in supporting DRG survival in the absence of NGF. t test was performed between cells in dark or in light with the same transfection (*P<0.05, **P<0.005). Scale bars, 50 μm.

FIG. 20: Efficient expression of pre-OptoTrkB system in mice retina. Whole-mount retina from mice injected with both AAV-HA-CRY2-iTrkB and AAV-CIBN-GFP-CAAX, with HA (red), GFP (green), and Tuj1 (gray, labels RGCs) staining. Over 90% of RGCs were transduced with pre-OptoTrkB genes. Scale bar, 50 μm.

Pre-OptoTrkB system activates the downstream Raf/MEK/ERK and PI3K/AKT signaling pathways in RGCs, which can be tuned by light illumination intensities. (FIG. 21A, FIG. 21C) Sections of retinas from mice expressing Pre-OptoTrkB or GFP were stained with Tuj1 (green) as well as p-ERK (red) (FIG. 21A) or p-AKT (red) (FIG. 21C) antibodies. (FIG. 21B, FIG. 21D) Quantification of the percentages of Tuj1+RGCs with p-ERK (FIG. 21B) or p-AKT signals (FIG. 24D). Blue light at 400 μW/cm² yielded higher ratios of p-ERK+ and p-AKT+RGCs than 100 μW/cm² blue light illumination and dark incubation. ns: not significant. **P<0.01, ANOVA followed by Tukey's test, n=4 mice. Scale bars, 50 μm.

Pre-OptoTrkB can promote RGC survival and axonal regeneration in the optic nerve crush injury model of mice. After AAV injections, mice expressing GFP or pre-OptoTrkB were placed in the dark for 4 weeks. (FIG. 22A) Sections of optic nerves from mice stimulated by light or kept in dark for 2 weeks after crush. (FIG. 22B) Numbers of regenerating axons at different distances distal to the lesion site. (FIG. 22C) Survival RGCs in the retina stained with Tuj1. (FIG. 22D) Quantification of Tuj1+RGCs in retinas. There were more axons in the optic nerve and more surviving RGCs in the retina in mice expressing pre-OptoTrkB under light stimulation. ns: not significant. **p≤0.01, ANOVA followed by Tukey's test, n=5-6 mice. Scale bars, 200 μm (A), 50 μm (C).

CRY2(E490R) and CRY2(Δ1-2) obtain enhanced CRY2-CRY2 or CRY2-CIBN binding ability, respectively. (FIG. 23A) Surface charge distribution of CRY2 predicted by homology modeling. Electrostatic charges at N- and C-termini are important for CRY2-CIB1 and CRY2-CRY2 interactions, respectively. (FIG. 23B) Different clustering abilities of CRY2 variants with mutations introduced at positions 489 and 490. Blue light induced different clustering phenomenon of CRY2 variants fused with mCherry in the cytosol of COS-7 cells. (FIG. 23C) Quantification of cluster mass indicates that E490R mutation greatly enhances the clustering ability. FIG. 23D) Integration of CRY2(E490R) in CRY2-Rafl enables stronger optical activation of Raf/MEK/ERK signaling than wild type CRY2 (wtCRY2), as indicated by western blot analysis against p-ERK levels. (FIG. 23E-FIG. 23F) Integration of CRY2(E490R) in pre-OptoTrkA and pre-OptoTrkB can promote stronger PC12 neurite growth than wtCRY2, as indicated by PC12 cell morphologies visualized fluorescence imaging (FIG. 23E) and quantification of percentages of cells with neurite growth (FIG. 23F). (FIG. 23G-FIG. 23H) CRY2 variants with N-terminal truncations (FIG. 23G) or mutations (FIG. 23H) show different light-gated CRY2-CIBN binding abilities, as assessed by yeast 2 hybrid assays where light-mediated CRY2-CIBN association drives the expression of β-galactosidase. Removal of Lys-2 enhanced the CRY2-CIBN binding. Among them, CRY2(Δ1-2) displays the best light-gated CRY2-CIBN binding capability. (FIG. 23I) Integration of CRY2(Δ1-2) in a light-controlled split Cre system yields higher recombination levels than CRY2 wt. Here CRY2 and CIBN binding brings together the split N- and C-fragments of Cre recombinase and thus induces the expression of luciferase reporter in HEK293 cells. Data represent mean values±s.d. (n=3 independent experiments). Scale bars, 10 μm.

FIG. 24A: SOS protein structure FIG. 24B: Diagram of prior Opto-SOS fusion proteins and their light activation. FIG. 24C: Diagram of prior Opto-AKT fusion protein and its light activation.

FIG. 25A: Schematic presentation of OptoSOScat light activation. FIG. 25B: Schematic presentation of OptoAKT light activation.

Optimization and comparison of optogenetic strategies for TrkB activation identify LiTrkB as a potent tool for promoting axon regeneration and neuroprotection. FIG. 26A. Schematic illustrations of full-length TrkB receptor activation and various optogenetic strategies for blue light-inducible TrkB activation. FIG. 26B Membrane-associated oligomerization of the intracellular domain of TrkB (iTrkB) triggered by light-inducible CRY2-CRY2 homo-interactions; FIG. 26C Blue light-gated CRY2-CIBN and CRY2-CRY2 interactions recruit iTrkB to the cell membrane and promotes its homo-association, thereby activating TrkB signaling; FIG. 26D A tandem design using two iLID-SspB modules enables dimerization of iLID-fused iTrkB under blue light irradiation. FIG. 26E. Comparison of downstream p-ERK and p-AKT activation levels by Western blotting. Lentivirus-transduced PC12 cells were stimulated by 1 mW/cm² blue light for 10 min (5 sec on/off intervals). Untreated cells served as a negative control, while full-length TrkB-expressing PC12 cells treated by 25 ng/mL BDNF served as a positive control. A representative blot from three biological replicates is shown. FIG. 26F-FIG. 26G. Quantification of p-ERK (FIG. 26F) and p-Akt (FIG. 26G) levels across the three optogenetic strategies, normalized to the BDNF-treated group. FIG. 26H. Quantification of the percentage of PC12 cells exhibiting neurite outgrowth under different conditions. More than 300 cells per group were analyzed. FIG. 26I. Schematic illustrating LiTrkB activation in RGCs in mice receiving optic nerve crush (ONC) injury. FIG. 26J-FIG. 26K. Sections of retinas from mice expressing LiTrkB were stained with Tuj1 (green) as well as p-ERK (red) (G) or p-AKT (red) (H) antibodies. FIG. 26L-FIG. 26N. Quantification of the percentages of Tuj1+RGCs with p-ERK (FIG. 26L) or p-AKT (FIG. 26M) signals. FIG. 26N. Schematic illustrating the experimental timeline FIG. 26O-FIG. 26R. After AAV injections, mice were reared in the dark for 4 weeks and received ONC. Then two groups were kept in the dark and two groups under 0.6 mW/cm² blue light illumination for 2 weeks before tissue analysis. FIG. 26O. Whole-mount retinas from LiTrkB-expressing mice with surviving RGCs labeled by Tuj1 (green) antibody. FIG. 26P. Quantification of surviving RGCs. FIG. 26Q. Sections of optic nerves from LiTrkB-expressing mice with RGC axons labeled by CTB-FITC (red). FIG. 26R. Quantification of regenerating axons across different distances. ns: not significant, *p≤0.05, **P<0.01. ANOVA followed by Sidak's test (FIG. 26H), ANOVA followed by Tukey's test (FIG. 26L, FIG. 26M, FIG. 26P, FIG. 26R). n=6 technical repeats across three independent experiments (FIG. 26F, FIG. 26G), n=3 independent experiments (FIG. 26H), n=3-4 mice (FIG. 26L-FIG. 26M), n=4-5 retinas (FIG. 26P), n=4-5 optic nerves (FIG. 26R). Scale bars, 20 μm (FIG. 26J, FIG. 26K, FIG. 26O), 200 μm (FIG. 26Q).

Temporal control of LiTrkB activation shows that prolonged activation enhances axon regeneration and neuroprotection, and pre-injury activation promotes axon regeneration. FIG. 27A. Schematic illustrating the experimental timeline for FIG. 27B-FIG. 27D. After ONC induction, mice-expressing LiTrkB received blue light exposure at 0.6 mW/cm² for durations varying from 2 to 14 days. FIG. 27B. Quantification of regenerating axons across different distances in FIG. 27C. FIG. 27C. Optic nerve sections from mice expressing LiTrkB that received varied durations of blue light stimulation after ONC. FIG. 27D. Quantification of surviving RGCs in FIG. 36C. FIG. 27E. Schematic illustrating the experimental timeline for FIG. 27F-FIG. 27H. ONC was introduced 4 weeks after the mice received intravitreal AAV2-GFP or AAV2-LiTrkB injection in either eye, followed by 4-week blue light stimulation. FIG. 27F. Quantification of regenerating axons across different distances in FIG. 27G. FIG. 27G. Section of optic nerve from GFP-expressing or LiTrkB-expressing eye. White boxes-marked regions indicate distal regions where only long regenerated axons can reach. FIG. 27H. Quantification of surviving RGCs in FIG. 36E. FIG. 27I. Schematic illustrating the experimental timeline for FIG. 27J-FIG. 27K. Mice were kept in the dark throughout the experiments except for one group of LiTrkB mice receiving blue light at 0.6 mW/cm² for 14 days before ONC. FIG. 27J. Quantification of regenerating axons across different distances in FIG. 36G. FIG. 27K. Quantification of surviving RGCs in FIG. 36H. ns: not significant, *p≤0.05, **P<0.01. Student's t-test (FIG. 27H), ANOVA followed by Sidak's test (FIG. 27F), ANOVA followed by Tukey's test (FIG. 27B, FIG. 27D, FIG. 27J, FIG. 27K). n=4-6 optic nerves (FIG. 27B), n=4-6 retinas (FIG. 27D), n=4-5 optic nerves (FIG. 27F), n=4-5 retinas (FIG. 27H), n=4-6 optic nerves (FIG. 27J), n=4-6 retinas (FIG. 27K). Scale bars, 200 μm (FIG. 27C, zoom-in images in FIG. 27G), 500 μm (FIG. 27G).

Adjusting LiTrkB activation strength tunes the level of axon regeneration and neuroprotection. FIG. 28A. Schematic illustrating the experimental timeline. After ONC induction, mice expressing LiTrkB received light exposure at different intensities for 2 weeks. FIG. 28B. Section of optic nerve from GFP-expressing mice at 2 weeks after injury. FIG. 28C. Optic nerve sections from mice expressing LiTrkB that received varied intensities of light stimulation after ONC. FIG. 28D. Quantification of regenerating axons across different distances in FIG. 28B-FIG. 28C. FIG. 28E. Whole-mount retinas from GFP- or LiTrkB-expressing mice with surviving RGCs labeled by Tuj1 (green). FIG. 28F. Quantification of surviving RGCs in FIG. 28E. ns: not significant, *p≤0.05, **P<0.01, ANOVA followed by Tukey's test, n=5-6 optic nerves (FIG. 28D), n=5-6 retinas (FIG. 28F). Scale bars, 200 μm (FIG. 28B, FIG. 28C), 20 μm (FIG. 28E).

LiTrkB activation protects RGC survival, brain projection, and preserves visual function from NMDA-induced excitotoxic injury. FIG. 29A. Time course and schematic of the excitotoxic injury model induced by NMDA to examine the effects of LiTrkB activation on RGCs protection and brain projection. FIG. 29B. Whole-mount retinas from GFP- and LiTrkB-expressing mice at 2 weeks post PBS or 5 mM NMDA intravitreal injection. The samples were stained with Tuj1 (red) to show the surviving RGCs. FIG. 29C. Quantification of surviving RGCs in FIG. 29B. FIG. 29D. Schematic of RGCs' axon projection, which is predominantly to the contralateral side. CTB555 was intravitreally injected into one eye to trace the projection pattern of RGC axons. FIG. 29E. Quantification of CTB intensity in the optic nerve (FIG. 29F), SCN (FIG. 38A), contralateral LGN (FIG. 29G), contralateral OPN (FIG. 29H), and contralateral SC (FIG. 29I). FIG. 29F-FIG. 29I. Sections of the optic nerve (FIG. 29F), contralateral LGN (FIG. 29G), contralateral OPN (FIG. 29H), and contralateral SC (FIG. 29I) from GFP- and LiTrkB-expressing mice at 2 weeks post PBS or 5 mM NMDA intravitreal injection. The axons were labeled by CTB555. FIG. 29J. Schematic diagram of the optomotor response test. FIG. 29K. Quantification of the visual acuity measured in the optomotor response test. FIG. 29L. Schematic diagram of the looming stimulus test. FIG. 29M. Quantification of mice's responses to the looming visual stimulus. FIG. 29N. Schematic diagram of the visual cliff test. FIG. 29O. Quantification of mice's choices in the visual cliff test without considering any prior behavior of surveying the surroundings. FIG. 29P. Quantification of mice's choices in the visual cliff avoidance test after considering the behavior of surveying the surroundings prior to choosing. FIG. 29Q. Quantification of the latency of mice to dismount the center platform in the visual cliff test. ns: not significant, *p≤0.05, **p≤0.01. ANOVA followed by Tukey's test (FIG. 29C, FIG. 29E, FIG. 29K, FIG. 29Q), Fisher's exact test (FIG. 29M, FIG. 29O, FIG. 29P). n=8 retinas (FIG. 29C), n=8 optic nerves or 8 brains (FIG. 29E), n=8-9 mice (FIG. 29K), n=23-25 mice (FIG. 29M), n=24-26 mice (FIG. 29O-FIG. 29Q). Scale bars, 100 μm (FIG. 29B), 200 μm (FIG. 29F-FIG. 29I).

Post-injury LiTrkB activation in the excitotoxic injury model restores visual functions. FIG. 30A. Time course and schematic of post-injury LiTrkB activation in the NMDA-induced excitotoxic injury model. FIG. 30B. Number of mice responding to the looming stimulus before NMDA treatment and at various time points following light stimulation. FIG. 30C. Quantification of the mice's responses to the looming visual stimulus 12 weeks after light stimulation. FIG. 30D. Quantification of visual acuity 12 weeks after light stimulation. FIG. 30E. Quantification of surviving RGCs in FIG. 40B. FIG. 30F. Quantification of CTB intensity in the optic nerve (FIG. 40C), contralateral LGN (FIG. 30G), contralateral SC (FIG. 30H), contralateral OPN, and SCN. FIG. 30G-FIG. 30H. Sections of the contralateral LGN (FIG. 30G) and contralateral SC (FIG. 30H) from GFP- or LiTrkB-expressing mice treated with PBS or 5 mM NMDA, 12 weeks following light stimulation. FIG. 30I. Sholl analysis of RGC dendritic morphology in intact or NMDA-injured retinas after 12 weeks of control or LiTrkB activation treatment. FIG. 30J. Representative skeleton images show the dendritic arbors of Thy1-GFP labeled αRGCs from intact or NMDA-injured retinas after 12 weeks of control or LiTrkB activation treatment. The skeleton images were traced with the ImageJ plugin Simple Neurite Tracer, based on the images in FIG. 40D. ns: not significant, *p≤0.05, **p≤0.01. ANOVA followed by Tukey's test (FIG. 30D, FIG. 30E, FIG. 30F), Fisher's exact test (FIG. 30C), and ANOVA followed by Bonferroni's test (FIG. 30I). n=12 mice (FIG. 30C, FIG. 30D), n=10 retinas or 10 brains (FIG. 30E, FIG. 30F), n=11-29 RGCs. Scale bars, 200 μm (FIG. 30G, FIG. 30H), 100 μm (FIG. 30J).

LiTrkB activation mitigates RGC degeneration in a glaucoma model. FIG. 31A. Time course and diagram of the experiment examining the effects of LiTrkB activation in the glaucoma model induced by SO intracameral injection. FIG. 31B. Quantification of IOP before and at various time points after SO injection. FIG. 31C. Schematic diagram of the retinal flat mount process and different retinal regions. FIG. 31D. Quantification of RGC survival rate in the peripheral, middle, and central retina at 8 weeks after SO injection. FIG. 31E. Whole-mount retinas from mice expressing GFP or LiTrkB, stained with RBPMS (red) to show surviving RGCs, at 8 weeks after SO injection. Additional retinas are shown in FIG. 42. FIG. 31F. Enlarged images of the peripheral, middle, and central retina from GFP- and LiTrkB-expressing mice at 8 weeks after SO injection. FIG. 31G. Quantification of visual acuity 8 weeks after SO injection. *p≤0.05, **p≤0.01. ANOVA followed by Bonferroni's test. n=10 eyes (FIG. 31B, FIG. 31G, FIG. 31D). Scale bars, 1 mm (FIG. 31E), 100 μm (FIG. 31F).

Late-stage LiTrkB activation restores visual functions in the glaucoma model. FIG. 32A. Time course of late-stage LiTrkB activation in the glaucoma model. FIG. 32B. Quantification of visual acuity from 4 to 16 weeks after SO injection. FIG. 32C. Quantification of visual acuity at 4 weeks, 14 weeks, and 16 weeks post-SO injection. FIG. 32D. Quantification of RGC survival rate in the peripheral, middle, and central retina at 16 weeks after SO injection. FIG. 32E, FIG. 32G. Quantification of CTB intensity in the contralateral LGN (FIG. 32E) and contralateral SC (FIG. 32G). FIG. 32F, FIG. 32H. Sections of the contralateral LGN (FIG. 32F) and contralateral SC (FIG. 32H) from mice expressing GFP or LiTrkB, treated with PBS or SO, 16 weeks post-injection. FIG. 32I. Sholl analysis of RGC dendritic morphology from retinas of intact mice or those 16 weeks post-SO injection. FIG. 32J. Representative skeleton images displaying the dendritic arbors of PRV-RFP-labeled αRGCs from retinas of intact mice or those 16 weeks post-SO injection. ns: not significant, *p≤0.05, **p≤0.01. ANOVA followed by Bonferroni's test (FIG. 32B-FIG. 32G, FIG. 32I). n=18 (FIG. 32B, FIG. 32C), n=12 (FIG. 32D-FIG. 32G), n=31-43 RGCs. Scale bars, 200 μm (FIG. 32F, FIG. 32H), 100 μm (FIG. 32J).

LiTrkB can effectively activate downstream signaling and promote neurite growth in PC12 cells. FIG. 33A. Lentivirus-transduced PC12 cells expressing different optogenetic constructs had comparable mRNA levels of the intracellular TrkB domain. RT-qPCR data are presented as mean±SEM of four repeats from two independent samples. FIG. 33B. Representative images showing differences in neurite growth in PC12 cells. Cells were exposed to 0.2 mW/cm² blue light for 24 h (5 sec on/off intervals). Scale bar: 50 μm. FIG. 33C. Schematic representation of inhibitors suppressing the downstream signaling pathways of light-activated TrkB. Light-induced homo-interaction of iTrkB on the membrane can activate downstream signaling, including ERK and PI3K/Akt pathways, which can be inhibited by U0126 or LY294002, respectively. FIG. 33D. Downstream signaling activities of LiTrkB assessed by Western blotting. PC12 cells, transfected with Opoto-TrkB or CRY2-mCh+CIBN-GFP-CAAX as a control, were pre-treated with U0126 or/and LY294002 for 1 hr before 1 mW/cm² blue light irradiation for 10 min at 5 sec on/5 sec off intervals. FIG. 33E. Representative images showing the differential levels of PC12 neurite growth under the same set of experimental conditions in (FIG. 33B). Cells were subjected to 0.2 mW/cm² blue light irradiation for 24 hours at 5 sec on/5 sec off intervals. Scale bar, 100 μm. FIG. 33F. Quantification of percentages of PC12 cells with neurite growth under different conditions. >300 cells were counted in each group. Data are presented as mean±s.d. of three independent experiments.

Efficient expression of LiTrkB in RGCs. FIG. 34A. Whole-mount retina from mice injected with AAV-LiTrkB (AAV-3HA-CRY2-iTrkB and AAV-CIBN-GFP-CAAX), with HA (red), GFP (green), and Tuj1 (gray, labels RGCs) staining. Over 90% of RGCs were transduced with LiTrkB genes. FIG. 34B. Schematic illustrating the experimental timeline for FIG. 1G-FIG. 1J. ONC was induced 4 weeks after AAV injection. After one day of incubation in the dark, mice either received 2 hours of 0.6 mW/cm² blue light stimulation or were kept in the dark as a control before tissue analysis. FIG. 34C, FIG. 34D. Sections of retinas from mice expressing GFP were stained with Tuj1 (green) as well as p-ERK (red) (FIG. 34C) or p-AKT (red) (FIG. 34D) antibodies. FIG. 34E. Whole-mount retinas from GFP-expressing mice with surviving RGCs labeled by Tuj1 (green) antibody. F. Sections of optic nerves from GFP-expressing mice with RGC axons labeled by CTB-FITC (red). Scale bars, 50 μm (FIG. 34A), 20 μm (FIG. 34C-FIG. 34E), 200 μm (FIG. 34F).

LiTrkB activation matches CNTF's potent efficacy in neuroprotection and axon regeneration, with their combination synergistically boosting long-distance regeneration. Mice were divided into four groups for treatment: AAV-GFP, AAV-LiTrkB, AAV-CNTF, and a combination of AAV-LiTrkB+AAV-CNTF. Four weeks later, ONC was conducted. Then the LiTrkB group and LiTrkB+CNTF group were kept in the cage illuminated with 0.6 mW/cm² blue light for 2 weeks before tissue analysis. FIG. 35A. Sections of retinas stained with Tuj1 (green) as well as p-STAT3 (red) antibodies. AAV-mediated expression of CNTF effectively activates the downstream STAT3 signaling pathway. FIG. 35B. Quantification of the percentages of Tuj1+RGCs with p-STAT3 signals. FIG. 35C. Sections of optic nerves with RGC axons labeled by CTB-FITC (red). FIG. 35D. Quantification of regenerating axons across different distances in FIG. 35C. FIG. 35E. Whole-mount retinas from each group with surviving RGCs labeled by Tuj1 (green). FIG. 35F. Quantification of surviving RGCs in FIG. 35E. ns: not significant, **P<0.01, ANOVA followed by Tukey's test, n=5 mice (FIG. 35B), n=5 optic nerves (FIG. 35D), n=5 retinas (FIG. 35F). Scale bars, 500 μm (FIG. 35C), 20 μm (FIG. 35A, FIG. 35E).

Temporal control of LiTrkB activation shows that prolonged activation enhances axon regeneration and neuroprotection, and pre-injury activation promotes axon regeneration. FIG. 36A. Schematic illustrating the experimental timeline for B-C. FIG. 36B. Section of optic nerve from GFP-expressing mice with 14 days of light exposure at 2 weeks after injury. FIG. 36C. Whole-mount retinas from GFP- or LiTrkB-expressing mice with surviving RGCs labeled by Tuj1 (green) antibody. FIG. 36D. Schematic illustrating the experimental timeline for FIG. 36E. FIG. 36E. Whole-mount retinas from GFP- or LiTrkB-expressing mice 4 weeks after ONC with surviving RGCs labeled by Tuj1 (green). FIG. 36F. Schematic illustrating the experimental timeline for FIG. 36G-FIG. 36H. FIG. 36G. Section of optic nerve from GFP-expressing mice and LiTrkB-expressing mice with or without pre-injury blue light stimulations at 2 weeks after injury. FIG. 36H. Whole-mount retinas from GFP- or LiTrkB-expressing mice with surviving RGCs labeled by Tuj1 (green). Scale bars, 200 μm (FIG. 36B, FIG. 36G), 20 μm (FIG. 36C, FIG. 36E, FIG. 36H).

Intravitreous injection of NMDA specifically caused RGC death, and LiTrkB activation enhanced αRGC survival post-injury, while inherently resilient melanopsin-positive ipRGCs showed no significant survival rate variation across groups. The experimental design is shown in FIG. 29A. FIG. 37A. Sections of retinas from mice with PBS or 5 mM NMDA intravitreous injection for 14 days, stained with Tuj1 (red) and DAPI (blue). IPL: Inner Plexiform Layer; INL: Inner Nuclear Layer; OPL: Outer Plexiform Layer; ONL: Outer Nuclear Layer. FIG. 37B. Quantification of the thickness of various retinal layers. FIG. 37C. Whole-mount retinas from GFP- and LiTrkB-expressing mice at 2 weeks post PBS or 5 mM NMDA intravitreal injection. Samples were stained with SMI32 (red) to show the surviving αRGCs. FIG. 37D. Quantification of surviving αRGCs in FIG. 37C. FIG. 37E. Whole-mount retinas from GFP- and LiTrkB-expressed mice at 2 weeks post PBS or 5 mM NMDA intravitreal injection. Samples were stained with melanopsin (red) to show the surviving ipRGCs. FIG. 37F. Quantification of surviving ipRGCs in FIG. 37E. ns: not significant, *p≤0.05, **P<0.01. Student's t-test (FIG. 37B), and ANOVA followed by Tukey's test (FIG. 37D, FIG. 37F). n=5 retinas (FIG. 37B), n=8 retinas (FIG. 37D, FIG. 37F). Scale bars, 50 μm (FIG. 37A, FIG. 37C, FIG. 37E).

LiTrkB activation preserves visual function from NMDA-induced excitotoxic injury. FIG. 38A. Sections of the SCN from GFP- and OptoTrkB-expressing mice at 2 weeks post PBS or 5 mM NMDA intravitreal injection. FIG. 38B. Images of the eyes showing pupils in the pupillary light reflex test. The pupils constricted to the minimum area were captured and shown in the light ON panel. FIG. 38C. Quantification of the percentage of pupil area changes before and after constriction in the PLR test. FIG. 38D. Quantification of the latency for the maximum pupil constriction. ns: not significant, *p≤0.05, **p≤0.01. ANOVA followed by Tukey's test. n=14-15 mice (FIG. 38C, FIG. 38D). Scale bar, 200 μm (FIG. 38A).

LiTrkB activation significantly enhanced neuronal activities in RGC projection-targeted regions after NMDA-induced excitotoxic injury. After performing the behavioral tests in FIG. 29, mice were dark-adapted for 10 hours, followed by 1-hour room light exposure. CTB 555 (green) was intravitreally injected to indicate the SCN, LGN, OPN, and SC regions, while c-Fos staining (red) was used to mark neuronal activities. FIG. 39A, FIG. 39C, FIG. 39E, FIG. 39G. Sections of the SCN (FIG. 39A), contralateral LGN (FIG. 39C), contralateral OPN (FIG. 39E), and contralateral SC (FIG. 39G). FIG. 39B, FIG. 39D, FIG. 39F, FIG. 39H. Quantification of c-fos+ cells in the SCN (FIG. 39B), contralateral LGN (FIG. 39D), contralateral OPN (FIG. 39F), and contralateral SC (FIG. 39H). ns: not significant, *p ≤0.05, **p≤0.01, ANOVA followed by Tukey's test, n=4 brains. Scale bars, 200 μm.

Post-injury LiTrkB activation does not enhance RGC survival but promotes RGC dendritic growth. FIG. 40A. Quantification of surviving RGCs following a 7-day intravitreal injection of PBS or at various time points after an intravitreal injection of 5 mM NMDA. FIG. 40B. Whole-mount retinas from mice expressing GFP and LiTrkB were collected 12 weeks after light stimulation and treated with either PBS or 5 mM NMDA. FIG. 40C. Sections of the optic nerve from GFP- and LiTrkB-expressed mice were collected 12 weeks after light stimulation and treated with either PBS or 5 mM NMDA. FIG. 40D. Representative images of dendritic arbors from Thy1-GFP-labeled RGCs in GFP- and LiTrkB-expressed mice were obtained 12 weeks after light stimulation. The samples were treated with either PBS or 5 mM NMDA, and SMI32 staining was used to demonstrate the co-localization of αRGCs with GFP in the Thy 1-GFP mouse line. ns: not significant, **p≤0.01, ANOVA followed by Tukey's test, n=3 (FIG. 40A). Scale bars, 100 μm (FIG. 40B, FIG. 40D), 200 μm (FIG. 40C).

LiTrkB activation mitigates RGC degeneration four weeks after glaucoma induction. FIG. 41A. Quantification of IOP before and at various time points after SO injection. FIG. 41B. Quantification of RGC survival rate in the peripheral, middle, and central retina 4 weeks after SO injection. FIG. 41C. Whole-mount retinas from mice expressing GFP or LiTrkB, stained with RBPMS (red) to show surviving RGCs, at 4 weeks after SO injection. FIG. 41D. Diagram of different retinal regions and enlarged images of the peripheral, middle, and central retina from GFP- and LiTrkB-expressed mice 4 weeks after SO injection. *p≤0.05, **p≤0.01, ANOVA followed by Bonferroni's test, n=8 retinas. Scale bars, 1 mm (FIG. 41C), 100 μm (FIG. 41D).

LiTrkB activation reduces RGC degeneration eight weeks after glaucoma induction. (FIG. 42A-FIG. 42C) Whole-mount retinas from GFP- or LiTrkB-expressing mice treated with PBS or SO for 8 weeks, immunostained with RBPMS (red) to label surviving RGCs. Scale bars, 1 mm.

Late-stage LiTrkB activation restores visual functions in the glaucoma model. FIG. 43A. Quantification of RGC survival rates in the peripheral, middle, and central retina 4 weeks after SO injection, along with whole-mount retinas from mice expressing GFP or LiTrkB, stained with RBPMS (red) to highlight surviving RGCs at 4 weeks post-SO injection. FIG. 43B. Whole-mount retinas from mice expressing GFP or LiTrkB 16 weeks after PBS or SO injection. FIG. 43C. Sections of the SC from mice following 36 hours of PRV-RFP injection. FIG. 43D. Representative images of dendritic arbors from PRV-RFP-labeled RGCs, with SMI32 staining to identify αRGCs. Scale bars, 1 mm (FIG. 43A, FIG. 43B), 200 μm (FIG. 43C), 100 μm (FIG. 43D).

### DEFINITIONS

The term “nucleic acid” or “polynucleotide” refers to deoxyribonucleotides or ribonucleotides and polymers thereof in either single- or double-stranded form. Unless specifically limited, the term encompasses nucleic acids containing known analogues of natural nucleotides which have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions) and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res., 19:5081 (1991); Ohtsuka et al., J. Biol. Chem., 260:2605-2608 (1985); and Cassol et al., (1992); Rossolini et al., Mol. Cell. Probes, 8:91-98 (1994)). The terms nucleic acid and polynucleotide are used interchangeably with gene, cDNA, and mRNA encoded by a gene.

The terms “polypeptide,” “peptide,” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid (e.g., D-amino acid) polymers. As used herein, the terms encompass amino acid chains of any length, including full-length proteins (i.e., antigens), wherein the amino acid residues are linked by covalent peptide bonds.

The term “amino acid” refers to naturally occurring and synthetic amino acids, as well as amino acid analogs and amino acid mimetics that function in a manner similar to the naturally occurring amino acids, including D-amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. “Amino acid mimetics” refers to chemical compounds that have a structure that is different from the general chemical structure of an amino acid, but that functions in a manner similar to a naturally occurring amino acid.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

An “expression cassette” is a nucleic acid construct, generated recombinantly or synthetically, with a series of specified nucleic acid elements that permit transcription of a particular polynucleotide sequence in a host cell. An expression cassette may be part of a plasmid, viral genome, or nucleic acid fragment. It may be a DNA construct or in an RNA version. Typically, an expression cassette includes a polynucleotide sequence to be transcribed, operably linked to a promoter. “Operably linked” in this context means two or more genetic elements, such as a polynucleotide coding sequence and a promoter, placed in relative positions that permit the proper biological functioning of the elements, such as the promoter directing transcription of the coding sequence. Other elements that may be present in an expression cassette include those that enhance transcription (e.g., enhancers) and terminate transcription (e.g., terminators), as well as those that confer certain binding affinity or antigenicity to the recombinant protein produced from the expression cassette.

The term “heterologous,” as used in the context of describing the relative location of two elements, refers to the two elements such as two polynucleotide sequences (e.g., a promoter and a polypeptide-encoding sequence) or polypeptide sequences (e.g., a first amino acid sequence and a second peptide sequence serving as a fusion partner with the first amino acid sequence in a fusion protein) that are not naturally found in the same relative position. Thus, a “heterologous promoter” of a gene refers to a promoter that is not naturally operably linked to that gene. Similarly, a “heterologous polypeptide/amino acid sequence” or “heterologous polynucleotide” to an amino acid sequence or its encoding sequence is one derived from an origin different from the amino acid sequence or its encoding sequence.

As used in this application, an “increase” or a “decrease” refers to a detectable positive or negative change in quantity (such as the level of downstream signaling event) from a comparison basis, e.g., an established standard control level. An increase is a positive change that is typically at least 10%, or at least 20%, or 50%, or 100%, and can be as high as at least 2-fold or at least 5-fold or even 10-fold of the control value. Similarly, a decrease is a negative change that is typically at least 10%, or at least 20%, 30%, or 50%, or even as high as at least 80% or 90% of the control value. Other terms indicating quantitative changes or differences from a comparative basis, such as “more,” “less,” “higher,” and “lower,” as well as terms indicating an action to cause such changes or differences, such as “increase,” “promote,” “enhance,” “decrease,” “inhibit,” and “suppress,” are used in this application in the same fashion as described above. In contrast, the term “substantially the same” or “substantially lack of change” indicates little to no change in quantity from the standard control value, typically within ±10% of the standard control, or within ±5%, 2%, or even less variation from the standard control.

As used herein, an “effective amount” or a “therapeutically effective amount” means the amount of a compound that, when administered to a subject or patient for treating a disorder, is sufficient to prevent, reduce the frequency of, or alleviate the symptoms of the disorder. The effective amount will vary depending on a variety of the factors, such as a particular compound used, the disease and its severity, the age, weight, and other factors of the subject to be treated. Amelioration of a symptom of a particular condition by administration of a pharmaceutical composition described herein refers to any lessening, whether permanent or temporary, that can be associated with the administration of the pharmaceutical composition. For example, the amount of a fusion protein of this invention or its corresponding encoding nucleic acid is considered therapeutically effective for treating a condition involving impaired or lost biological functions or responses (e.g., lost or impaired sensory perception such as vision) when treatment results in eliminated symptoms, delayed onset of symptoms, or reduced frequency or severity of symptoms, including partially or fully restored biological functionality, etc.

As used herein, the term “treatment” or “treating” includes both therapeutic and preventative measures taken to address the presence of a disease or condition or the risk of developing such disease or condition at a later time. It encompasses therapeutic or preventive measures for alleviating ongoing symptoms, inhibiting or reversing disease progression, delaying of onset of symptoms, or eliminating or reducing or reversing sequelae caused by such disease or condition. A preventive measure in this context and its variations do not require 100% elimination of the occurrence of an event; rather, they refer to a suppression or reduction in the likelihood or severity of such occurrence or a delay in such occurrence.

A “subject,” or “subject in need of treatment,” as used herein, refers to an individual who seeks medical attention due to risk of, or actual sufference from, a condition involving the loss or impaired biological/physiological perception or response. The term subject can include both animals, especially mammals, and humans. Subjects or individuals in need of treatment include those that demonstrate symptoms relating to diseases or conditions caused or exacerbated by the loss or impairment of a biological/physiological function or are at risk of later developing these conditions and/or related symptoms.

As used herein, the term “about” denotes a range of +/−10% of a predetermined value. For instance, “about 10” denotes a range of 10±1, i.e., a range of 9-11.

### DETAILED DESCRIPTION OF THE INVENTION

### I. Introduction

Various signaling pathways are known to play critical roles in the regulation of cellular processes such as cell proliferation, differentiation, survival, migration, regeneration, etc. As these processes are directly relevant to many physiological states and pathological conditions, there exists a keen interest to effectively regulate and control pertinent cellular signal transduction pathways. One strategy to exert easy and efficient control of signal transduction is through the use of a genetically engineered upstream signaling molecule that directly responds to a light signal rather than the binding of an extracellular ligand.

Neuroprotective treatments for retinal ganglion cells (RGCs) hold great promise to halt the progression or even cure many eye diseases. Tremendous efforts during the last several decades have identified nerve growth factor (NGF) and brain-derived neurotrophic factor (BDNF) as potent neuroprotective factors for RGCs. NGF and BDNF belong to neurotrophins, an essential family of secreted proteins for neuronal development and survival. NGF and BNDF bind with high affinity to the tropomyosin receptor kinases TrkA and TrkB, respectively. Upon RGC damage, the expression and activation of TrkA and TrkB in RGCs are altered while NGF and BDNF levels are reduced. Preclinical studies have demonstrated that enhancement of NGF/TrkA or BDNF/TrkB signaling can prevent or slow down RGC death in different animal models of eye diseases. Various approaches from different aspects have been explored in order to augment Trk signaling pathways in RGCs that are located near the inner surface of the retina (see, e.g., Mallone et al., Appl Sci-Basel 2020, 10 (24), p. 9015).

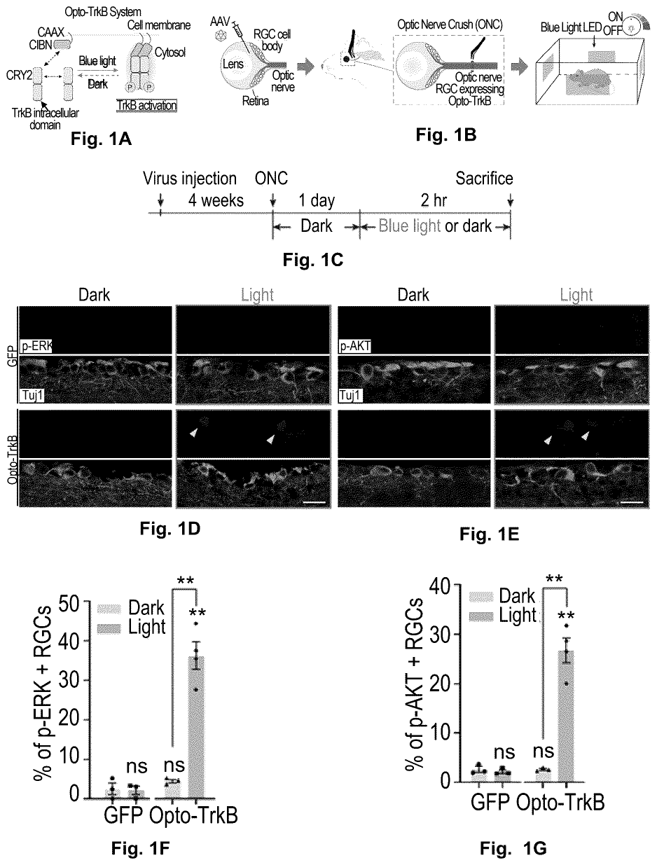

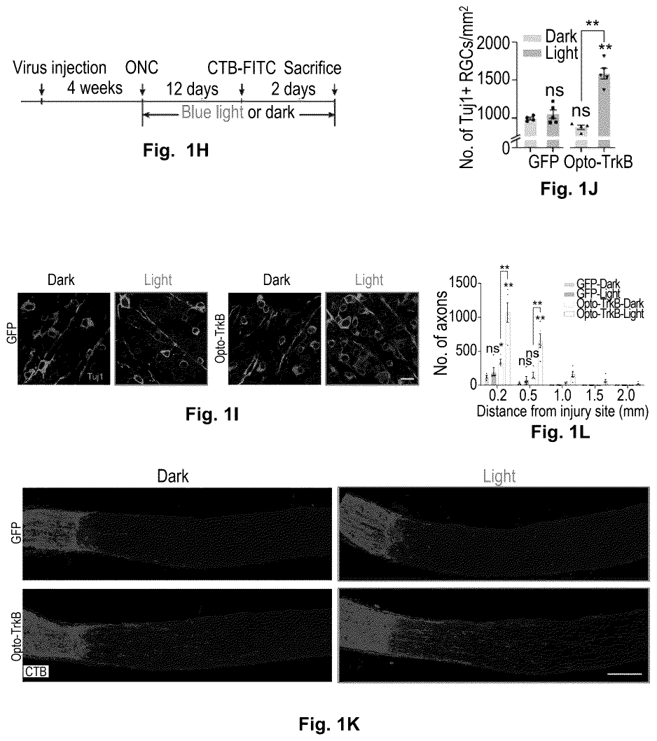

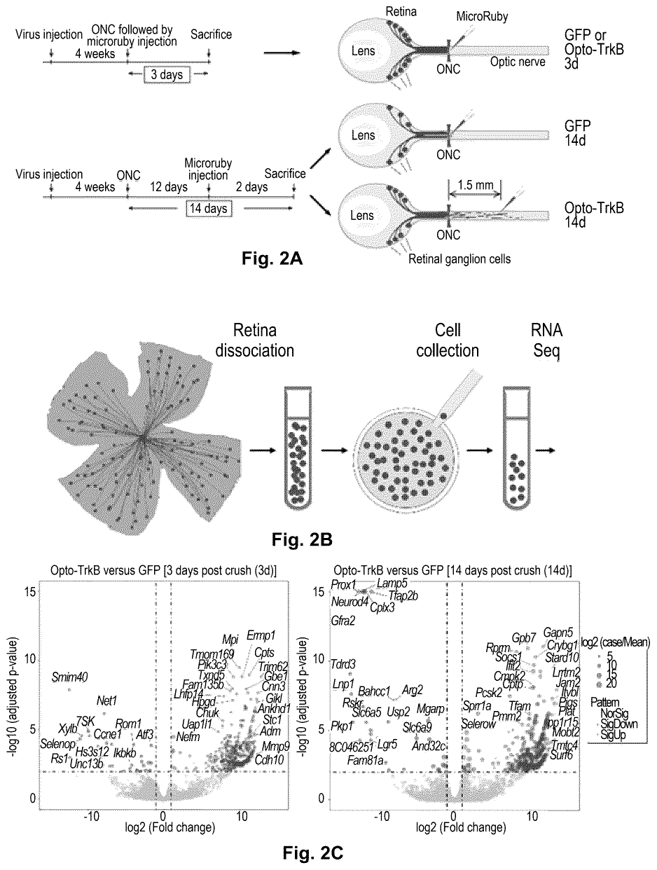

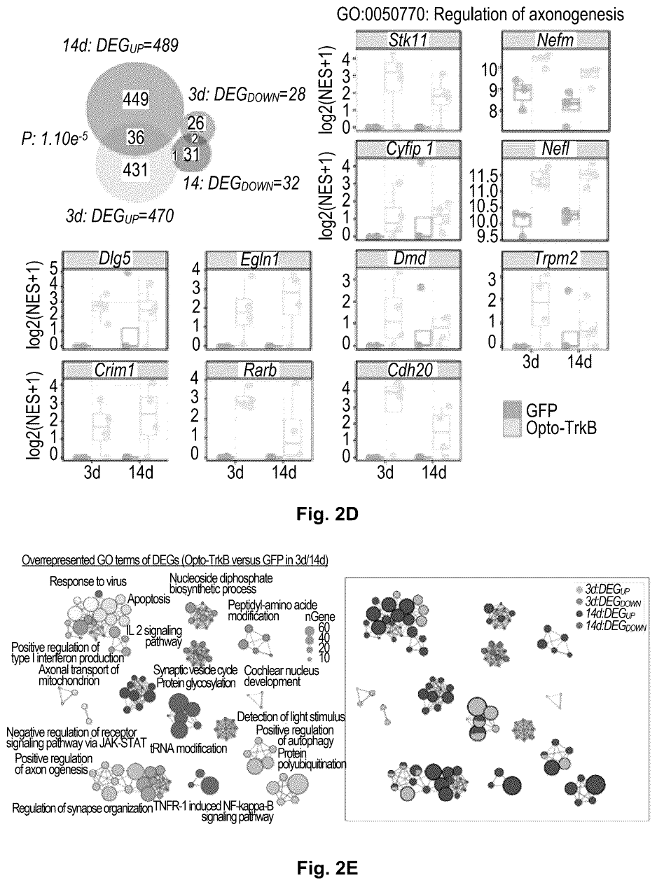

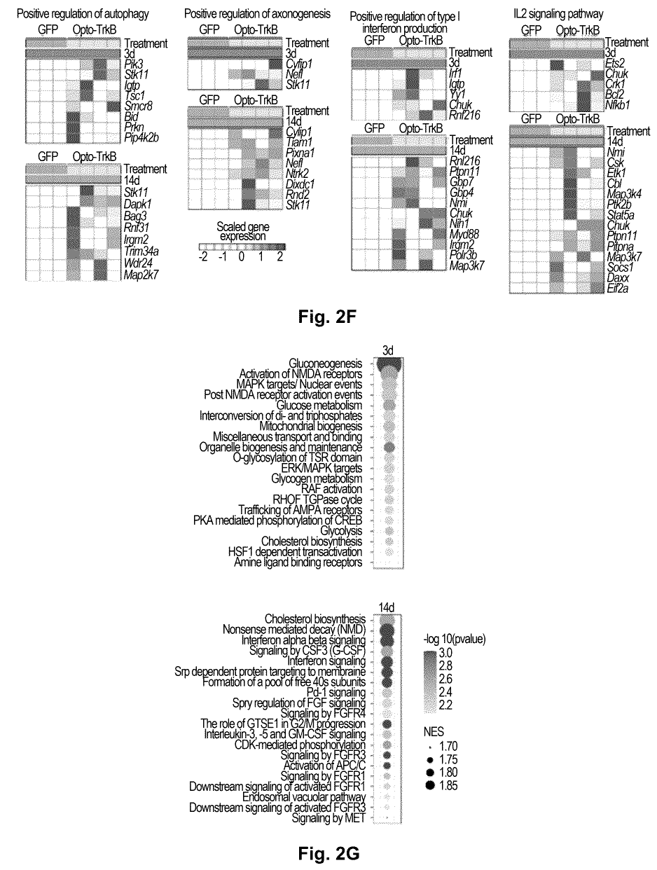

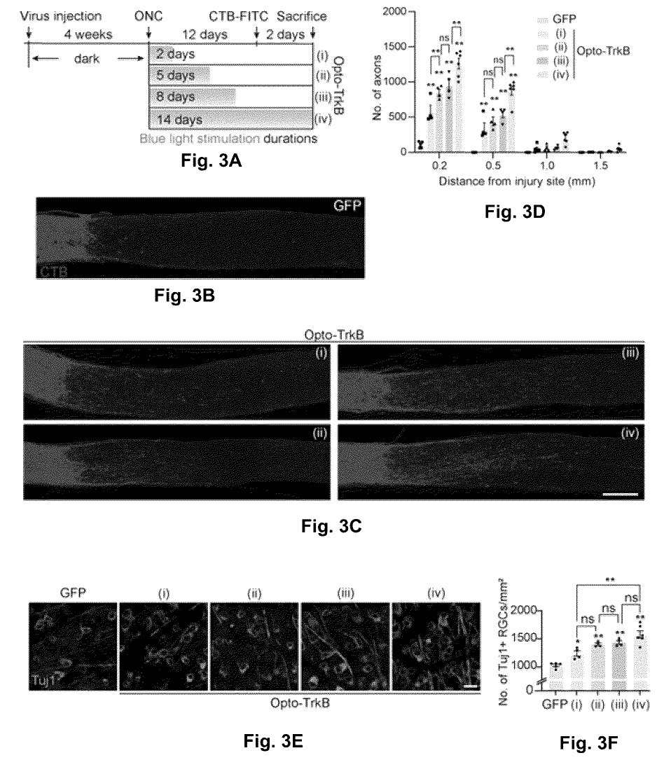

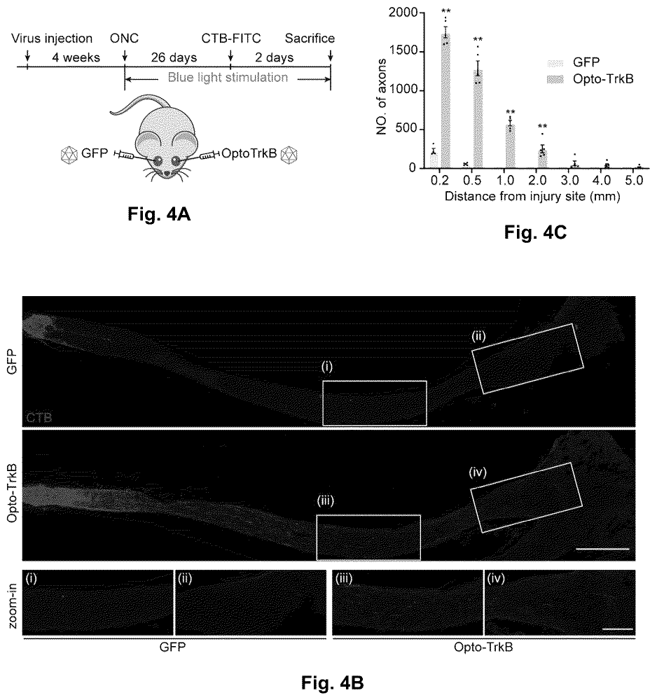

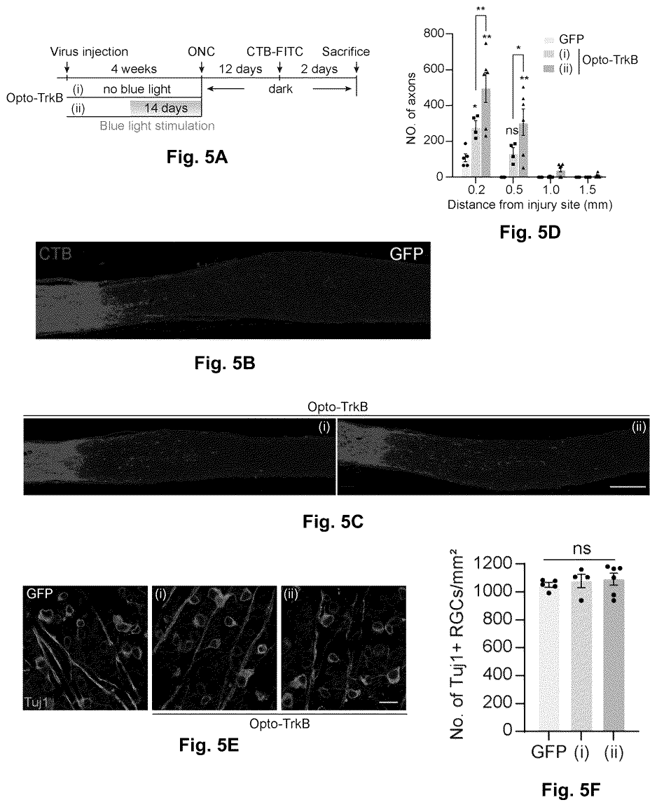

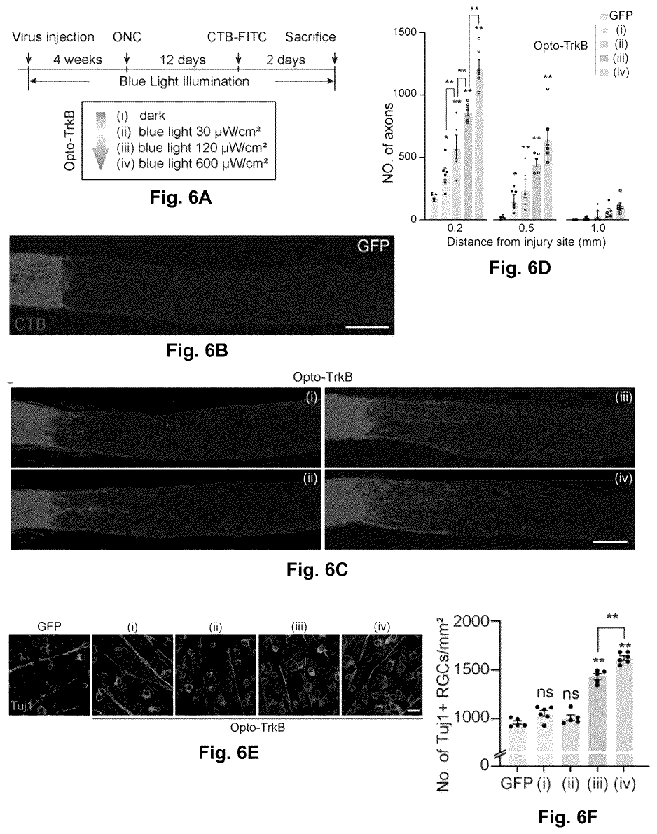

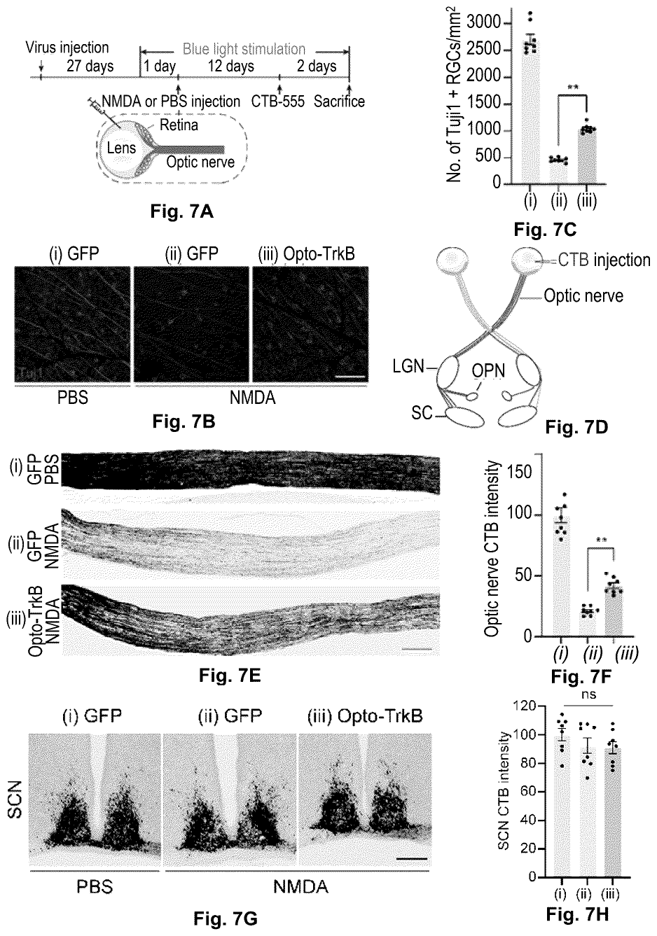

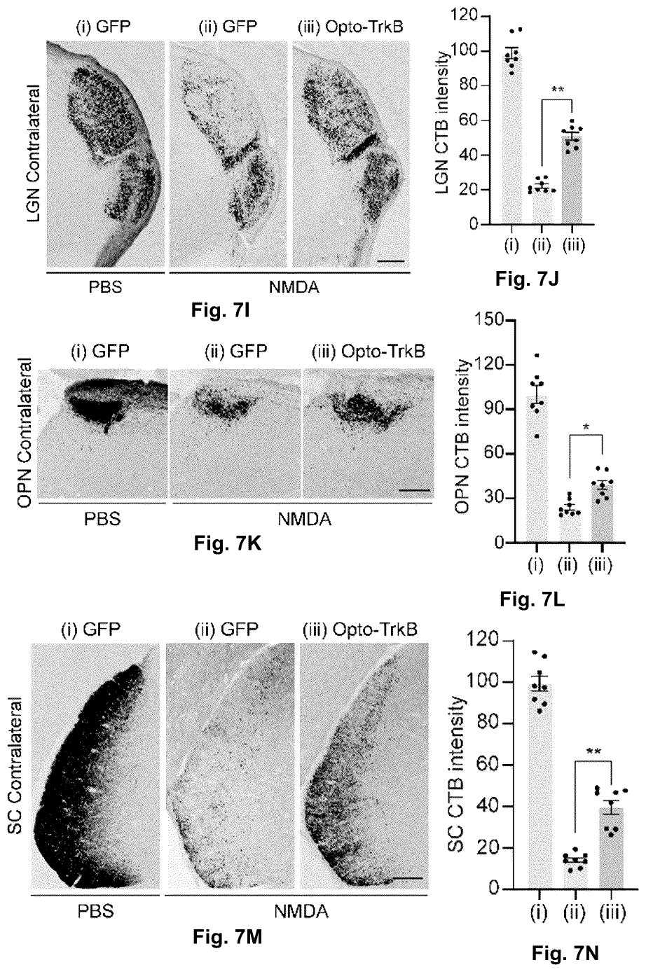

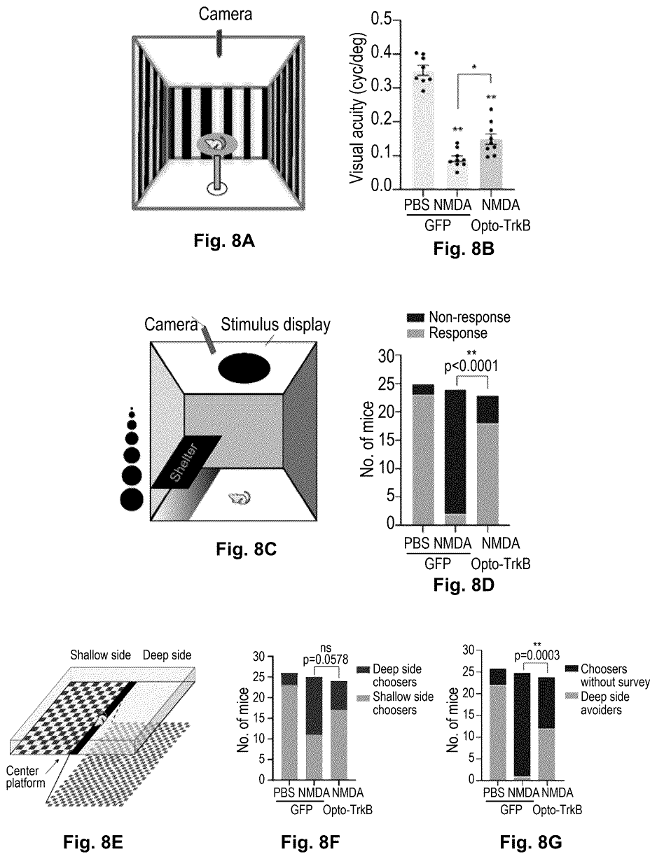

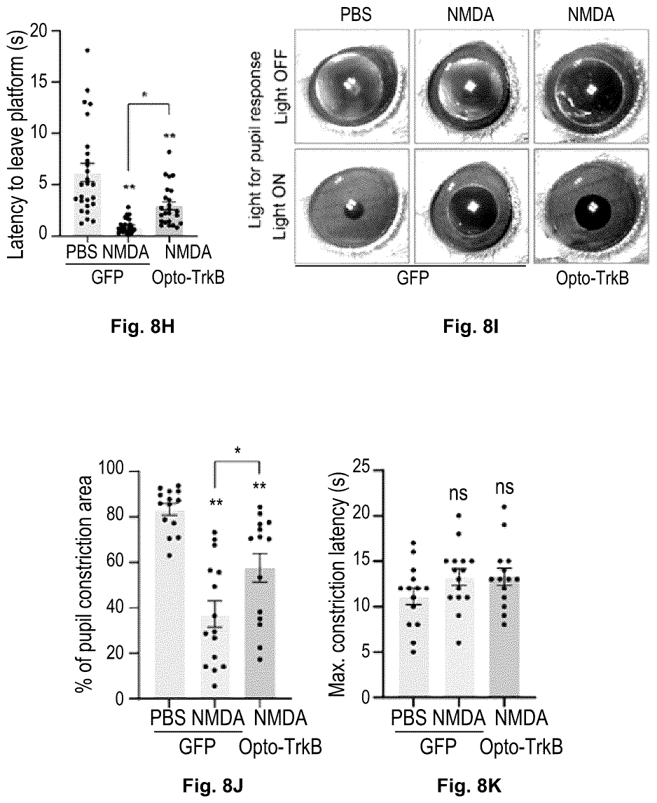

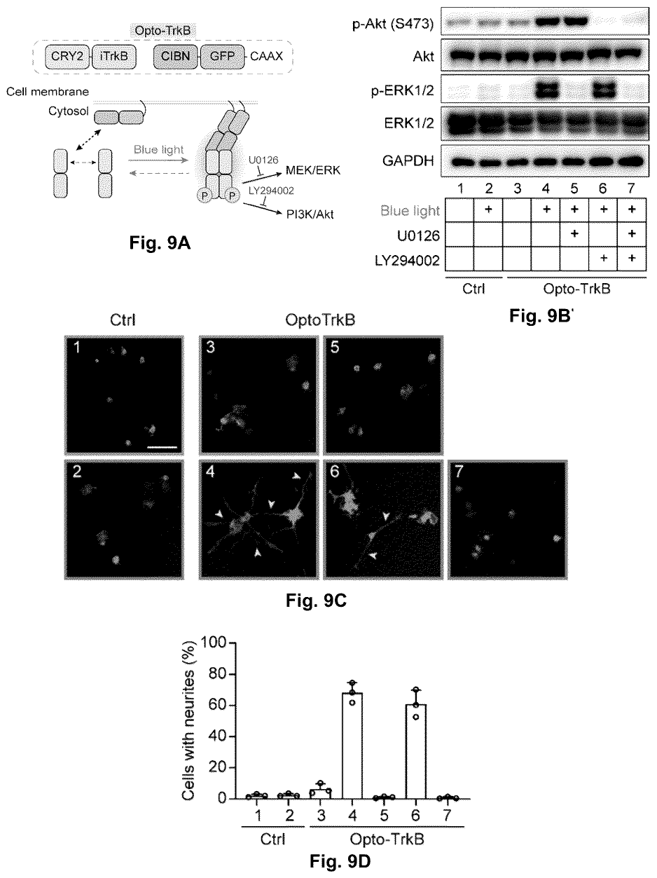

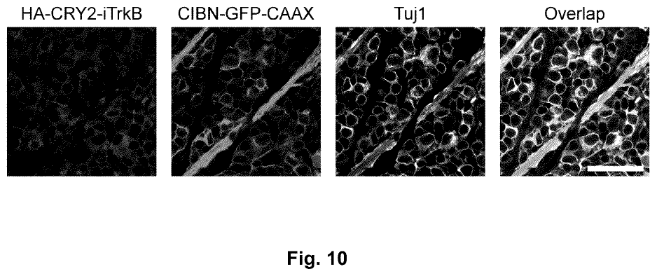

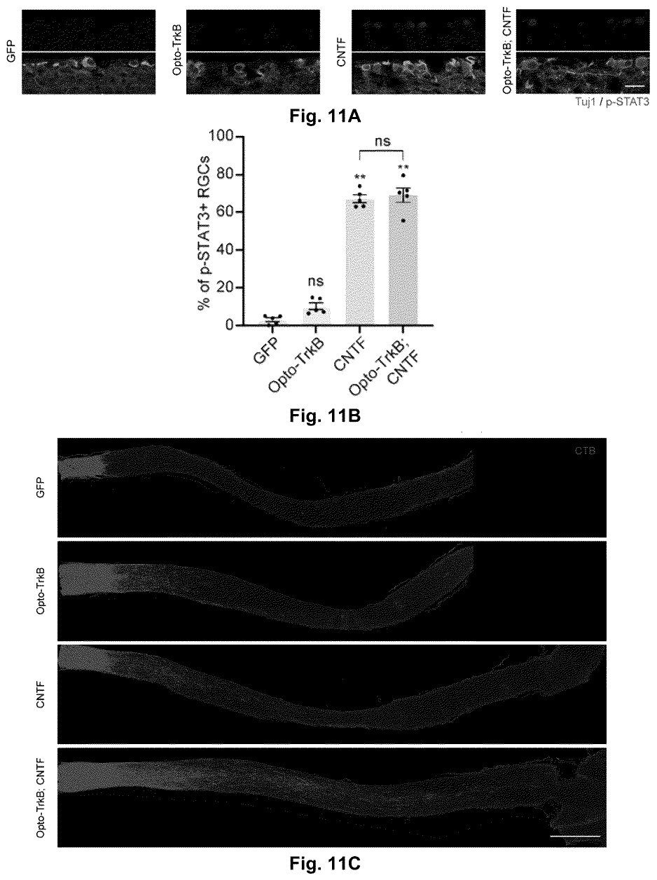

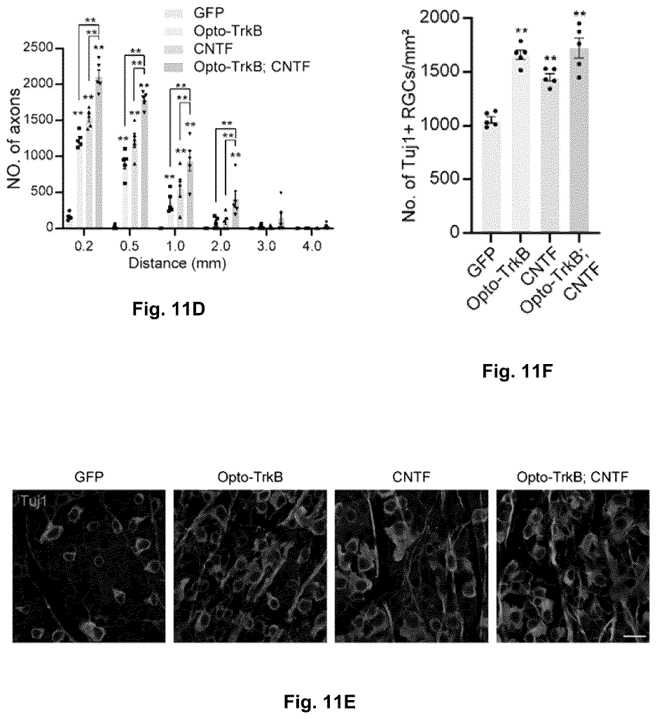

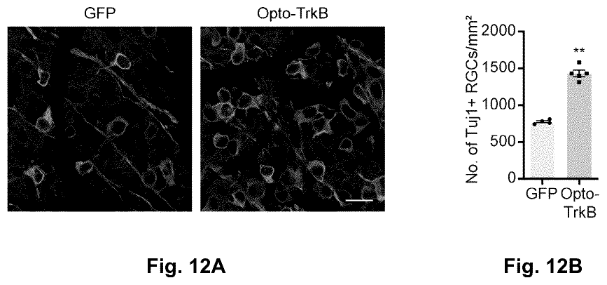

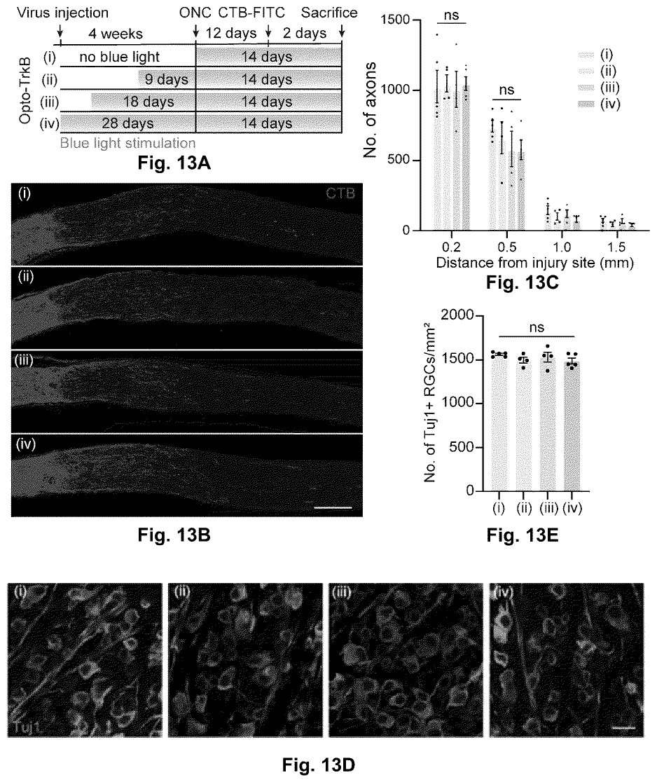

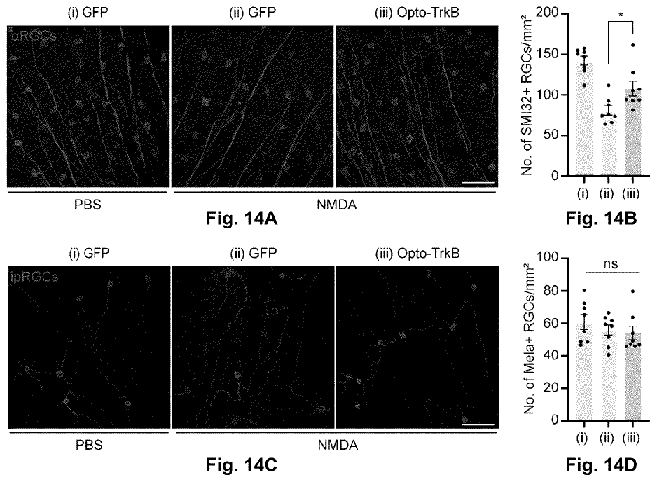

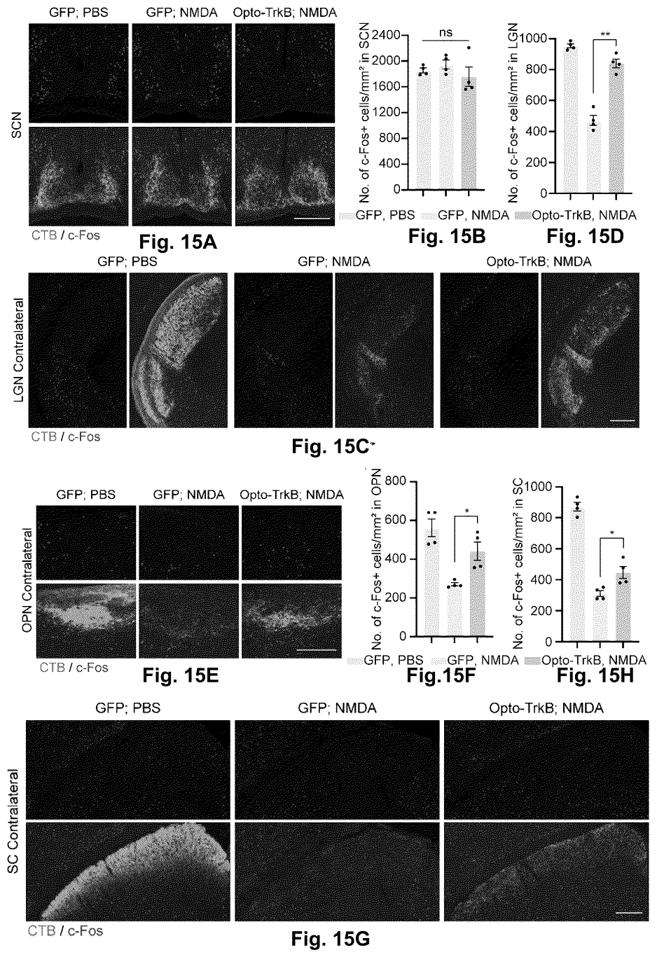

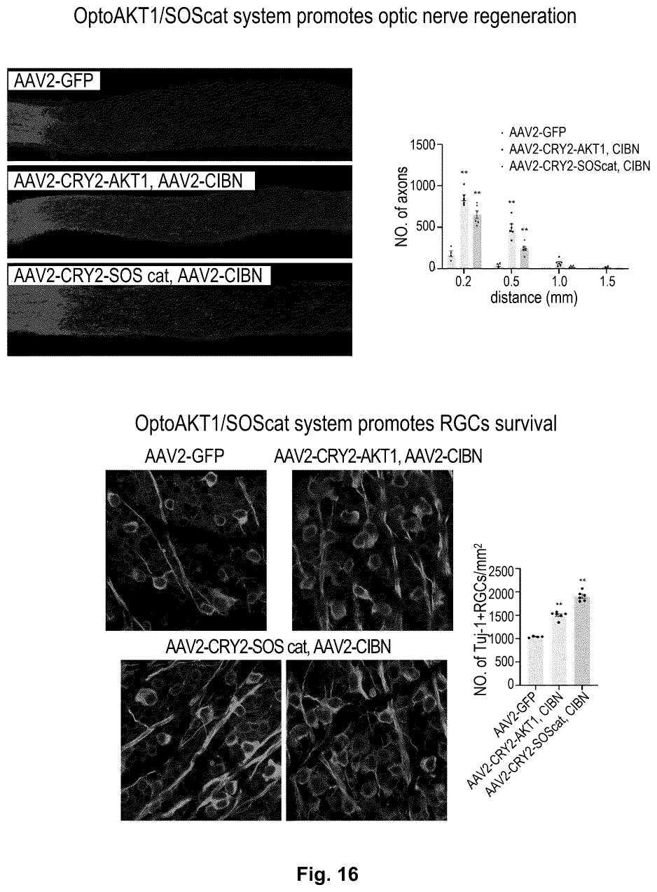

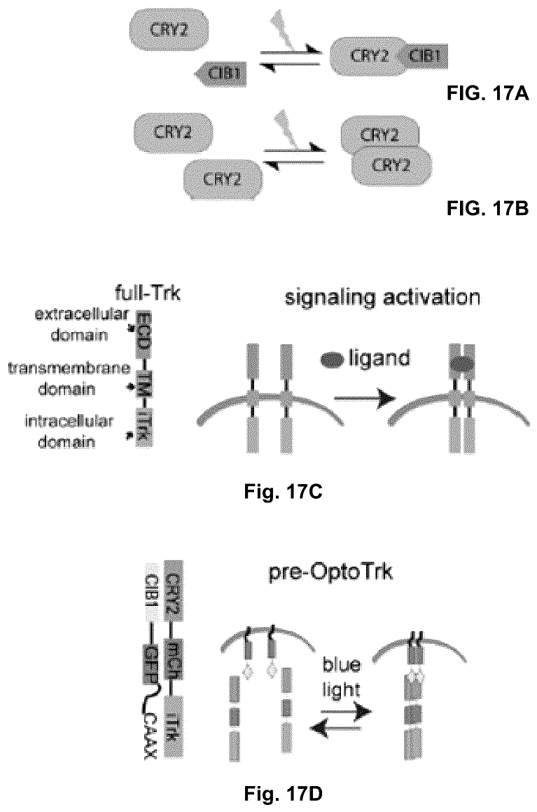

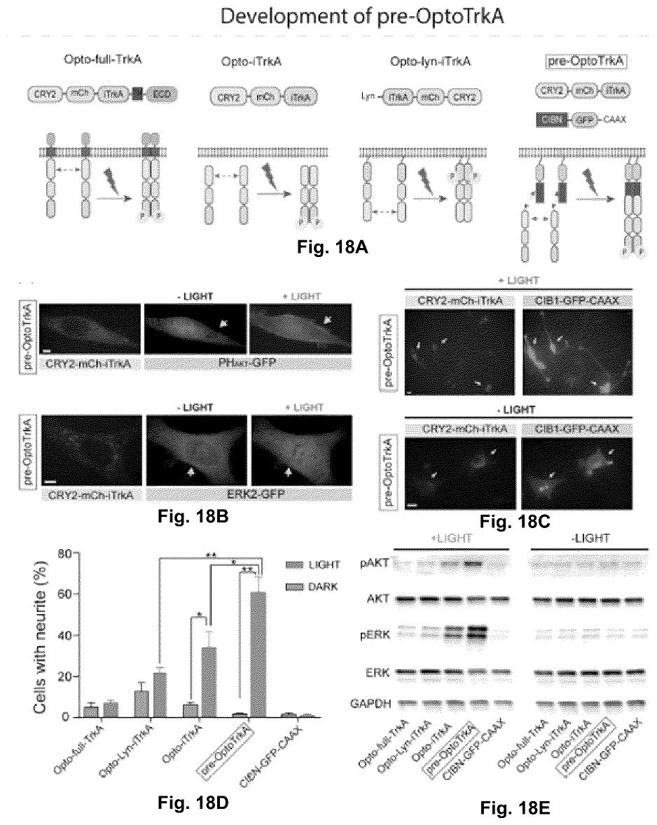

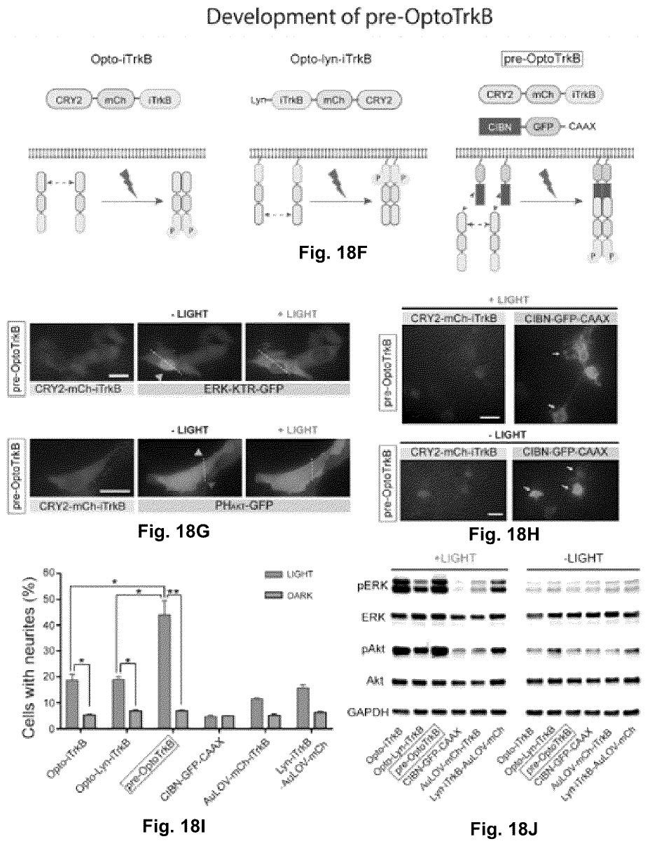

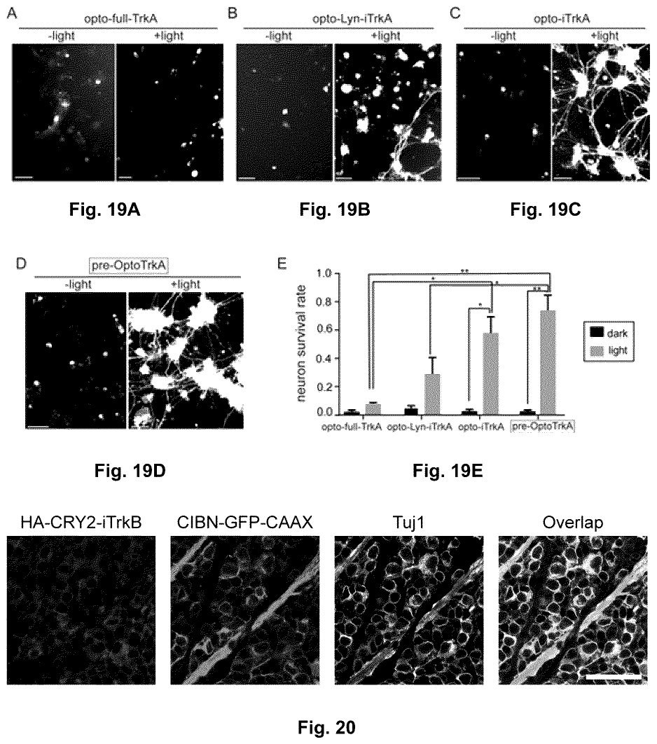

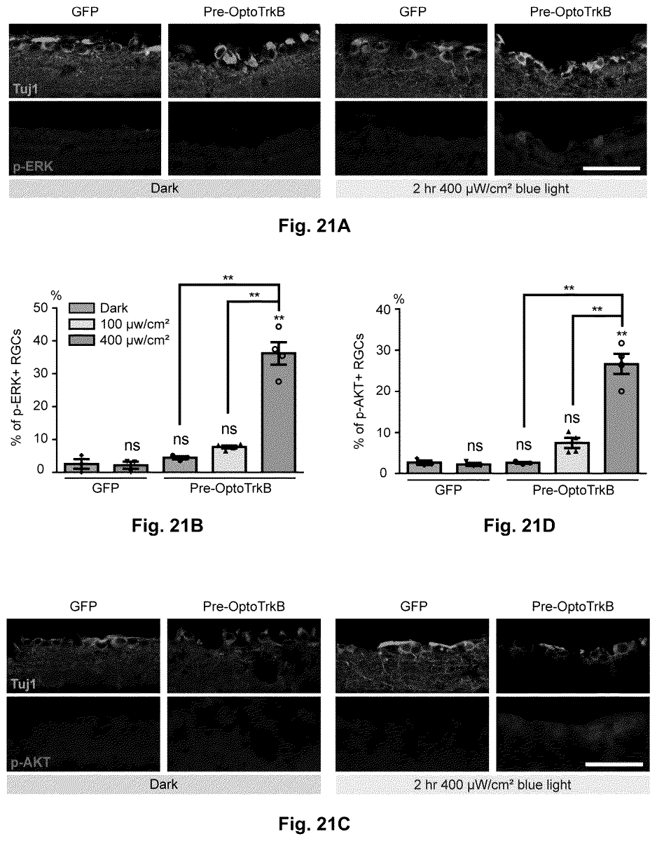

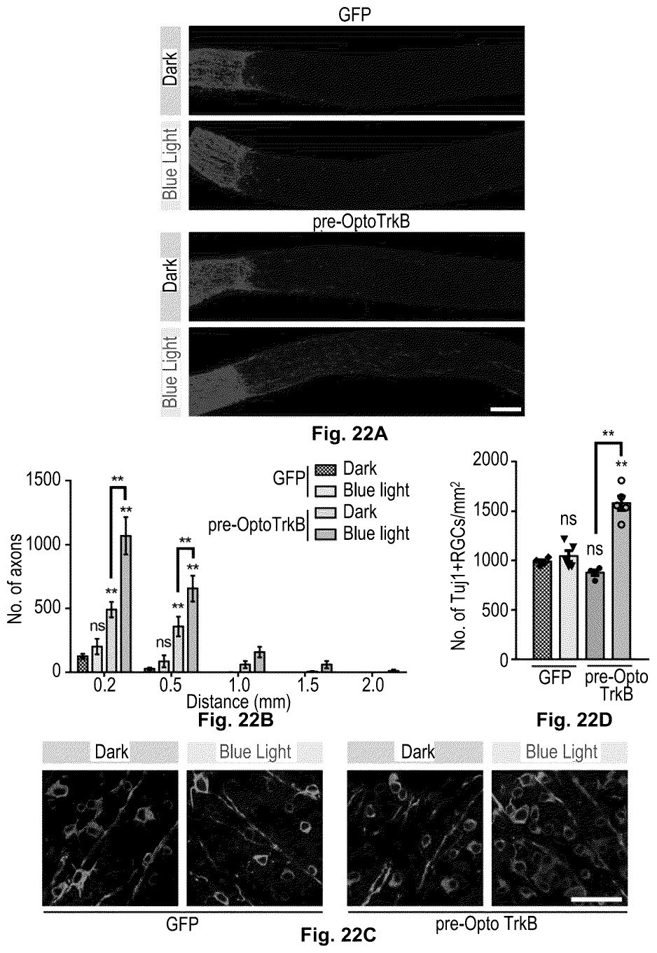

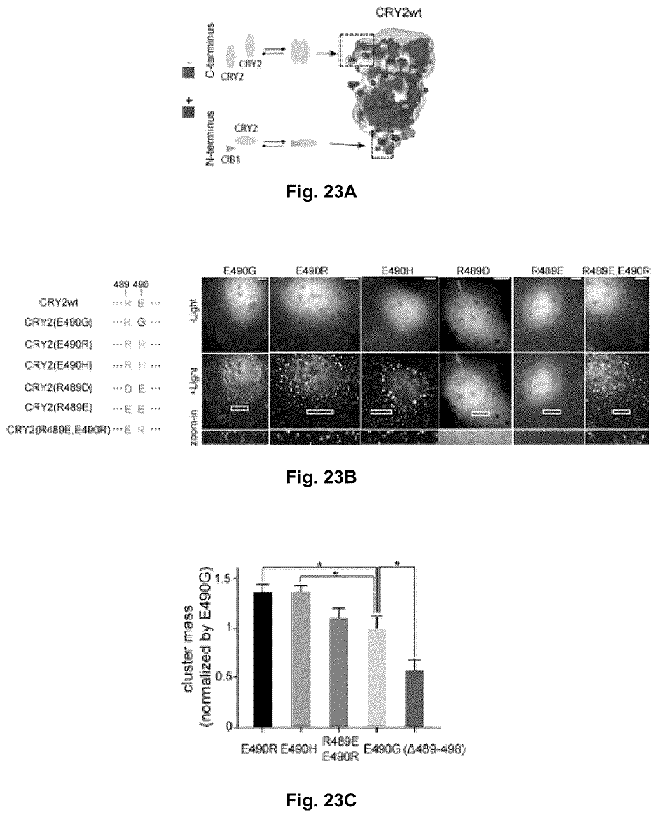

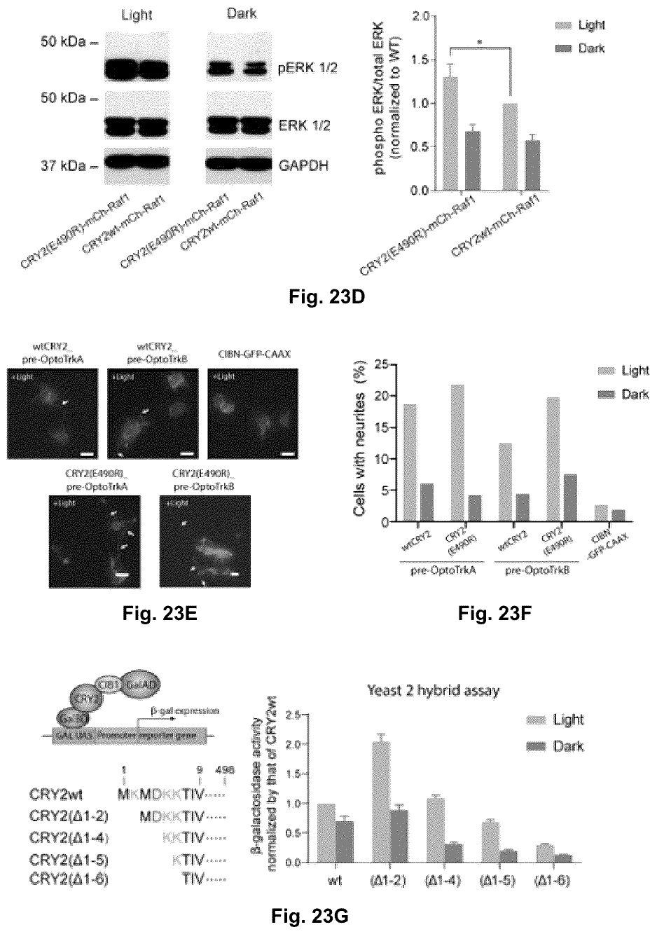

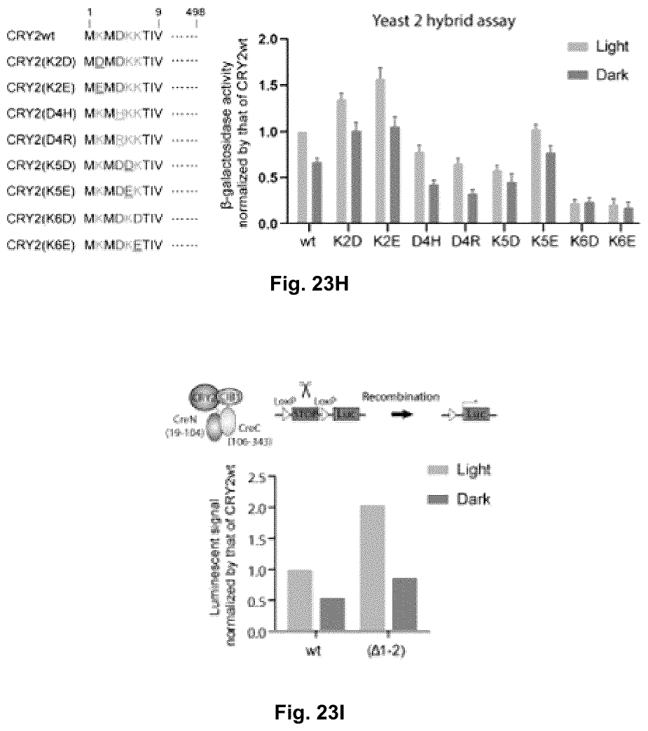

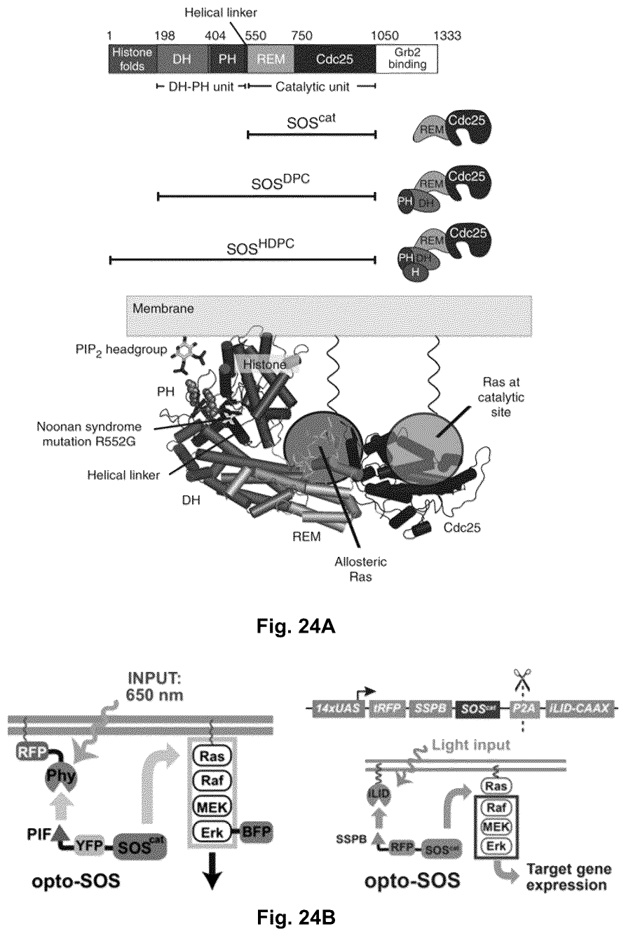

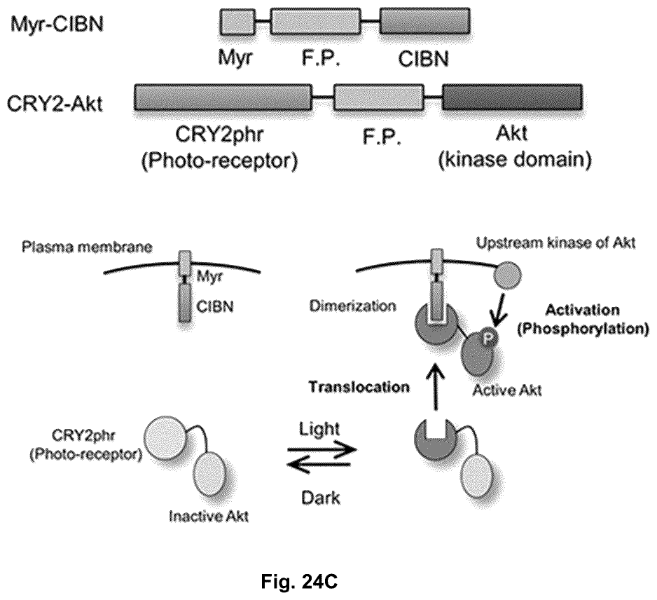

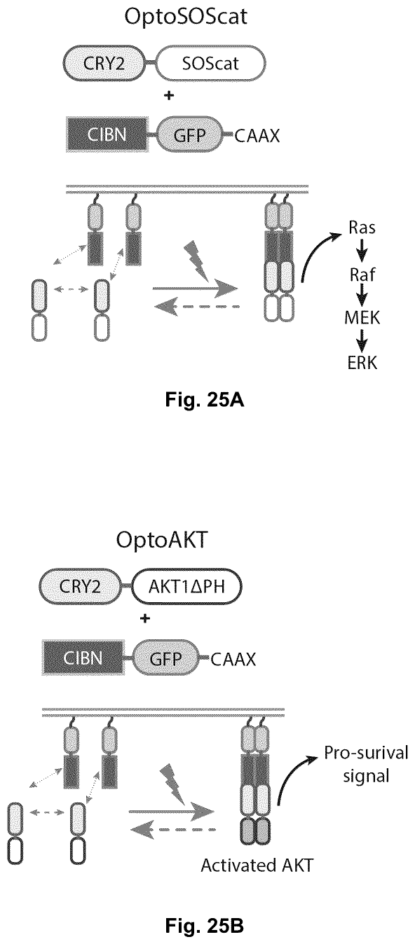

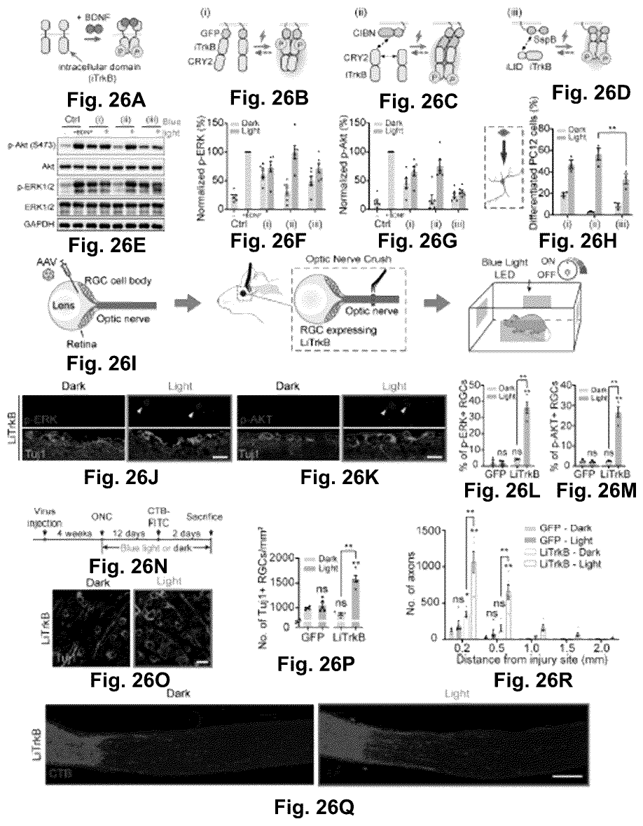

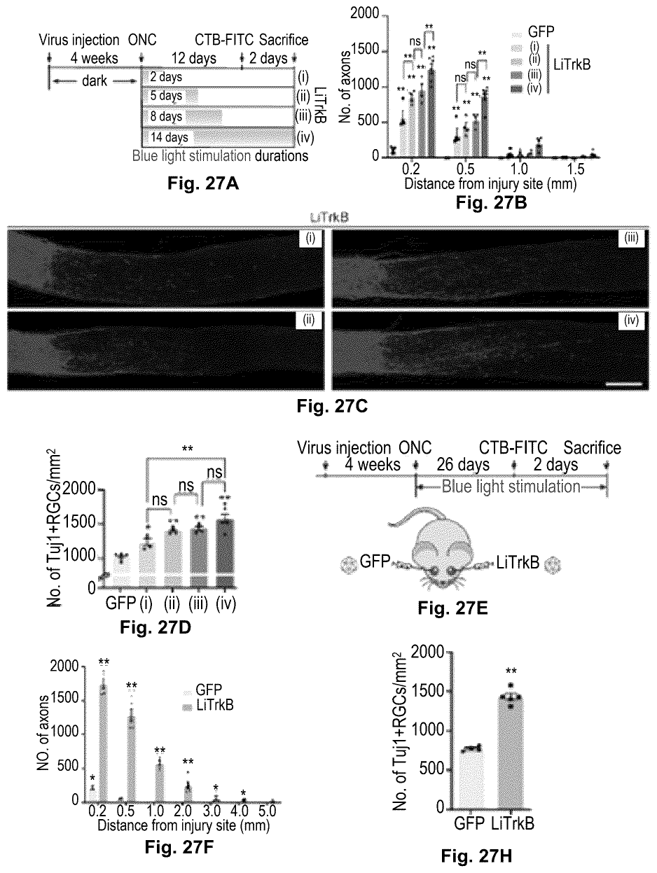

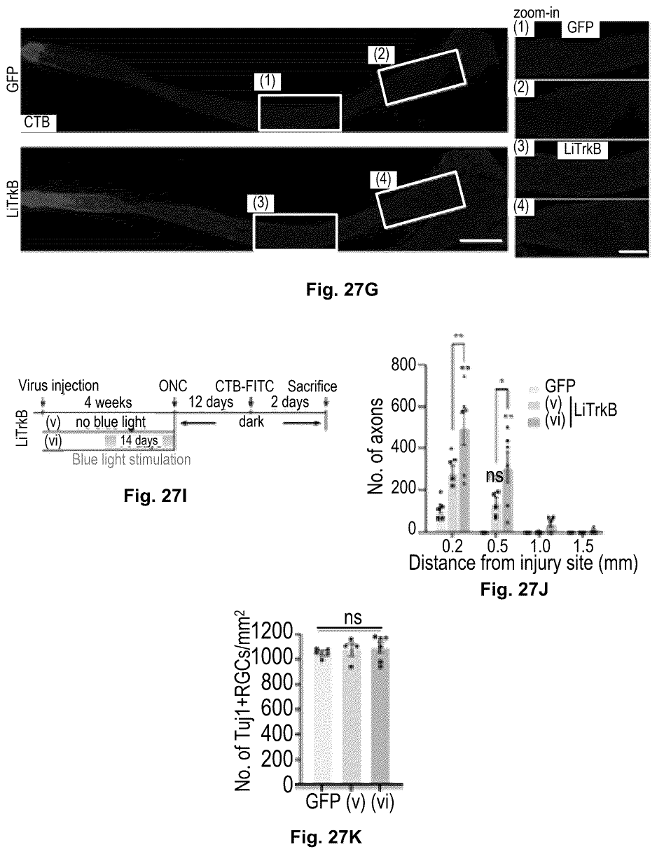

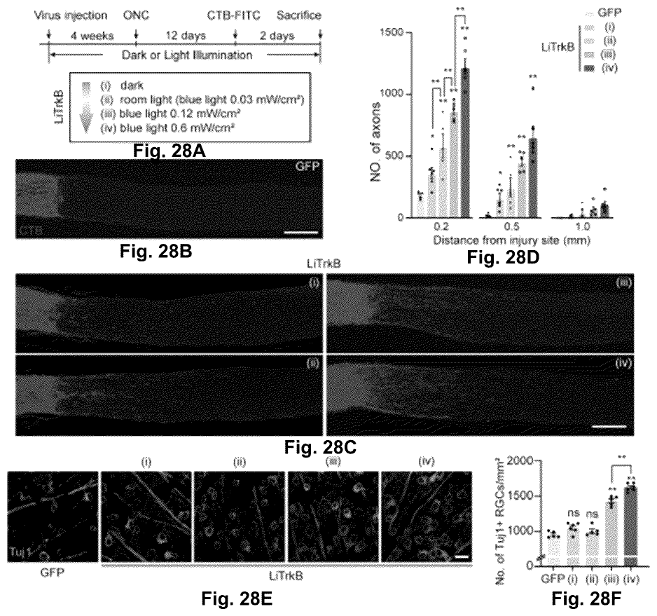

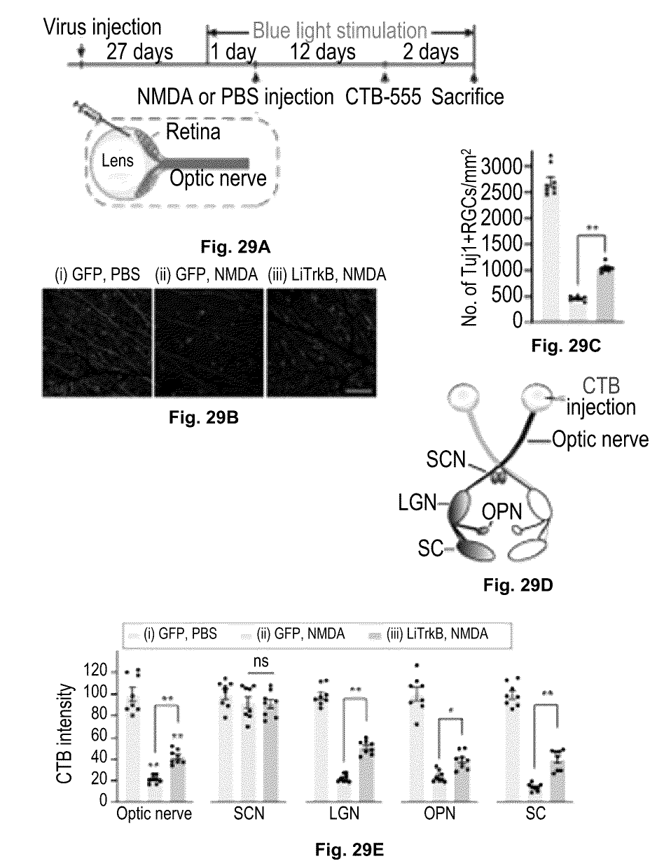

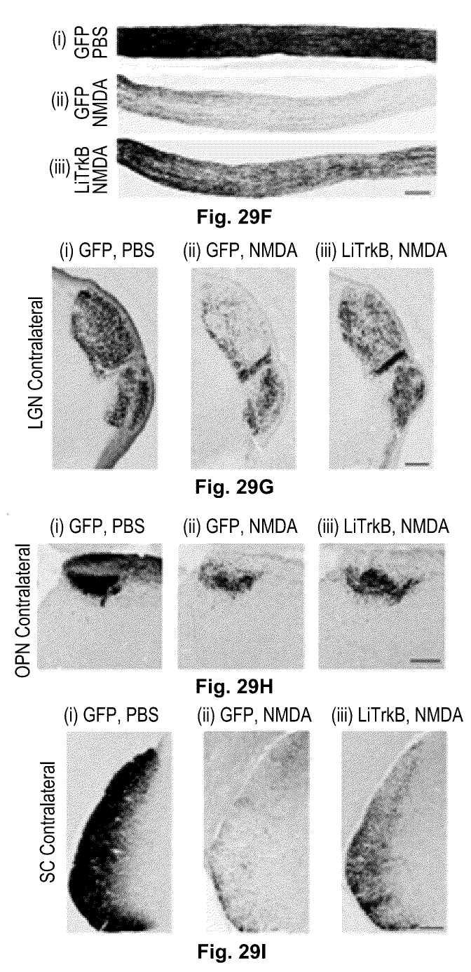

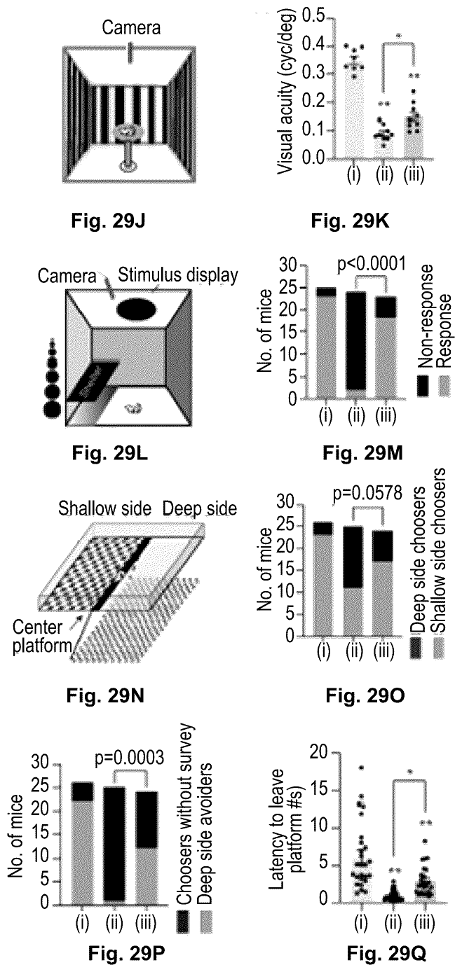

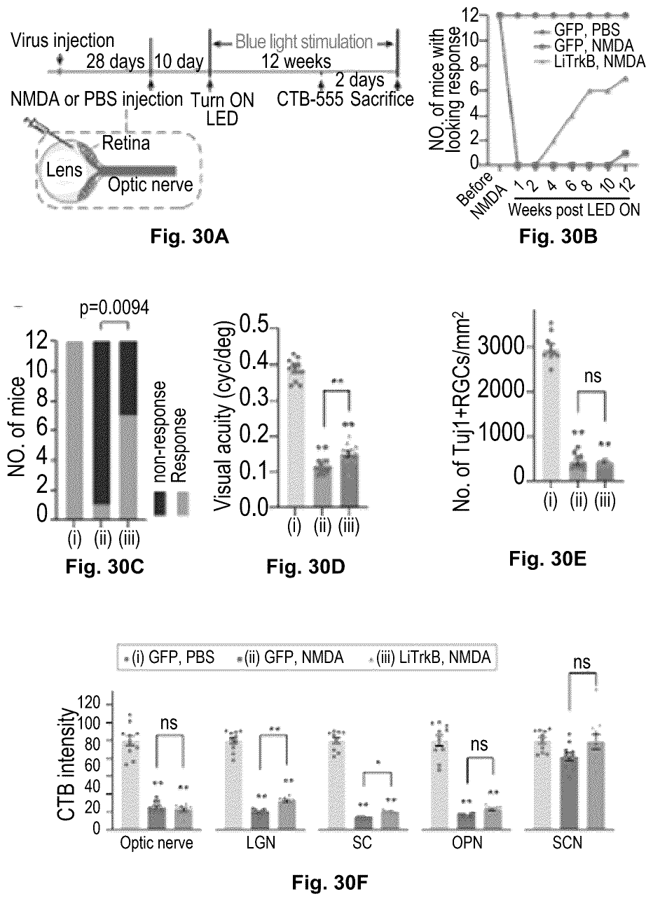

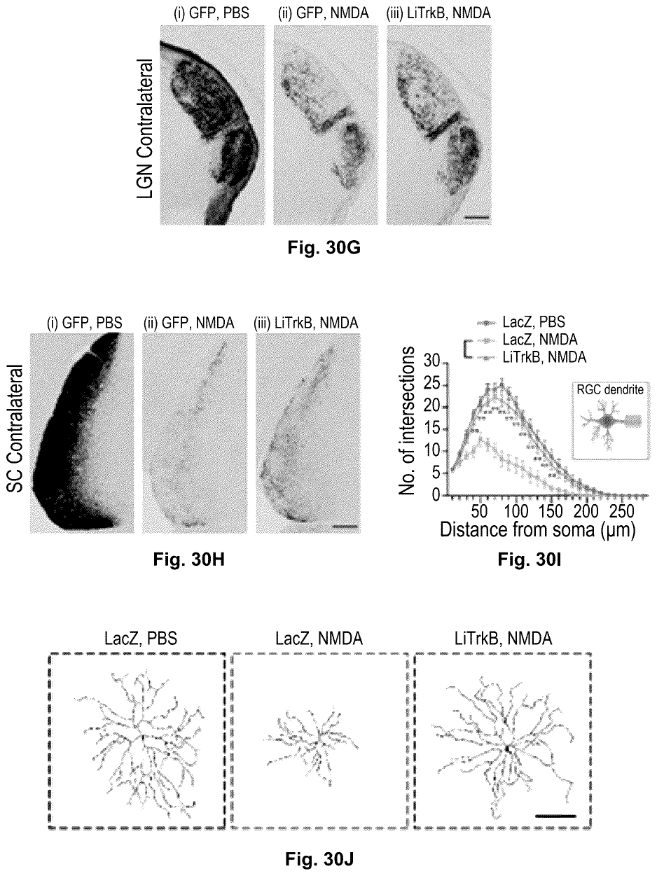

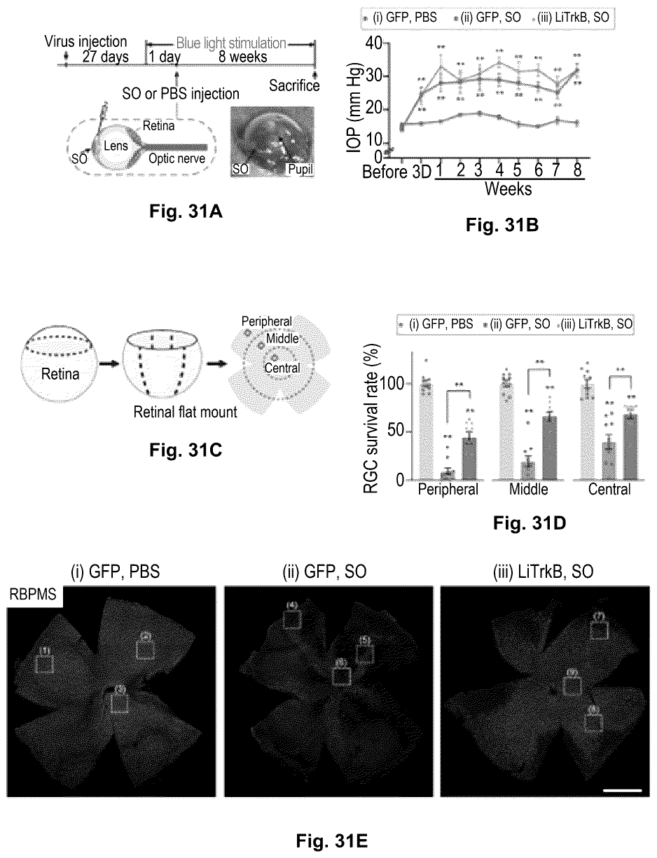

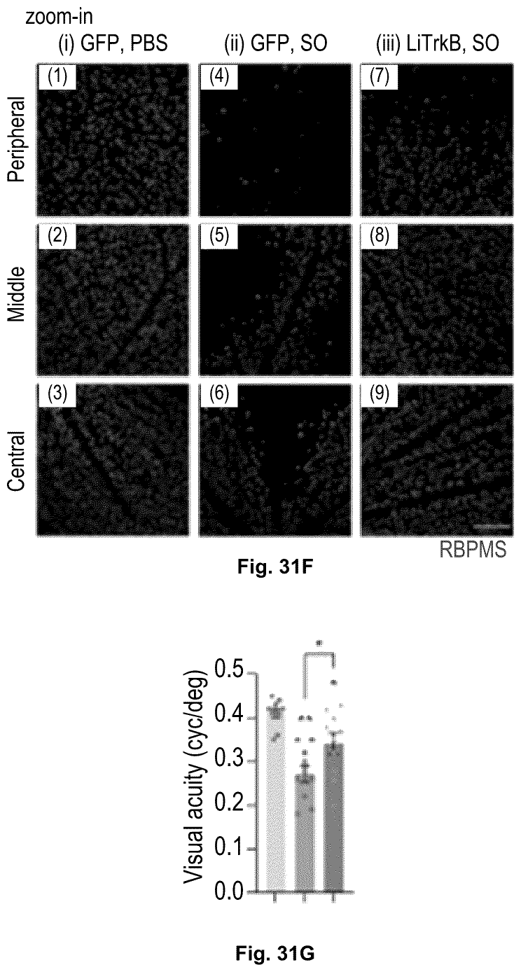

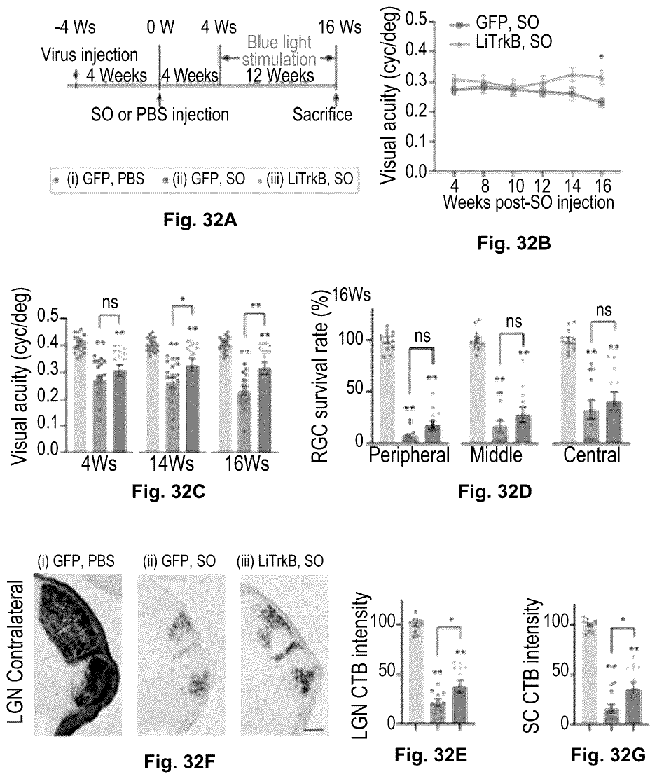

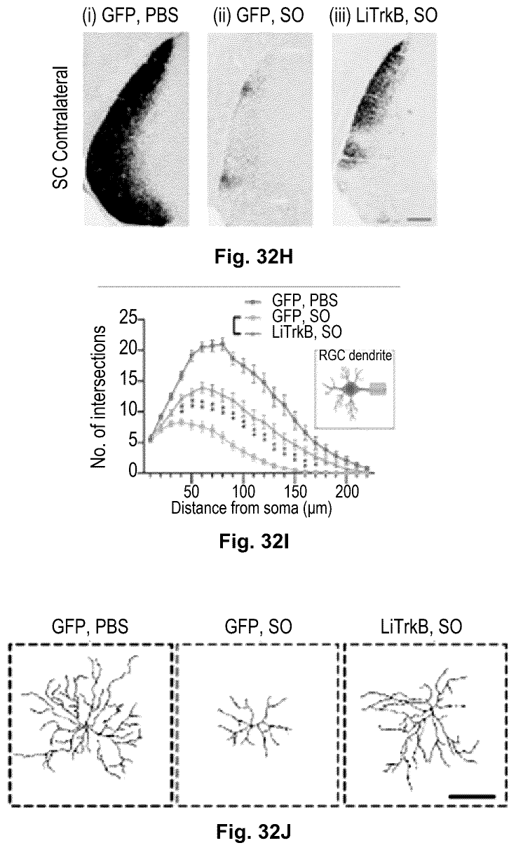

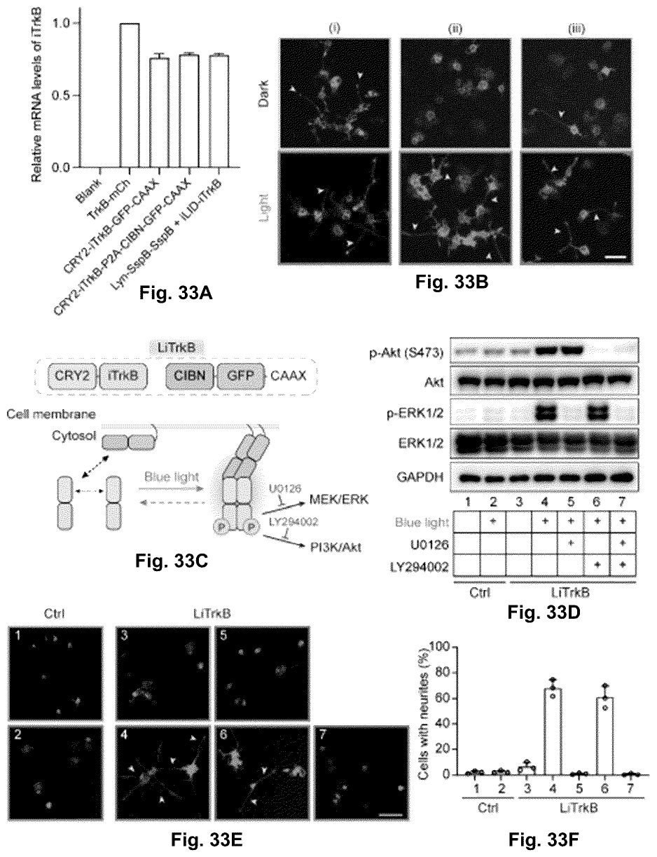

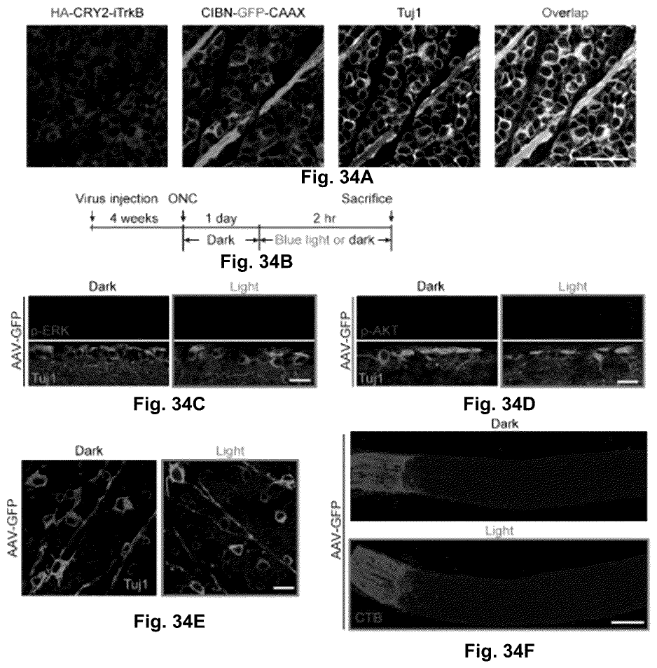

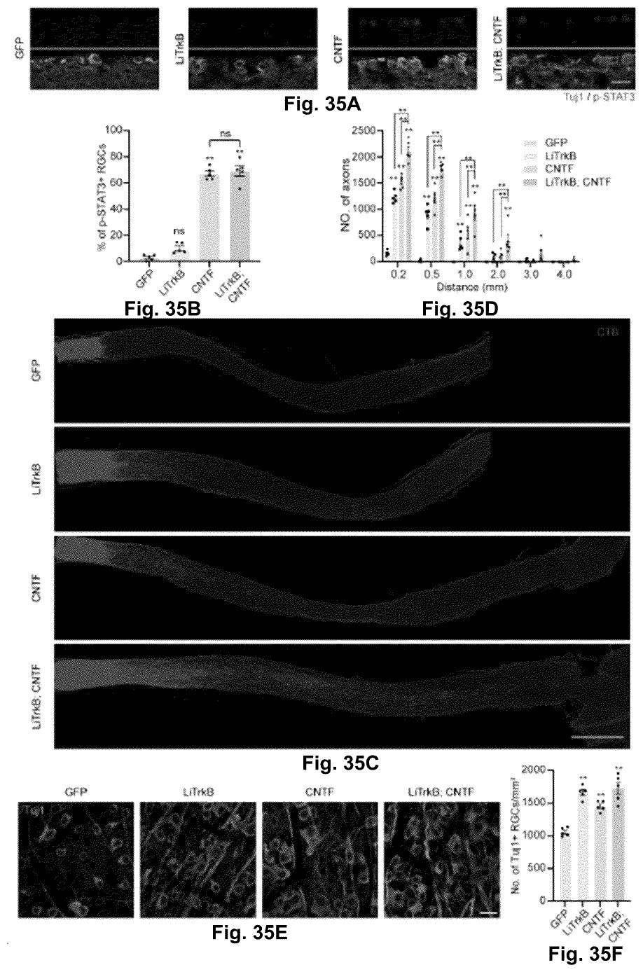

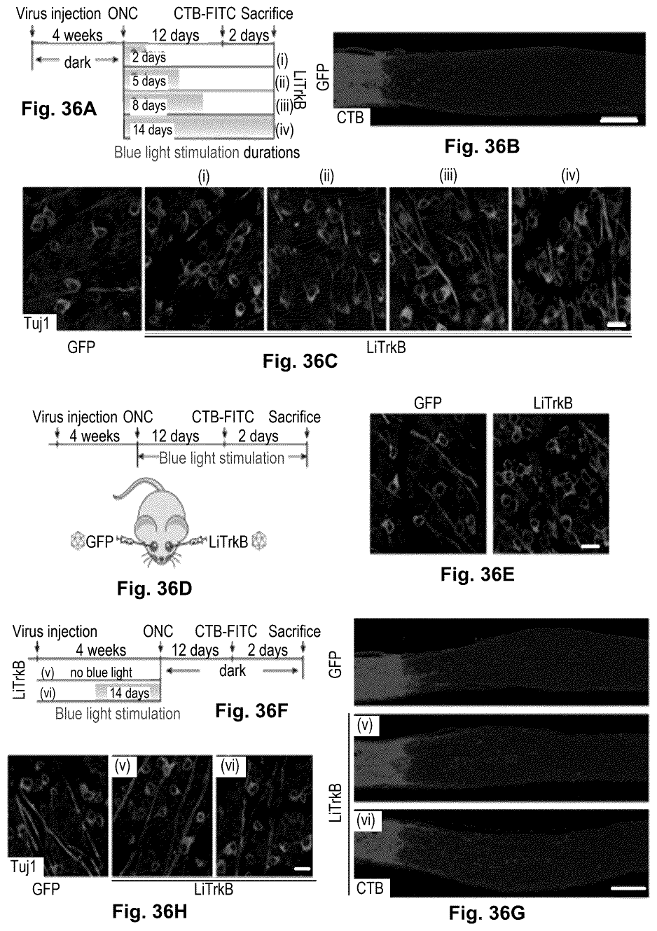

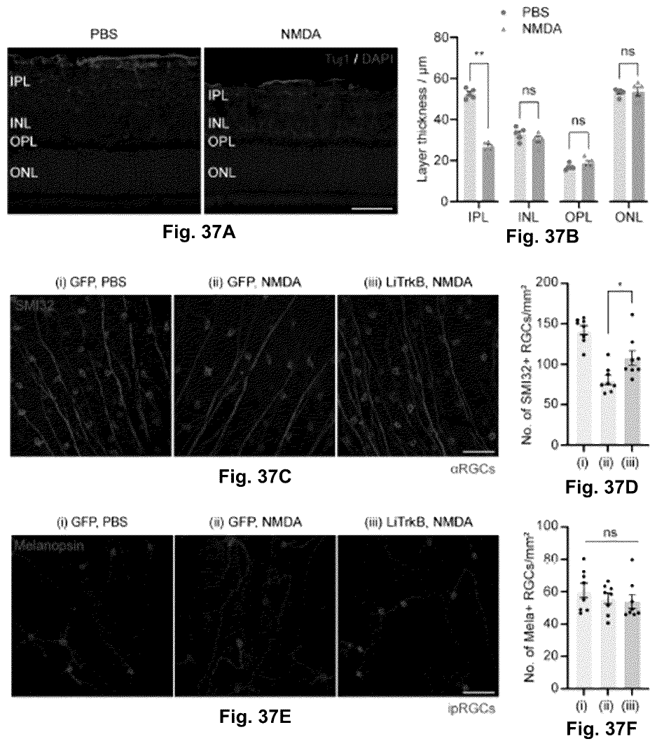

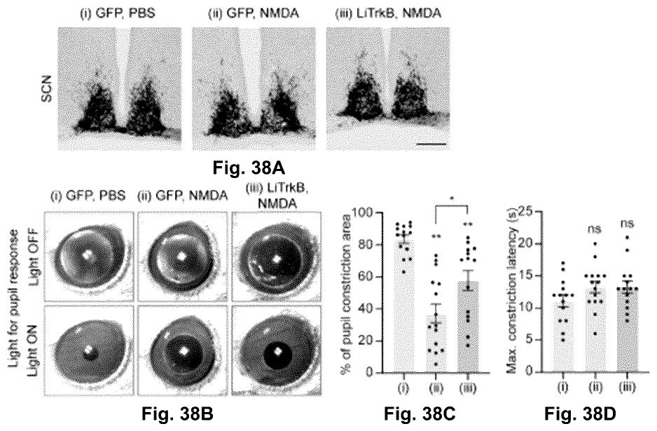

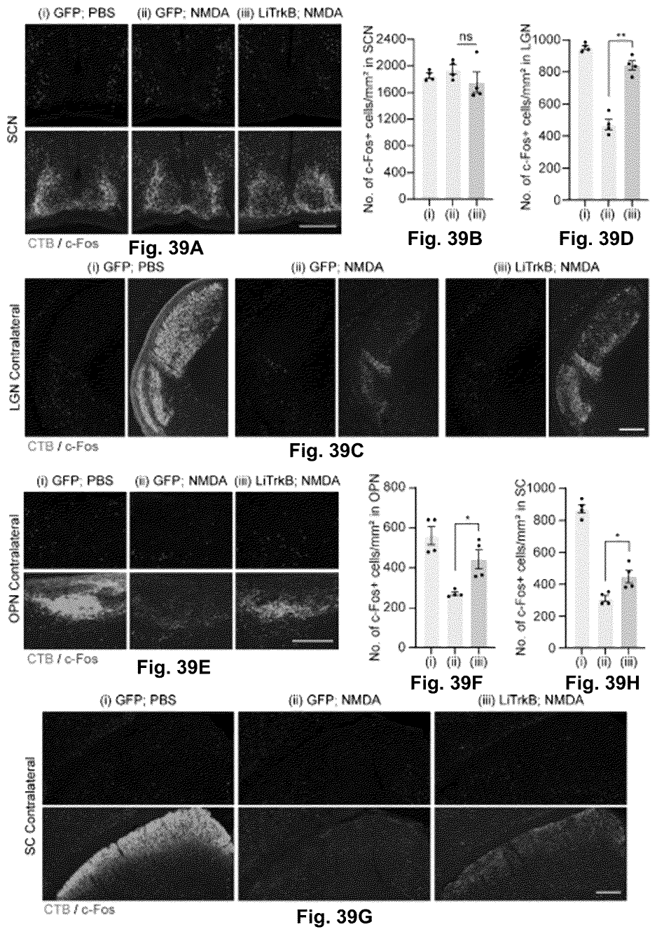

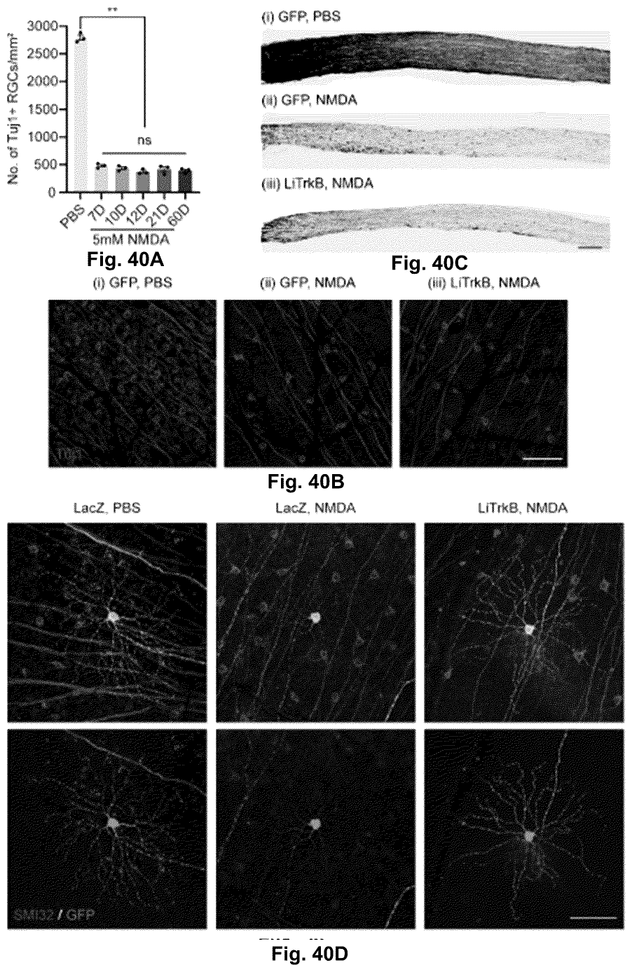

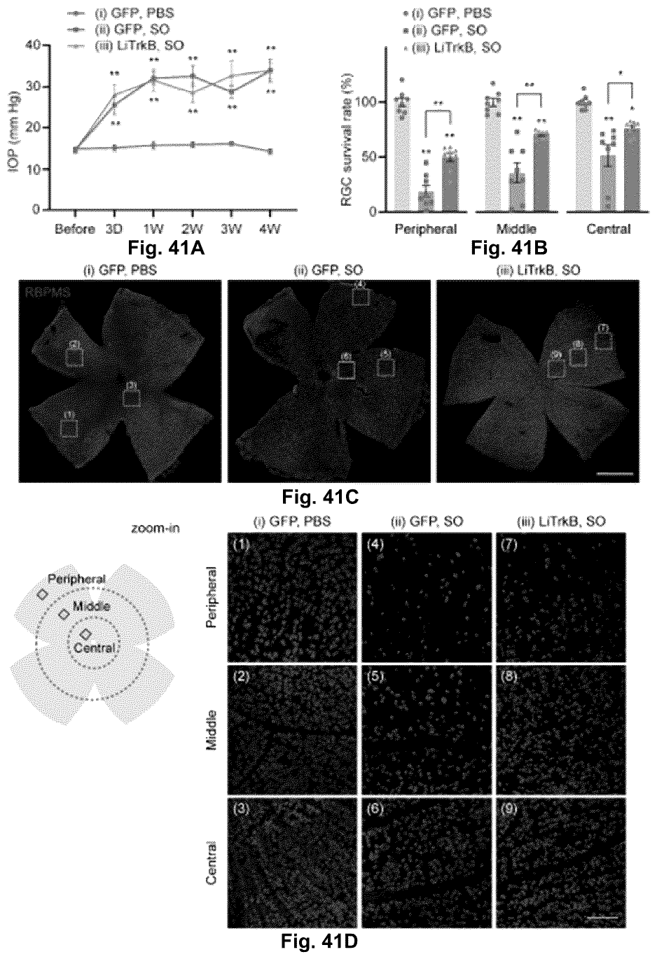

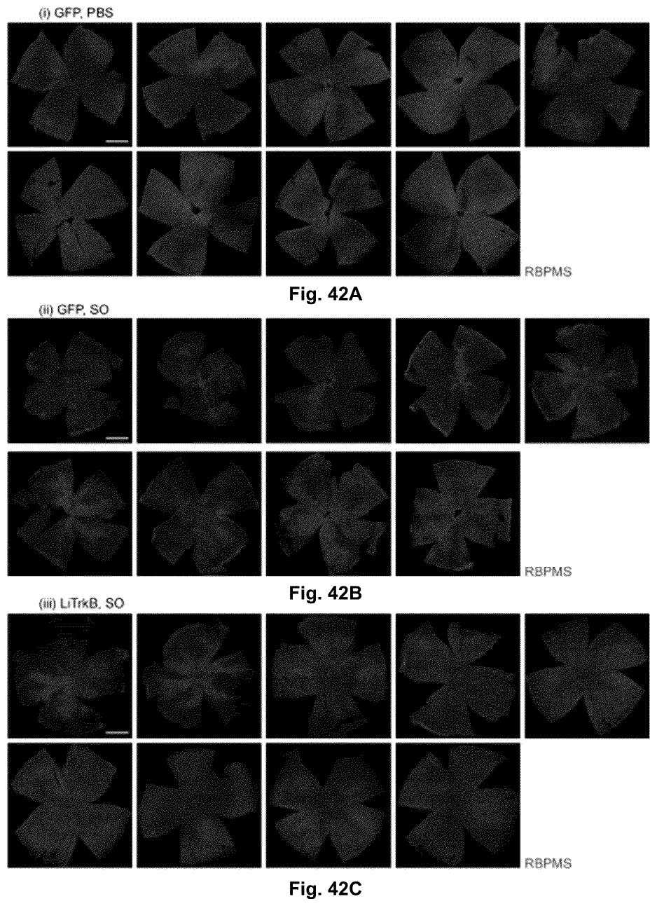

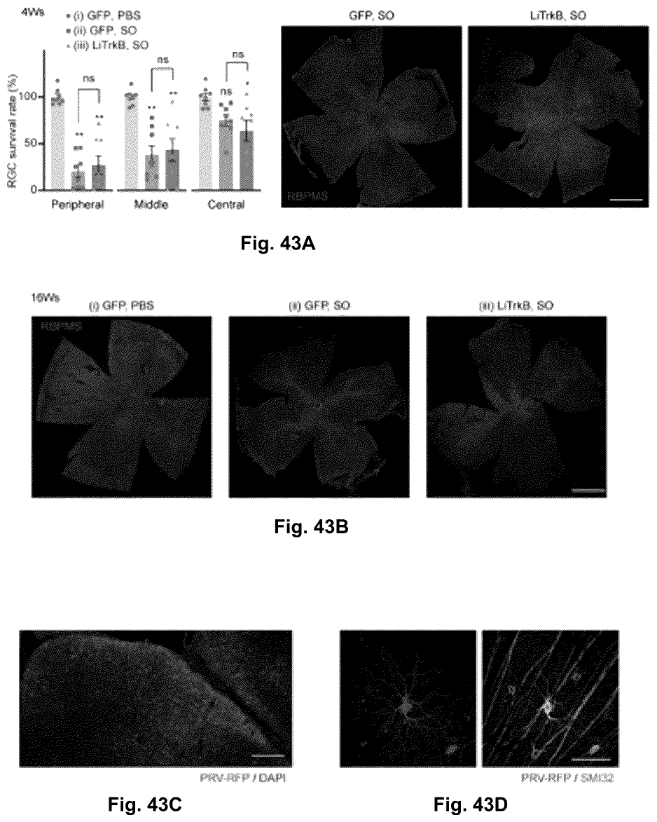

One optogenetic system, which is based on a light-gated protein-protein interaction to control a variety of intracellular processes including gene expression, organelle transport, and signaling transduction, utilizes the Arabidopsis cryptochrome 2 (CRY2) protein, as an optogenetic module that exhibits two types of light-gated interactions (see FIG. 17A-FIG. 17B). Within milliseconds after blue light exposure, CRY2 binds with its binding partner CIB1 (Kennedy et al., Nat Methods 2010, 7 (12), 973-U48), while it can also associate with other CRY2 molecules (Bugaj et al., Nat Methods 2013, 10 (3), 249-252). While a generic concept of this nature was previously disclosed in 2020 publication by Huang et al. (Journal of Molecular Biology 432:3761-3770), the present inventors have made significant improvement in this technology.

### II. Fusion Proteins and their Expression Vectors

### A. General Recombinant Technology

Basic texts disclosing general methods and techniques in the field of recombinant genetics include Sambrook and Russell, Molecular Cloning, A Laboratory Manual (3rd ed. 2001); Kriegler, Gene Transfer and Expression: A Laboratory Manual (1990); and Ausubel et al., eds., Current Protocols in Molecular Biology (1994).

For nucleic acids, sizes are given in either kilobases (kb) or base pairs (bp). These are estimates derived from agarose or acrylamide gel electrophoresis, from sequenced nucleic acids, or from published DNA sequences. For proteins, sizes are given in kilodaltons (kDa) or amino acid residue numbers. Proteins sizes are estimated from gel electrophoresis, from sequenced proteins, from derived amino acid sequences, or from published protein sequences.

Oligonucleotides that are not commercially available can be chemically synthesized, e.g., according to the solid phase phosphoramidite triester method first described by Beaucage & Caruthers, Tetrahedron Lett. 22:1859-1862 (1981), using an automated synthesizer, as described in Van Devanter et. al., Nucleic Acids Res. 12:6159-6168 (1984). Purification of oligonucleotides is performed using any art-recognized strategy, e.g., native acrylamide gel electrophoresis or anion-exchange HPLC as described in Pearson & Reanier, J. Chrom. 255:137-149 (1983).

The sequence of an expression vector, a polynucleotide sequence encoding a polypeptide of interest (e.g., the fusion protein Opto-TrkB having the amino acid sequence SEQ ID NO:3), and synthetic oligonucleotides can be verified after cloning or subcloning using, e.g., the chain termination method for sequencing double-stranded templates of Wallace et al., Gene 16:21-26 (1981).

### B. Fusion Proteins

### One-Protein Scheme

In one aspect of this invention, a fusion protein is created to include an optic-responsive element and a downstream signaling element, so that the optic-responsive element is able to, upon receiving a light signal of an appropriate wavelength in the adequate intensity and duration, activate the downstream signaling element, which is derived from a domain of a signaling molecule through which the signaling molecule activates its downstream effector molecules. As a result of the activation of the downstream signaling element, such as through its dimerization and/or phosphorylation/dephosphorylation of one or more Tyr residues located within the downstream signaling element, the downstream signal transduction pathway originally mediated by a specific signaling molecule in its native environment to further activate a series of downstream molecular and biological events is now controlled by the light signal that ordinarily activates an optically responsive protein such as the wild-type CRY2 protein.

In essence, the present inventors have created a hybrid signal transduction system based on this fusion protein that permits an easy on-and-off control (by a light signal) of a pre-selected downstream signaling pathway determining the occurrence of a series of desirable downstream biological events (such as cellular proliferation, differentiation, migration etc.). In the real-world context, this system allows for a light signal controlled therapeutic scheme in which a medical condition can be treated by the regeneration of a specific cell or tissue type upon light exposure.

The first element of the fusion protein, the optic-responsive element, is a polypeptide or amino acid sequence derived from a light-responsive protein, such as the Arabidopsis thaliana cryptochrome 2 (CRY2) protein, having the amino acid sequence set forth in GenBank Accession No. Q96524 and as SEQ ID NO: 1 herein. The optic-responsive element may be either the full-length wild-type CRY2 protein, or a mutant full-length CRY2 protein comprising any one or two or more of the mutations independently selected from the group consisting of: (1) a deletion of the first and second amino acids of N-terminus of CRY2 (Δ1/2); (2) a K2S substitution; (3) an E281A substitution; and (4) an E490R substitution. In other cases, the optic-responsive element may be a truncated version of the CRY2 protein, for instance, comprising the CRY2 protein photolyase homology (PHR) domain, i.e., the 1-498 segment of SEQ ID NO: 1 but less than full-length of SEQ ID NO:1, e.g., no more than about 550 or about 600 amino acids in length.

The second element of the fusion protein, the downstream signaling element, is a polypeptide or amino acid sequence derived from a protein capable of transducing cellular signaling through a pathway leading to desired cellular events, such as the regeneration of certain cell/tissue type(s), resulting in alleviation of clinical symptoms of certain diseases or conditions involving the loss or diminished biological functions in the related cells and tissue, e.g., in sensory perception (such as vision, hearing, smell, or taste, etc.) and neurological or physical functions (such as memory or motion skills). Thus, the downstream signaling element can be taken from virtually any signal transduction molecules (e.g., receptor type of proteins) capable of directly interacting with its signaling molecule (e.g., one of its natural ligands) and subsequently activating at least one effector molecule to trigger a series of further downstream cellular events. Typically, the downstream signaling element is a portion of a receptor type of protein responsible for intracellular activation upon binding to its native signaling molecule (e.g., a ligand) and subsequent activation of further downstream effector molecules, such as the intracellular domain of a receptor-type of protein (e.g., the intracellular domain of epidermal growth factor receptor (EGFR), fibroblast growth factor receptor (FGFR), or a receptor tyrosine kinase (RTK) such as tropomyosin receptor kinase A/B/C (TrkA/B/C), especially TrkB). One example of the downstream signaling element for the fusion protein comprises the intracellular domain of TrkB (iTrkB), corresponding to the 455-822 segment of the full-length rat TrkB amino acid sequence set forth in GenBank Accession No. Q63604 and as SEQ ID NO:2 herein, or the underline portion in SEQ ID NO: 3). Another example of the downstream signaling element for the fusion protein comprises the intracellular domain of human TrkB protein, which has the amino acid sequence set for in SEQ ID NO:7 (the underline portion being human iTrkB). The extracellular domain of the signaling/receptor protein is not included in the fusion protein, and the transmembrane domain of the signaling/receptor protein is usually excluded as well.

Other exemplary downstream signaling elements of the fusion protein may comprise a domain taken from and responsible for the activity of a signaling molecule for its role in a signal transduction pathway such as SOS/Ras signaling, AKT signaling, and GDNF signaling, for example, the kinase domain of an AKT1 protein (the 128-480 segment of the protein amino acid sequence as set forth in Uniprot P31749 AKT1_HUMAN or P31750 AKT1_MOUSE) or the catalytic domain of the signaling molecule known as the SOS protein (SOScat, the 550-1050 segment of human SOS protein amino acid sequence set forth in GenBank Accession No. Q07890).

In some cases, the fusion protein has the optic-responsive element fused to the downstream signaling element, placed in this order from the N-terminus to the C-terminus of the fusion protein. Optionally, a peptide linker is used to connecting two elements immediately adjacent to each other. A typical linker is about 2-20, 3-15, 5-10, or about 6-8 amino acids in length, often no longer than 5, 6, 7, 8, 9, 10, 11, 12, 13, 14, 15, 16, 17, 18, 19, or 20 amino acids.

For the sake of ease of application, it is desirable to have the fusion protein of this invention located at or near the membrane of a host cell such that light signaling can be most effectively administered and received by the optic-responsive element of the fusion protein. To this end, the fusion protein of this invention may further include a third amino acid sequence, a cell membrane-targeting element, which serves to direct the fusion protein to be localized to the close proximity of cell membrane. Exemplary cell membrane-targeting elements include a Lyn tag and a CAAX motif. Typically, such a cell membrane-targeting element is located at the N- or C-terminus of the fusion protein.

Another optional element that might be present in certain versions of the fusion protein is an element that can provide a detectable signal (e.g., mCherry or GFP). This detectable moiety allows for easy detection and confirmation of the presence of the fusion protein in a host cell.

### Two-Protein Scheme

Alternatively, the fusion protein of the present invention does not comprise any cell membrane-target element to place the fusion protein at or near the host cell membrane. Rather, the fusion protein relies on a second fusion protein to direct it to localize at or near the cell membrane. This second fusion protein, by virtue of its comprising a cell membrane-targeting element and a CRY2-binding element, acts to recruit the first fusion protein of this invention (which comprises an optic-responsive element and a downstream signaling element) to the host cell membrane.

One exemplary membrane-targeting element comprises the CAAX motif. One exemplary CRY2-binding element is an N-terminal fragment of the basic helix-loop-helix Arabidopsis protein CIB1 (CIBN) comprising the 1-170 segment of the CIBN but no longer than 200 or 250 amino acids. In some cases, the membrane-targeting element and the CRY2-binding element are present in this order from the N-terminus to the C-terminus of the second fusion protein. In other cases, the CRY2-binding element and the membrane-targeting element are present in this order from the N-terminus to the C-terminus of the second fusion protein. Optionally, a third element, a detectable moiety permitting easy detection of the second fusion protein in a host cell, is also present in the second fusion protein. For example, an mCherry or GFP may be included and located in the middle of the second fusion protein between the membrane-targeting element and the CRY2-binding element.

For either one or both of the fusion proteins in both one-protein and two-protein schemes described above and herein, an optional peptide linker may be used to connect any two adjacent elements. A typical linker is about 2-20, 3-15, 5-10, or about 6-8 amino acids in length, often no longer than 5, 6, 7, 8, 9, 10, 11, 12, 13, 14, 15, 16, 17, 18, 19, or 20 amino acids.

### B. Recombinant Expression of the Fusion Proteins

A first fusion protein of this invention comprising an optic-responsive element and a downstream signaling element, optionally with a second fusion protein comprising a cell membrane-targeting element and a CRY2-binding element, can be produced in a desired target host cell using routine techniques in the field of recombinant genetics, relying on the polynucleotide sequences, especially expression vector(s), encoding the fusion proteins disclosed above and herein.

To obtain high level expression of a nucleic acid encoding a desired polypeptide, one typically subclones a polynucleotide encoding the polypeptide into an expression vector that contains a strong promoter to direct transcription, a transcription/translation terminator and a ribosome binding site for translational initiation. Suitable bacterial promoters are well known in the art and described, e.g., in Sambrook and Russell, supra, and Ausubel et al., supra. One exemplary eukaryotic expression vector is an adenoviral vector, an adeno-associated vector, or a retroviral vector.

Standard transfection methods can be used to produce mammalian cells, especially targeted human cells that express sufficient quantities of a recombinant polypeptide (e.g., a first fusion protein, optionally with a second fusion protein). Transformation of eukaryotic cells are performed according to standard techniques (see, e.g., Morrison, J. Bact. 132:349-351 (1977); Clark-Curtiss & Curtiss, Methods in Enzymology 101:347-362 (Wu et al., eds, 1983). Any of the well-known procedures for introducing foreign nucleotide sequences into host cells may be used, although various forms of expression vectors, especially viral vectors, may be preferred (see, e.g., Sambrook and Russell, supra).

In particular, viral vector systems are useful in the introduction and expression of a fusion protein of this invention. They include naturally occurring or recombinant viral vector systems. Depending upon the particular application, suitable viral vectors include replication competent, replication deficient, and conditionally replicating viral vectors. For example, viral vectors can be derived from the genome of human or bovine adenoviruses, vaccinia virus, herpes virus, adenovirus, adeno-associated virus (AAV), minute virus of mice (MVM), sindbis virus, and retroviruses (including but not limited to Rous sarcoma virus), and Moloney murine leukemia virus (MoMLV). Typically, the coding sequence for the fusion protein is inserted into such vectors to allow packaging of the gene construct, often with accompanying viral DNA, followed by infection of a host cell and expression of the protein in the cell.

Similarly, viral envelopes used for packaging gene constructs that include the coding sequence for the fusion protein can be modified by the addition of receptor ligands or antibodies specific for a receptor to permit receptor-mediated endocytosis into specific host cells being targeted for recombinant expression of the fusion protein (see, e.g., WO 93/20221, WO 93/14188, and WO 94/06923).

Once the expression vector coding a recombinant polypeptide, e.g., a fusion protein of this invention, is introduced into the target host cells, its presence and quantity may be further verified by methods such as immunoassays (e.g., Western blot or ELISA), mass spectrometry, or by way of detecting a signal from a detectable moiety contained in the fusion protein, such as mCherry or GFP.

### III. Methods of Use

The present inventors have devised a strategy to exert easy and efficient control of signal transduction pathways and therefore regulation of various cellular processes such as cell proliferation, differentiation, survival, migration, regeneration, and the like: through the use of a genetically engineered upstream signaling molecule, a hybrid protein containing an optic-responsive element and a downstream signaling element, that directly responds to a light signal rather than the binding of an extracellular ligand, they are able to conveniently regulate these cellular events through light exposure of host cells expressing the engineered hybrid protein. By regulating the pertinent cellular events, such as promoting proliferation and regeneration, in the pertinent cell and tissue type, the engineered hybrid signaling protein can be used for alleviating symptoms of diseases and conditions involving damaged or lost biological functions including sensory perception (e.g., vision, hearing, etc.) and neurological or physiological activities.

The method of this invention allows for activation of a signal transduction pathway in a cell by delivering an optic signal to the cell. The cell may be present in a culture, e.g., the method is for in vitro studies. Or the cell may be present in the body of a subject, e.g., the method is performed for in vivo studies or medical treatment. In general, a target cell in the method of this invention is first introduced an expression vector encoding a fusion protein comprising an optic-responsive element and a downstream signaling element such that the targeted cell now expresses the fusion protein, which is capable of activating a pre-selected downstream signaling pathway and desired cellular events upon light exposure within a pre-determined wavelength range.

Depending on the study goal or the disease/condition being treated, activation of the downstream signal transduction pathway results in proliferation of the target cell, or survival and/or regeneration of the target cell. Similarly, a variety of cell types may be suitable for use in the claimed method. In some cases, when the method is practiced for restoration of neurological functions, a neuronal cell (e.g., PC12 cell for in vitro study) may be used. In other cases, when the method is practiced for restoration of motor functions, a skeletal muscle cell may be used. Exemplary cell types include, but at not limited to, photoreceptor cells (e.g., rod cells or cone cells), bipolar cells, retinal ganglion cells (RGCs), corneal epithelial cells, and Müller cells. In some cases, the method is practiced for fusion protein expression of human optic neuronal cells located within a human eye.

The light used for activation of the engineered fusion protein of this invention, which comprises an optic-responsive element and a downstream signaling element, generally has a wavelength within the range of 440 nm to about 500 nm, e.g., about 480 nm. Depending on the location of the target host cell expressing the fusion protein, the activating light signal may be set at an intensity of 10-100 μW/cm², 20-50 μW/cm², 100-1000 μW/cm², or 200-600 μW/cm², or about 400 μW/cm², and the length of time for blue light exposure ranges from 1-24 hours, 2-12 hours, 1-5 days, 1-6 weeks, or 2-4 weeks, or about 3 weeks, with the lower end of intensity and time duration for in vitro studies and the higher intensity and longer duration for in vivo studies or treatment applications.

As described above, the present invention supports a 1-fusion protein light-activated signaling system as well as a 2-protein light-activated signaling system. In the 1-protein system, only the fusion protein comprising an optic-responsive element and a downstream signaling element is expressed in a target host cell. Upon activation by light exposure at an adequate intensity for an adequate length of time, the fusion protein is able to activated the relevant downstream signaling pathway and the relevant downstream cellular events, ultimately leading to the desired outcome of target cell proliferation or regeneration etc. In some cases, the fusion protein further includes a cell membrane-targeting element (e.g., an N-terminal Lyn tag or a CAAX motif), such that the fusion protein will be localized at or near the host cell membrane in order to optimize the fusion's responsiveness to the light signal.

In the 2-protein system, two fusion proteins are expressed concurrently in the same target host cell: the first is a fusion protein comprising an optic-responsive element and a downstream signaling element, which is the main protein responsible for conducting signal transduction upon light exposure. An exemplary optic-responsive element is derived from the wild-type CRY2 protein, including its mutated or truncated form as described above and herein. Typically in this scheme and in contrast to the 1-protein scheme, this first fusion protein does not include any cell membrane-targeting element, as the purpose of cell membrane localization is achieved via the presence of a second fusion protein, which comprises a cell membrane-targeting element and a CRY2-binding element, thus capable of binding to the first fusion protein via its CRY2-derived optic-responsive element and bringing the first fusion protein to the host cell membrane. An exemplary cell membrane-targeting element used in the second fusion protein is a CAAX motif, and an exemplary CRY2-binding element used in the second fusion protein is CIBN, an N-terminal segment of the CIB1 protein in a length of less than 200 amino acids (e.g., 1-170 segment of SEQ ID NO: 4).

In either system, the first or second fusion protein optionally may further include a detectable element, for example, an mCherry or a GFP element, that allows determination of the presence and/or quantity of the fusion protein in a host cell.

When using either the 1-protein or 2-protein system for in vitro or in vivo studies or for therapeutic applications, a nucleic acid comprising a polynucleotide sequence encoding the first fusion protein, optionally also a polynucleotide sequence encoding the second fusion protein, is first introduced into the target host cell a step of introducing into the cell for the expression of one or two fusion proteins. As disclosed herein, expression vectors such as viral vectors (e.g., adenovirus vectors or adenovirus-associated (AAV) vectors) are often used for their excellent transfection efficiency. In some cases, the first and second fusion proteins are encoded by and therefore introduced into the target host cell by way of a single vector (e.g., one single AAV vector), whereas in other cases, the first and second fusion proteins are encoded by and introduced into the cell by way of two separate viral vectors (e.g., two separate AAV vectors). Typically, direct delivery of the viral vector(s) to the target cells is preferred, for example, intravitreal injection is an effective means for delivering viral vector(s) to RGCs located in a human eye.

This invention further provides a kit for activating a signal transduction pathway in a cell through delivering an optic signal to the cell. The kit comprises at least one container containing a composition comprising a nucleic acid construct encoding the first fusion protein described above and herein, i.e., one that comprises an optic-responsive element derived from a protein naturally responsive to light (e.g., a mutant or truncated form of CRY2 protein) and a downstream signaling element derived from a naturally occurring signal transduction protein (e.g., the intracellular domain of a receptor-type protein such as EGFR, FGFR, or TrkA/B/C, or the signaling domain responsible for mediating or passing signals further downstream, taken from a signaling molecule such as AKT or SOS protein). In some cases, the kit also comprises a second container containing a second composition comprising a second nucleic acid construct encoding the second fusion protein described above and herein, i.e., one that comprises a cell membrane-targeting element and a CRY2-binding element. Typically in such case where there are two containers in the kit, the first fusion protein does not contain any cell membrane-targeting element. In some embodiments, each of the nucleic acid constructs is an expression vector, such as a viral vector like AAV vector. Optionally, the kit further includes users' instructions for how to effectively transfect target cells, e.g., mammalian cells, especially human cells such as human retinal ganglion cells (RGCs), with the nucleic acid construct(s).

### EXAMPLES

The following examples are provided by way of illustration only and not by way of limitation. Those of skill in the art will readily recognize a variety of non-critical parameters that could be changed or modified to yield essentially the same or similar results.

### Example 1 Tunable Axon Regeneration and Neuroprotection of Retinal Ganglion Cells for Vision Preservation by Optogenetic TrkB Activation

Retinal ganglion cells (RGCs) transmit visual information to the brain via their axons in the optic nerve. With RGC damage causing irreversible vision loss due to its non-regenerative nature, strategies safeguarding RGCs and regenerating axons have been extensively explored and promising therapeutic targets identified, including BDNF/TrkB pathway, to combat ocular diseases like glaucoma. However, effective and customizable treatments are still lacking to match patient-specific and temporally evolving damage severities. Here we employed the optogenetic strategy Opto-TrkB to manipulate TrkB signaling with light. In mice with optic nerve injury, mild blue light illuminated in the cage effectively elicited TrkB signaling in RGCs, vigorously promoting RGC survival and axonal regeneration. Such intervention could be finely tuned, as longer or higher-intensity illumination empowered more effects. Turning on Opto-TrkB for 4 weeks regenerated long-distance axons. Switching it on before injury facilitated post-injury axon regrowth. Moreover, after excitotoxic injuries to RGC soma, Opto-TrkB activation supported RGC survival, protected RGC brain projection, and preserved visual functions. Our results present Opto-TrkB as a robust, switchable, and tunable strategy to repair RGCs.

### INTRODUCTION

Retinal ganglion cells (RGCs), the sole output neurons of the eye, transmit visual signals to the brain through the optic nerve which is formed by RGC axons. RGC deaths or axon injuries lead to vision impairment and blindness, the final pathway in many debilitating ocular diseases such as retinal ischemia, diabetic retinopathy, and glaucoma¹⁻⁴. Given the inability of adult mammalian central nervous system (CNS) neurons to spontaneously regenerate, strategies protecting RGCs and regenerating RGC axons hold great promise to halt the progression or even cure those eye diseases. While significant strides have been made in understanding CNS regenerative failures and developing treatments to promote RGC survival and axon regeneration⁵⁻⁸, a persistent challenge remains unresolved. There is a pressing need for therapies that are not only non-invasive and effective but also tunable and dosage-adjustable, offering the flexibility and controllability to be tailored to the variable and dynamically evolving severity of RGC damage in patients.

Brain-derived neurotrophic factor (BDNF) has been long identified as a potent neuroprotective and regenerative factor for RGCs, preventing or slowing RGC death and enhancing axon regeneration in different animal models of eye diseases⁹⁻¹⁶. Thus, various approaches have been explored to supplement BDNF for RGCs, including topical administration, intravitreal injection, subretinal injection, and artificial graft transplantation with BDNF¹⁶⁻²⁰. BDNF preferentially binds with the tropomyosin receptor kinase TrkB to trigger downstream signaling cascades²¹⁻²⁴. Mimetic ligands activating TrkB receptors have also been investigated²⁵. In addition, genetic approaches enabling long-term transgene expression of BDNF and/or TrkB have been tried to avoid recurring in vivo protein delivery^{11,12,26}. Despite a large body of promising preclinical results and tremendous efforts, clinical translation of such ligand-based methods has fallen short due to nonspecific activation by drug diffusion, insufficient TrkB expression, and receptor downregulation induced by sustained stimulation²⁷⁻²⁹. Additionally, BDNF may also activate p75, which can elicit apoptotic signalings^{22,30-32}. Uncontrolled and excessive stimulation of TrkB pathway, whether through ligand diffusion or continuous gene expression, poses risks to tumor development³³⁻³⁵. Therefore, specific and controllable activation of TrkB, while obviating the limitations associated with ligand-based approaches, is critical for fully unleashing the neuroprotective and regenerative potential of the pathway.

Here, to tackle both the challenges of achieving customizable treatment and harnessing the therapeutic promise of TrkB pathway, we leveraged the optogenetics-controlled TrkB activation (Opto-TrkB) to safeguard RGC against damage³⁶. Optogenetics combines optic and genetic approaches to achieve optical manipulation of cell activities with non-invasiveness and high-resolution control in time and dose. In our approach, RGCs in mice were genetically modified to express engineered TrkB receptors that can be activated only by blue light rather than ligands, circumventing all the drawbacks of ligand usage. Via easy and highly controllable delivery of LED blue light into mice cages, mild levels of blue light were sufficient to elicit the TrkB downstream signaling in RGCs. In mice with optic nerve injuries, blue light illumination potently promoted RGC survival and axonal regeneration. Capitalizing on the on/off switchability and tunability in activation levels of optogenetics, the extent of neuroprotection and axon regeneration could be precisely customized: stronger effects were enacted via blue light illumination of longer duration or at higher intensity, and vice versa. In particular, turning on Opto-TrkB for 4 weeks regenerated long-distance axons, while switching it on before injury aided post-injury axon regrowth. In addition, in mice receiving N-Methyl-D-aspartic Acid (NMDA)-induced excitotoxic injury of RGC soma, light-triggered TrkB signaling supported RGC survival, protected RGC brain projection, and preserved visual functions. Our results present Opto-TrkB as a powerful, highly controllable protective and regenerative strategy targeting RGCs in varied pathological conditions, underscoring the promise of optogenetics-facilitated treatments for a broad spectrum of precision therapeutic interventions.

### Results

Opto-TrkB Activates TrkB Signaling in RGC, Promotes RGC Axon Regeneration, and Enhances RGC Survival after Optic Nerve Injury

To achieve precise and efficient optical activation of TrkB signaling, we employed the optogenetic strategy Opto-TrkB³⁶. Endogenous TrkB receptor consists of an extracellular domain, a transmembrane domain, and an intracellular domain (iTrkB). BDNF binds to its extracellular domain and causes receptor dimerization, which auto-phosphorylates tyrosine residues in iTrkB and activates the signaling^{37,38} Opto-TrkB hinges on light-gated Arabidopsis cryptochrome 2 (CRY2)-CRY2 and CRY2-CIBN association, both of which are reversible upon blue light removal^{36,39-42}. It contains two components, (1) iTrkB fused with CRY2 and (2) CIBN linked with the cell membrane targeting sequence CAAX. Upon blue light stimulation, CRY2-CIBN association brings iTrkB onto the cell membrane where CRY2-CRY2 binding prompts iTrkB homo-interaction, thus activating TrkB signaling (FIG. 1A). Without the TrkB extracellular domain and the need for ligands, Opto-TrkB is freed from all the limitations associated with ligand-dependent approaches. In PC12 cells expressing Opto-TrkB, blue light effectively elevated phosphorylated ERK (p-ERK) and AKT (p-AKT) levels and promoted neurite growth (FIG. 9A-D). U0126 (MEK inhibitor) or LY294002 (PI3K inhibitor) treatment blocked the increase of p-ERK or p-AKT levels, respectively (FIG. 9B), while U0126 treatment suppressed light-induced neurite growth (FIG. 9C-D), validating the light-gated activation of downstream signalings and neurite growth.

Proceeding to evaluate the neuroprotective and regenerative efficacy of Opto-TrkB in vivo, we first set out to confirm its efficient expression and light-gated signaling activation in RGCs in mice. AAV serotype 2/2 particles of AAV-3HA-CRY2-iTrkB and AAV-CIBN-GFP-CAAX with the hSyn promoter were intravitreally injected into C57BL/6J mice to co-express the two components of Opto-TrkB in RGCs. Over 90% of RGCs expressed both fusion proteins four weeks after AAV injection (FIG. 10). Next, we examined the optical activation of TrkB signaling. We started with the mouse optic nerve crush (ONC) injury model, which has been widely used to study optic nerve injury and assess both the protective and axonal regenerative capabilities of a treatment⁴³. In this work, blue light was remotely delivered from programmable blue light LED arrays installed on the cage walls (FIG. 1B). Mice with RGC expressing GFP were used as a control. ONC was introduced 4 weeks after AAV injection. One day after dark adaptation, mice were placed in the dark or illuminated with 600 μW/cm² blue light for 2 hours before being sacrificed (FIG. 1C). Blue light exposure resulted in increased p-ERK and p-AKT levels in RGCs expressing Opto-TrkB compared with the dark or GFP groups, validating effective light-inducible TrkB signaling in RGCs in vivo (FIG. 1D-G).

Following that, we embarked on evaluating the efficacy of Opto-TrkB promoting RGC survival and axon regeneration after axonal injuries. Four weeks post-AAV injections to express GFP or Opto-TrkB, mice underwent ONC. One GFP group and one Opto-TrkB group were exposed to 600 μW/cm² blue light for 2 weeks, with parallel control groups for each that were kept in the dark (FIG. 1H). Then RGC axons were labeled by intravitreal injection of FITC-conjugated Cholera Toxin Subunit B (CTB-FITC), and Tuj1 staining marked surviving RGCs in the retina. The GFP groups, either maintained in the dark or illuminated with blue light, exhibited little axon regeneration and no significant difference in RGC survival. In mice expressing Opto-TrkB, the dark group showed little change in RGC survival but slightly improved axon regeneration, possibly due to Opto-TrkB's basal activities. In stark contrast, blue light exposure significantly enhanced RGC survival and stimulated robust axon regeneration, with many regenerated axons traversing the lesion site and some extending up to 2 mm beyond it (FIG. 1I-L).

We also conducted a side-by-side comparison between Opto-TrkB and ciliary neurotrophic factor (CNTF), a highly potent neuroprotective and regenerative cytokine^{44,45} which has been utilized in clinical trials for RGC neuroprotection in glaucoma patients^{46,47} Here, CNTF was supplemented by AAV-mediated expression, effectively triggering the downstream STAT3 signaling (Fig S3A-B). Both CNTF gene overexpression and Opto-TrkB activation significantly boosted axon regeneration, with CNTF slightly outperforming Opto-TrkB (FIG. 11C-D). Conversely, Opto-TrkB activation promoted more RGC survival than CNTF gene overexpression (FIG. 11E-F). Our results demonstrate the potent neuroprotective and regenerative capability of Opto-TrkB.

### Opto-TrkB Induces a Multimodal Network of Signaling Events to Enhance RGC Axon Regeneration

To delineate the molecular events underlying Opto-TrkB-enacted survival and axon regeneration of RGC across time, bulk RNA-sequencing (RNAseq) was performed using RGCs harvested from 4 different experimental conditions, mice expressing GFP or Opto-TrkB at 3- or 14-days post injury (denoted as GFP 3d, Opto-TrkB 3d, GFP 14d, and Opto-TrkB 14d, respectively). To collect RGCs for 3d groups, microruby, a fluorescent neuronal tracer, was injected at crush sites immediately following ONC to retrogradely label the injured RGCs (FIG. 2A). In contrast, for 14d groups, microruby injections were administered 12 days after injury to different locations: around the lesion site in GFP mice to label surviving RGCs, and 1.5 mm distal to the lesion in Opto-TrkB mice to selectively label surviving RGCs with regenerated axons >1.5 mm (FIG. 2A). At designated time points, microruby-labeled RGCs were manually and randomly selected from dissociated retinas for RNAseq analysis (FIG. 2B).

Among the 3d and 14d groups, different numbers of differentially expressed genes (DEGs) were identified through comparisons between RGCs harvested from mice expressing Opto-TrkB and GFP (3d: DEG_{UP}=470 and DEG_{DOWN}=28; 14d: DEG_{UP}=489 and DEG_{DOWN}=32) (FIG. 2C). The majority of DEGs identified in 3d groups were different from 14d groups (Noncommon: 3d: 91.6% DEG_{UP} and 92.3% DEG_{DOWN}; 14d: 91.9% DEG_{UP} and 93.5% DEG_{DOWN}), implying that Opto-TrkB elicited distinct molecular events at different time points (FIG. 2D). Nevertheless, a minute number of DEGs (36 genes) were both identified in 3d and 14d (FIG. 2D, Venn diagram), and most of them were implicated in the regulation of axonogenesis (FIG. 2D, box plots), thus confirming that axonal regeneration is one of the common events triggered by Opto-TrkB over time. To unbiasedly investigate system-level activities of RGCs evoked by Opto-TrkB, functional over-representation analysis (ORA) of all DEGs identified at both 3d and 14d was conducted⁴⁸. The analysis revealed that Opto-TrkB initiated pathways facilitating neural repair [i.e., “positive regulation of axonogenesis”; “axonal transport of mitochondria”, and “positive regulation of autophagy”] as well as intracellular signalings supporting such cellular processes [i.e., positive regulation of type I interferon production⁴⁹; IL2 signaling⁵⁰⁻⁵²; protein glycosylation^{53,54}, TNFR1-induced NF-kappa-B signaling pathway^{55,56}] (FIG. 2E). A closer examination uncovered that although genes associated with autophagy, axogenesis, type I interferon signaling, and IL2 signaling were upregulated in both 3d and 14d Opto-TrkB groups, DEGs enriched in 14d Opto-TrkB group were substantially different and more pronounced compared with those in 3d Opto-TrkB group, indicating a dynamic series of intracellular events might have occurred at different stages of Opto-TrkB-enabled neural repair (FIG. 2F). To further probe the temporally evolving events, we conducted time course-specific gene set enrichment analysis (GSEA) against annotated pathways from the Reactome database that annotates mainly molecular signaling and regulatory pathways⁵⁷. At 3d, Opto-TrkB activation triggered the ERK/MAPK pathway (FIG. 2G, top), which may serve as an early signal upon axon injury to mediate survival and axon regrowth⁵⁸. At 14d, a cytokine-related signaling network [i.e., interferon signaling⁴⁹, interleukin and granulocyte-macrophage colony-stimulating factor (GM-CSF) signaling^{59,60}] that accelerates nerve regeneration was activated instead (FIG. 2G, bottom). Together, these findings illustrate that Opto-TrkB activation can effectively induce a network of signaling events that support the axon regeneration of injured RGCs.

Opto-TrkB Temporal Control Reveals that Sustained Activation Preserves More RGCs and Promotes Long-Distance Axon Regeneration

Leveraging the switchability of Opto-TrkB activation, we probed how different durations of TrkB signaling influenced the effect. Such temporal control relies on the reversible nature of CRY2-CRY2 and CRY2-CIBN interactions, allowing for the cessation of signaling activation by simply turning off blue light at any desired time point. Four weeks after the AAV injection, mice expressing Opto-TrkB underwent ONC injuries and were then randomly divided into four groups. Over the following two weeks, three groups received 2, 5, or 8 days of blue light exposure, respectively, followed by darkness, while a fourth group was exposed to blue light for 14 days (FIG. 3A). Then tissue analysis was performed to evaluate the resulting neuroprotection and axon regeneration. While a brief 2-day Opto-TrkB activation was sufficient to promote significant RGC axonal regeneration and survival compared to the GFP group, prolonged stimulation elicited more and longer regenerated axons as well as more RGC survival (FIG. 3B-F). Interestingly, 14 days of stimulation significantly enhanced RGC survival than 2 days (FIG. 3E-F), indicating that transient TrkB activation is insufficient for certain populations of RGCs to combat the persistent stress from axotomy.

Then we further extended blue light treatment to 4 weeks to examine the potential for long-distance axon regeneration. Regeneration of lengthy axons is essential for reconnecting injured areas with distant targets, representing a critical clinical goal that, however, remains a long-standing challenge. In the same cohort of mice, one eye in each mouse expressed GFP as a control, and the other eye expressed Opto-TrkB. Both eyes were subjected to blue light illumination (600 μW/cm²) for four weeks (FIG. 4A). The eyes expressing GFP demonstrated minimal axon regeneration, and the number of surviving RGCs was only half that of the Opto-TrkB group (FIG. 12). In contrast, prolonged activation of Opto-TrkB resulted in significant and extensive axon regeneration, with some axons even reaching the optic chiasm (FIG. 4B-C). Taken together, Opto-TrkB enables neuroprotection for customizable durations and allows long-distance axonal regenerations by setting sustained activation.

### Switching on Opto-TrkB Signaling Prior to Injury Promotes Post-Injury Axon Regeneration

In light of the protection provided by pre-damage BDNF/TrkB signaling activation shown previously⁶¹, we investigated the effects of turning on Opto-TrkB before the axonal injury, utilizing the versatile switchability of Opto-TrkB. To this end, two weeks after AAV-Opto-TrkB injection, Opto-TrkB was switched on in one group for 2 weeks before ONC induction, while another group did not receive Opto-TrkB activation as a control. Following ONC, both groups were maintained in the dark for another 14 days prior to tissue analysis (FIG. 5A), ensuring that any observed effects could be specifically attributed to the pre-injury Opto-TrkB activation. Compared with the GFP control group that consistently showed minimal axonal regeneration and RGC survival (FIG. 5B, E), Opto-TrkB dark group showed slightly enhanced axonal regeneration due to basal activities of Opto-TrkB. Conversely, pre-injury Opto-TrkB activation empowered a notable improvement in both the number and the length of regenerated RGC axons (FIG. 5C-D). Intriguingly, the amounts of surviving RGCs remained similar among all the groups (FIG. 5E-F), indicating that pre-injury TrkB activation was not sufficient to protect RGCs from death. It is worth noting that the enhancement of axon regeneration offered by the 2-week pre-injury Opto-TrkB activation was much weaker than a 2-week post-injury Opto-TrkB activation shown in FIG. 1 and FIG. 3. Indeed, we found that pairing pre-injury Opto-TrkB activation (ranging from 9 to 28 days) with a subsequent 2-week post-injury activation failed to further enhance not only RGC survival but also axonal regeneration compared to post-injury Opto-TrkB activation alone (FIG. 13). These results confirmed that although pre-injury Opto-TrkB activation itself could aid axon regeneration to a certain degree, it was considerably less potent than post-injury Opto-TrkB activation, thereby exerting negligible additive reparative effects when combined with post-injury TrkB activation.

### Adjusting Opto-TrkB Activation Strength Tunes the Level of Neuroprotection and Axon Regeneration

The capacity to tailor the dosage as needed is vital for precision and personalized treatment. Here exploiting the tunability of Opto-TrkB activation levels by altering light stimulation intensities, we probed the potential for dose modulation in optogenetics-mediated neuroprotection. Right after AAV-Opto-TrkB injection, mice were randomly separated into 4 groups, either kept in the dark or received blue light illumination at intensities increasing from 30 μW/cm², 120 μW/cm², to 600 μW/cm² for 6 weeks. ONC was introduced 4 weeks after AAV injection (FIG. 6A). Compared to the GFP group or Opto-TrkB dark group, mice that received blue light stimulation, including the group at 30 μW/cm², all exhibited significantly enhanced axon regeneration of RGCs (FIG. 6B-D). In particular, higher blue light intensity yielded more and longer regenerated axons. 600 μW/cm² illumination produced the best RGC survival rate, followed by 120 μW/cm². However, weak Opto-TrkB activation, actuated by 30 μW/cm² blue light, did not noticeably promote RGC survival (FIG. 6E-F), echoing the finding in FIG. 5 that pre-injury Opto-TrkB activation supported RGC axon regeneration rather than survival. These results indicate that a threshold of TrkB activation may be required to promote RGC survival. The elucidation of the underlying mechanisms awaits further investigations.

Opto-TrkB Activation Protects RGC from Excitotoxic Injury Induced by NMDA

To comprehensively evaluate the neuroprotective capability of Opto-TrkB across different types of injuries, we utilized the NMDA-induced excitotoxicity model where RGC somas are damaged^{62,63}, complementary to ONC models that inflict injury on RGC axons. NMDA (5 mM, 2 μl) was injected into the eye 4 weeks after AAV-Opto-TrkB or AAV-GFP injection (FIG. 7A). As an uninjured control, PBS was injected into mice expressing GFP. For the Opto-TrkB group, blue light activation started one day before NMDA injection and lasted for 15 days in total. The majority of RGCs were dead in the injured GFP group (FIG. 7B-C). On the other hand, the injured group with Opto-TrkB activation showed nearly double the number of surviving RGCs (FIG. 7B-C). Previous research has demonstrated the resilience of αRGCs and ipRGCs to NMDA-induced damage^{64,65}. In the injured GFP group, approximately half of the SMI32-positive αRGCs survived. Notably, Opto-TrkB activation further improved the post-injury survival rate of αRGCs (FIG. 14A-B). Conversely, melanopsin-positive ipRGCs, which are inherently more resistant to excitotoxic injury, did not exhibit significant changes in survival rates among different experimental groups (FIG. 14C-D).

Next, we investigated whether RGC axons, with their projections to the brain, were protected by Opto-TrkB activation from NMDA-induced excitotoxicity. To label the RGC axons and their brain projections, Alexa Fluor 555-conjugated CTB (CTB-555) was injected into the vitreous chamber of the eye (FIG. 7D). Similar to RGC somas, RGC axons within the optic nerve severely degenerated in the injured GFP group. However, Opto-TrkB activation significantly preserved RGC axons, as shown by the one-fold enhancement of CTB intensity (FIG. 7E-F). RGC axons predominantly project to the contralateral hemisphere in mice, including the suprachiasmatic nucleus (SCN), the lateral geniculate nucleus (LGN), the olivary pretectal nucleus (OPN), and the superior colliculus (SC). We examined the preservation of RGC axonal projections in these regions. While the SCN, which receives projections from melanopsin-positive ipRGCs, exhibited intact RGC projections (FIG. 7G-H), a significant loss of target projections was observed in the LGN, OPN, and SC regions in the injured GFP group (FIG. 7I-N). However, Opto-TrkB activation effectively conserved axonal projections in the LGN, OPN, and SC regions (FIG. 7I-N). In summary, Opto-TrkB activation not only protects RGC survival but also preserves brain projections from excitotoxic injury induced by NMDA.

Opto-TrkB Activation Preserves the Visual Function after NMDA-Induced Excitotoxic Injury

The ultimate goal of RGC-targeted neural repair is to preserve functional vision. Therefore, we investigated the potential of Opto-TrkB to protect visual functions using the NMDA-induced excitotoxicity model which maintains optic nerve integrity to transmit visual information. Similar to experiments in FIG. 7, mice expressing GFP were injected with PBS as an uninjured control or injected with NMDA as an injured control. NMDA-induced excitotoxicity was initiated in mice expressing Opto-TrkB one day after the blue light illumination was switched on. Two weeks later, multiple visually guided behavioral tests were conducted, including optomotor response, looming stimulus, visual cliff, and pupillary light reflex (PLR).

In the optomotor response assay, mice were placed on a platform at the center of a chamber surrounded by four computer monitors, which displayed rotating sinusoidal gratings of alternating white and black stripes (FIG. 8A). The highest frequency grating that triggered an optomotor response, a reflex turning of the head, from the mouse indicated its visual acuity⁶⁶. Uninjured control mice predominantly exhibited visual acuity between 0.3 to 0.4. However, injured GFP mice exhibited a severe impairment, with most visual acuity below 0.1 (FIG. 8B). Injured mice with Opto-TrkB activation experienced a lesser decline in visual acuity, performing notably better than the injured GFP group, with some mice achieving measures above 0.2 (FIG. 8B).

We then performed a looming stimulus test to evaluate the mouse's visual-based innate defensive behavior^{67,68}. The test took place in a specialized chamber featuring a shelter, an overhead monitor for stimuli display, and a camera to record responses (FIG. 8C). A positive reaction was marked by escape or freezing behavior upon the looming stimulus. In the uninjured group, 23 out of 25 mice responded to the stimulus. However, the injured GFP group had a dramatic decrease in response, with only 2 out of 24 reacting. Notably, Opto-TrkB activation significantly preserved the defensive responses post injury, with 18 out of 23 mice responding (FIG. 8D).

We used a visual cliff test to evaluate depth perception in mice, leveraging their natural aversion to a cliff's deep side^{69,70}. Mice were placed on a platform between an illusory steep drop and a shallow side (FIG. 8E). First, we recorded whether the mouse's initial step from the platform was towards the shallow (shallow side choosers) or deep side (deep side choosers), without considering any prior behavior of surveying the surroundings. More injured Opto-TrkB mice chose the shallow side than injured GFP mice, but without statistical significance (FIG. 8F). However, this measurement may underestimate the difference in visual function, as a blind mouse might randomly select the shallow side. For better accuracy, we tracked if mice examined their surroundings prior to choosing. Avoidance of the deep side after surveying the surroundings (deep side avoider), rather than a random selection without surveying (chooser without survey), indicates depth perception and an effort to prevent falling. 23 out of 26 uninjured mice and only 1 out of 25 injured GFP mice exhibited deep side avoidance behavior. However, with Opto-TrkB activation, 12 out of 24 injured mice showed deep side avoidance behavior (FIG. 8G). Additionally, we recorded the latency to leave the center platform, from placement to hindlimb departure. Uninjured mice typically spent several seconds surveying before choosing, and injured GFP mice rarely paused. However, many injured mice with Opto-TrkB activation did pause to survey before leaving the platform (FIG. 8H).

Lastly, we performed PLR tests to measure pupil constriction in response to light stimulation, a response mediated by RGC axonal projections to the OPN^{71,72}. Mice were first adapted to darkness to fully dilate the pupils. Then, blue light was shone into their eyes to trigger pupil constriction, while an infrared camera recorded the pupil changes. The minimum pupil area after stimulation was compared to the pre-stimulation area for quantitative analysis. Uninjured mice showed a pronounced constriction, over 80%. Injured GFP mice showed impaired pupil constriction, with only 37% constriction. However, injured mice with Opto-TrkB activation exhibited enhanced PLR, with nearly 60% pupil constriction (FIG. 8I-J). Additionally, by measuring the time interval from stimulation onset to peak pupil constriction, no latency differences were found among the groups (FIG. 8K), indicating that the latency was not affected by varying degrees of RGC axon innervation to OPN.

The preservation of visual function by Opto-TrkB was also shown by assessing neuronal activities within RGC projection targeted areas using c-Fos staining. After performing the behavioral tests, mice were dark-adapted for 10 hours, followed by 1-hour room light exposure. CTB-555 was intravitreally injected to indicate the SCN, LGN, OPN, and SC regions. Whereas the numbers of c-Fos-positive cells in LGN, OPN, and SC were significantly reduced by NMDA treatment compared to the uninjured group, Opto-TrkB activation notably enhanced them.

Taken together, our results showed that Opto-TrkB activation is able to preserve the visual function from excitotoxic injury.

### Discussion

In this work, we first presented Opto-TrkB, the optogenetic strategy to control engineered TrkB receptors remotely and precisely, as an effective approach to trigger TrkB signaling in RGCs in vivo and boost both the axon regeneration and survival of RGCs after axonal injuries. Next, we revealed that by tailoring the dosage of TrkB activation, the amount of effects could be fine-tuned: prolonged or stronger TrkB signaling conferred more RGC neuroprotection and axon regeneration. This unmatched controllability relies on the fact that via Opto-TrkB, TrkB signaling can be switched on/off and tuned up/down at any desired time points and to any desired levels by adjusting the light illumination conditions. With this, we unveiled that activation of TrkB prior to axonal injuries enhanced post-injury axon regeneration rather than survival of RGCs. Furthermore, we demonstrated the potent neuroprotection enabled by Opto-TrkB for RGCs with excitotoxic injuries. Not only the number of surviving RGCs and their brain projections were conserved, but also the visual function could be protected.

The eye's transparency and accessibility to light make it an optimal organ for optogenetic interventions. A human clinical trial (NCT02556736) has been launched to investigate ion channel-based optogenetics for restoring vision in retinitis pigmentosa patients⁷³. Besides, because of the closed physical space for local gene delivery and immune privilege of the eye, ocular disorders present attractive targets for gene therapies, as demonstrated by the FDA's approval of LUXTURNA™ in 2017 for treating inherited retinal dystrophies⁷⁴. In our study, light was used as the remote switch, conferring unmatched advantages and controllability over signaling activation in RGCs. Not only can the signaling be switched on or shut down at any desired time point, but also the dosage and strength of signaling can be tuned at any time, enabling customizable and precise treatments that can be conveniently adjusted and accurately designed to meet specific or dynamically evolving needs. Thus, our optogenetic approach provides new possibilities and promising strategies to address the pressing need for controllable and adaptive neuroprotection.

We have shown that light-mediated TrkB signaling via Opto-TrkB yielded robust reparative effects in promoting both axon regeneration and survival of RGCs, which is close to the potency achieved by CNTF stimulation. Our optogenetic method outperforms the conventional methods utilizing BDNF or other TrkB-agonists for several reasons. First, those conventional methods may be limited by insufficient expression of TrkB in some RGCs or the downregulation of TrkB induced by long-term ligand treatment^{16,19,27-29}. However, Opto-TrkB activation obviates the usage of ligands and does not hinge on endogenous TrkB receptors, allowing for efficient TrkB activation in neurons with little or insufficient expression of endogenous TrkB and over prolonged duration. Indeed, compared with two-week Opto-TrkB activation, four-week activation further enhanced optic nerve regeneration, both in length and number, and RGC survival was also maintained at similar levels (FIGS. 4 and 14). Secondly, Opto-TrkB can produce purer neuroprotective effects than ligand-based means because it does not elicit the apoptotic p75 signaling, where p75 is the low-affinity receptor for BDNF^{22,30-32}. Thirdly, only transduced RGCs will be stimulated, not perturbing other cells in the eyes, while ligand-based methods will stimulate all the surrounding cells expressing TrkB. In the fourth place, the signaling activation of Opto-TrkB can be switched off at any desired time point, avoiding over-activation of TrkB signaling and thus the risks of causing cancers. Opto-TrkB can be potentially used to manipulate TrkB signaling in cells other than RGCs in the eye, allowing it to target other ocular disorders such as eye surface diseases and retinal diseases. In addition, it has the potential to be used to treat disorders such as stroke and spinal cord injury when supported by in vivo light delivery technologies such as small wireless LED devices or upconversion nanoparticles⁷⁵⁻⁷⁸. Thus our work sets the stage for a broader spectrum of therapeutic interventions.

Regenerating long-distance axons, a clinically critical step in connecting injured sites to distal targets, may require the sophisticated orchestration of multiple signaling pathways, such as mTOR, ERK, STAT1, and STAT3 signaling^{49,79-81}. However, manipulating several signaling pathways precisely and simultaneously remains technically challenging, which calls for the joining of new methods into the orchestra. Distinct from conventional methods that depend on the supplementation of ligands, optogenetics uses light, a completely orthogonal and precisely programmable physical stimulus, to activate targeted signaling pathways. Therefore, integrating optogenetics with other methods can offer another dimension of fine control. Here, we showed that combining Opto-TrkB activation and CNTF supplementation together triggered much more and longer regenerated axons compared to either treatment alone, especially at distances longer than 1 mm (FIG. 11). Such combinatorial approaches can undoubtedly be expanded to optogenetic control of other signaling pathways. Research in the future can further explore orthogonal manipulations of different triggers and the mixing of different strategies/signaling pathways to fully harness the power of optogenetics in long-distance axon regeneration.

We found that weak activation of Opto-TrkB did not significantly promote RGC survival, but it could facilitate mild RGC axon regeneration. This trend was reflected in several experimental conditions in our work, for example, upon weak Opto-TrkB activation either resulting from basal activities of Opto-TrkB in the dark or from activation induced by blue light of low intensity. Intriguingly, light-mediated 14-day Opto-TrkB activation prior to the injury produced similar results, indicating that some residual signaling events persisted after the stop of pre-injury TrkB activation and the induction of injury. While the underlying mechanisms remained to be examined in future work, these findings showcased the capacity of our optogenetic approach to unearth elusive aspects of how cells compute the information encoded by different temporal profiles and strengths of signaling for distinct cellular outcomes.

It is worth noting that the amount of blue light needed for Opto-TrkB activation is minimal and well within the safe range. While 600 μW/cm² blue light was used in most experiments, 30 μW/cm² blue light delivered to the cage was sufficient to activate Opto-TrkB for noticeable axonal regeneration (FIG. 6). In contrast, blue light (400-500 nm) from normal indoor room lighting is >100 μW/cm² and from computer monitors can be >1000 μW/cm², not to mention that blue light from sunlight can be up to 14500 μW/cm^{2 82}. Moreover, since mice were freely moving in the cage and would not directly look at the blue light source on the walls all the time, the actual amount of blue light received by the eyes would be much lower than the intensity measured in the cage. Nevertheless, in our future work, varied versions of Opto-TrkB that possess different light sensitivities and thus require different levels of light stimulation can be developed. We can envision that a specific version can be chosen for optimal neuroprotection according to the lighting conditions in one's living environment.

In conclusion, we utilized an optogenetic approach to manipulate TrkB signaling for robust axon regeneration and neuroprotection of RGCs. Using our method, TrkB activation can be turned on and off, or tuned up or down at any given time point. Leveraging these unique advantages, we demonstrated the tunability of axon regeneration and neuroprotection of RGCs in mice ONC models. In addition, Opto-TrkB protected RGCs from excitotoxic injury and preserved visual functions. We believe that our results showcase the promise of adjustable, customizable, and safe treatment, enabled by optogenetic strategies. This work also opens up new possibilities that such optogenetic approaches can be expanded to other types of cells and diseases as well as other signaling pathways.

### Method Details

### Cloning

The details of CRY2-iTrkB and CIBN-GFP-CAAX can be found in our previous work³⁶. The intracellular domain of TrkB (a.a. 455-822, iTrkB), the photolyase homology (PHR) domain (a.a. 1-498) of Arabidopsis thaliana CRY2, and the truncated version of Arabidopsis thaliana CIB1 (a.a. 1-170) were used. For in vitro experiments in the PC12 cell line, CRY2-iTrkB and CIBN-GFP-CAAX were inserted together with a P2A self-cleaving peptide sequence into a lentiviral vector (Addgene #11795), forming CRY2-iTrkB-P2A-CIBN-GFP-CAAX. As a control, the iTrkB gene was replaced by the mCherry (mCh) sequence to examine the effect of the optogenetic system and blue light, producing CRY2-mCh-P2A-CIBN-GFP-CAAX. For in vivo experiments, Opto-TrkB constructs were separately inserted into an AAV vector (Addgene #74513), generating AAV-3HA-CRY2-iTrkB and AAV-CIBN-GFP-CAAX.

### Cell Culture

PC12 cells (ATCC CRL-1721) were cultured in F-12K medium supplemented with 15% horse serum (HS) and 2.5% fetal bovine serum (FBS). HEK293T cells (ATCC CRL-3216) were cultured in DMEM supplemented with 10% fetal bovine serum. For PC12 neurite growth assay and Western blotting, PC12 cells were transduced via lentivirus and allowed to recover in a complete growth medium for another day. PC12 cells were starved in serum-reduced F-12K medium (with 1.5% HS and 0.25% FBS) overnight before being subjected to blue light stimulation. All mediums were purchased from Thermo Fisher Scientific.

### Lentiviral Transduction

Opto-TrkB or control constructs were inserted into a lentiviral transfer vector and were transfected into HEK293T cells together with pMD2.G and psPAX2 at 2.5:1:2 molar ratio. Media were refreshed 18 hr post transfection. Lentivirus-containing media were harvested 48 hr and 72 hr post transfection, and filtered through a 0.45 μm PES filter before being stored at −80° C. for future use.

### Western Blotting

Cells were pre-treated by 10 μM MEK inhibitor U0126 (MedChemExpress #HY-12031) and/or 20 μM PI3K inhibitor (MedChemExpress #HY-10108) for one hour before light stimulation, followed by 1 mW/cm² blue light irradiation for 10 min at 5 sec on/5 sec off intervals. After treatment, cells were rinsed with cold PBS once and lysed in lysis buffer (Beyotime P0013, supplemented with protease and phosphatase inhibitor cocktail Beyotime P1045) on ice for 15 min. Cell lysates were transferred to a clean 1.5 mL microcentrifuge tube and spined down at 12,000 g, 4° C. for 15 mins. The protein concentrations of supernatants were determined by BCA kit (Beyotime P0010). Equal amount of protein samples was mixed with 5×SDS-PAGE sample loading buffer (Beyotime P0015) and boiled at 95° C. for 5 mins.

SDS-PAGE gels were prepared from the 10% SDS-PAGE Gel SuperQuick Preparation Kit (Beyotime P0690). Approximately 10-20 ug protein was loaded to each lane, and gel electrophoresis was running at 90 V for around 120 mins. The protein was then transferred to a Immobilon®-P PVDF Membrane (Merck Millipore) within a Bio-rad Trans-Blot Turbo Transfer System. The blots were blocked by a 5% milk TBS-T buffer at room temperature for 1 hr. Primary antibodies were used following manufacturer's instructions: ERK1/2 (1:1000, Cell Signaling #9102), phospho-ERK1/2 (1:1000, Cell Signaling #9101), Akt (1:1000, Cell Signaling #9272), phospho-Akt (Ser473) (1:1000, Cell Signaling #4060), GAPDH (1:2500, Cell Signaling #5174).

### Animals

C57BL/6J mice were obtained from Charles River. 6-8 weeks old C57BL/6J mice of both genders were used in this study. All experiments were conducted in compliance with the guidelines of the Laboratory Animal Facility at the Hong Kong University of Science and Technology.

### AAV Packaging

AAV serotype 2/2 with the hSyn promoter was utilized to overexpress GFP and Opto-TrkB genes (AAV-3HA-CRY2-iTrkB and AAV-CIBN-GFP-CAAX). AAV serotype 2/1 with the CMV promoter was used to overexpress CNTF in the retina. The virus titer was determined by qPCR and adjusted to 10¹² GC/mL for all the experiments.

### AAV Intravitreal Injection and Optic Nerve Injury

The mice were given a mixture of ketamine (80 mg/kg) and xylazine (10 mg/kg) to induce anesthesia. The conjunctiva was exposed by clamping the eyelid with an artery clamp. Two microliters of the vitreous body were withdrawn using a Hamilton syringe, followed by the gentle injection of two microliters of the virus into the eye. Eye ointment was applied to the cornea for eye protection, and postoperative analgesia was provided through the administration of ketoprofen (5 mg/kg).

Four weeks after the AAV injection, intraorbital optic nerve crush was conducted using previously described methods⁴³. The mice were anesthetized with a ketamine (80 mg/kg) and xylazine (10 mg/kg) cocktail, and an incision was made on the conjunctiva. The optic nerve was exposed and crushed using jeweler's forceps (Dumont #5; Fine Science Tools) for 2 seconds at a distance of 1-2 mm behind the optic disk. Eye ointment and ketoprofen (5 mg/kg) were applied after surgery.

### Blue Light Stimulation and Light Intensity Measurement

The mice that required blue light stimulation were housed in an individually ventilated cage (IVC). Four blue LED arrays were installed outside the walls of the case. The LED arrays were controlled by a DC power supply with a repeating pattern of 10 seconds ON and 20 seconds OFF.

The optical power meter (THORLABS, PM100D) was used to measure the intensity of light at a wavelength of 480 nm in the mouse cage. The photodiode power sensor of the power meter was positioned at the center point of the cage bottom, facing upward.

### Immunohistochemistry

The sections or whole-mount samples were incubated with the appropriate antibodies as required for the experiment. Primary antibodies: Tuj1 (1:1000, BioLegend, 801202 and 802001), p-ERK (1:500, Cell Signaling, 4370), p-AKT (1:500, Cell Signaling, 3787), FITC (1:500, Invitrogen, 71-1900), HA-Tag (1:500, Cell Signaling, 2367), GFP (1:500, Invitrogen, A10262), SMI32 (1:1000, BioLegend, 801701), Melanopsin (1:500, Advanced Targeting System, AB-N39), c-FOS (1:500, Cell Signaling, 2250), p-STAT3 (1:500, Cell Signaling, 9145). Secondary antibodies: Goat anti-Mouse 555 (1:500, Invitrogen, A-21424), Goat anti-Mouse Cy5 (1:500, Invitrogen, A-10524), Goat anti-Rabbit 555 (1:500, Invitrogen, A-21429), Goat anti-Rabbit Cy5 (1:500, Invitrogen, A-10523), Goat anti-Chicken 488 (1:500, Invitrogen, A-11039).

To determine the number of surviving RGCs under various treatment conditions, whole-mount Tuj1 staining was performed. The mice were given a lethal dose of ketamine/xylazine and perfused with PBS followed by 4% PFA. Eyes were collected and postfixed in 4% PFA overnight before the retinas were dissected and washed with 1×PBS three times. The retinas were then incubated with Tuj1 antibody overnight at room temperature after being blocked with 0.1% Triton X-100 in 4% normal goat serum (NGS, Invitrogen, 50062Z) for 30 minutes. Following incubation with the Tuj1 antibody, the retinas were washed with PBS three times and incubated with a secondary antibody for 1 hour. The tissues were then washed with PBS and mounted onto glass slides for imaging.

For immunostaining of tissue sections, retinas, optic nerves, or brains were dissected and postfixed in 4% PFA overnight in the 4° C. refrigerator. Tissues were then cryoprotected in 30% sucrose overnight and embedded in OCT compound (SAKURA, 4583) at −80° C. Samples were sectioned at −20° C., with 25 μm for the retina and brain and 8 μm for the optic nerve. Tissue sections were then incubated with primary antibodies overnight after being blocked with 0.1% Triton X-100 in 4% NGS for 30 minutes. After the samples were washed three times with PBS, the corresponding secondary antibodies were applied for 1 hour. The samples were then washed with PBS and mounted on coverslips for imaging.

### Quantification

Whole-mount Tuj1 staining was performed to determine the surviving RGCs. After staining, the mounted retinas were imaged by a confocal microscope (Zeiss, LSM 880). For each retina, 12 images were taken from different quarters, covering both the peripheral and central regions of the retina. The number of Tuj1+RGCs was counted by an individual who was blind to the different treatment groups.

To quantify the number of regenerated axons after optic nerve crush, CTB-FITC (Sigma-Aldrich, C1655) was intravitreally injected two days before sacrificing the mice. The collected optic nerves were cryosectioned longitudinally (section thickness: t=8 μm) and stained with the FITC antibody. The tissues were then imaged under a confocal microscope, with five images taken for each optic nerve. To quantify the number of regenerated axons at different distances from the lesion site, the following formula was used: axon number=π²π(n/d)/t. The r is the radius of the optic nerve. The n is the average axon number of the five sections. The d is the nerve width at the counting site. The t is the section thickness (8 μm).

### Microruby Labeling

To label target cells, we performed retrograde labeling of RGCs by gently injecting microruby (500 nL, 5% wt/vol, Invitrogen) into the optic nerve⁴⁹. For the 3-day injury groups, microruby was injected into the lesion site in both the GFP- or Opto-TrkB-expressed mice immediately after ONC. The retina was collected and dissected 3 days later for further processing. For the 14 days injury groups, microruby was gently injected around the lesion site in the GFP-expressed group or around 1.5 mm distal to the lesion site in the Opto-TrkB-expressed group at 12 days post injury. Two days later, the retina was collected and dissected for further processing.

### RGC Isolation and RNA Sequencing

The isolation of RGCs and subsequent RNA sequencing were conducted following previously established protocols⁴⁹. In brief, the RGC-labeled retina was quickly dissected from mice after sacrifice. The retina was then incubated with 0.5 mg/ml papain at 37° C. for 30 minutes. After a single wash with Neurobasal-A medium, the retina was triturated into a single-cell suspension using pipetting. The retinal cells were centrifuged at 4° C. at a speed of 300 g for 6 minutes, followed by resuspension in Neurobasal-A medium and 10% B27 supplement. Manual picking of single microruby-labeled RGCs was performed using mouth-pipetting^{83,84}. A small volume of RGC suspension was transferred into a series of 1% BSA-containing PBS drops until a single microruby-labeled RGC was isolated. Two RGCs were then transferred into one PCR tube containing 4.2 μl of lysis buffer, as specified in the Smart-seq2 protocol⁸⁵, and stored at −80° C. for further processing. Each treatment group consisted of 3 or 4 biological samples, with each sample comprising a diluted mixture obtained from 10 cells, using a dilution ratio of 1/5.

The RGC lysate was further processed following the Smart-seq2 protocol. RNA was reverse transcribed into cDNA using Superscript II and TSO primer, followed by amplification with KAPA HiFi HotStart ReadyMix and ISPCR primer for 26 cycles. Purification of the amplified product was performed using Ampure XP beads. cDNA quality was assessed using a fragment analyzer, while the concentration was determined using the Qubit dsDNA high-sensitivity (HS) kit. qPCR was conducted to measure the relative expression levels of the Sncg and Rbpms genes. Samples with low cDNA quality or low expression of both Sncg and Rbpms genes were excluded from downstream library construction.

Library construction was carried out using the TruePrep DNA Library Prep Kit V2 for Illumina (vazyme) and the TruePrep Index Kit V2 for Illumina, following the manufacturer's protocol. The library was then sent to Novogene for sequencing using the NovaSeq PE150 platform.

### Bioinformatics Analysis of the Bulk RNA-Seq Data

Bulk RNA-sequencing service was purchased and conducted by the Novogene company, and raw data was analyzed by our in-house bioinformatician. Prior to any analysis, the quality of sequencing reads was quality-checked by fastqc. High-quality reads were then mapped to the mouse reference genome (GRCm39) using STAR⁸⁶ with two-pass mode. RSeQC⁸⁷ was used to validate the strand-specific information. FeatureCounts⁸⁸ was used to count the reads mapped to genes by setting the strand specificity parameter as “unstranded”. Differential gene expression analyses between Opto-TrkB and GFP were performed with DESeq²⁸⁹ (v 1.30.1) with the default settings. Genes with adjusted p-value smaller than 0.01 and fold change greater than 2 were considered as significantly changed genes. Over-representation analysis was conducted by the Metascape⁴⁸ with default settings, and subsequent enrichment of results was re-organized using the Cytoscape⁹⁰. Gene set enrichment analysis (GSEA) was performed using GSEA software⁹¹, and the reference target pathways were downloaded from the Reactome database⁹².

### NMDA Treatment

Adult C57BL/6J mice were randomly separated into three groups and received AAV injections in both eyes. Two groups received an AAV-GFP injection for both eyes, while one group received an AAV-Opto-TrkB injection for both eyes. The mice were reared under normal circadian conditions of a daily dark-light cycle before further treatment. After 27 days, the group with double-eye AAV-Opto-TrkB injection received LED blue light stimulation for one day and then received double-eye 5 mM NMDA (N-Methyl-D-aspartic Acid, Sigma-Aldrich, M3262) injection. Meanwhile, one AAV-GFP group received PBS intravitreal injection as an uninjured control, and another AAV-GFP group received a 5 mM NMDA intravitreal injection. After injection, the Opto-TrkB group was continuously reared under blue light stimulation, while the two GFP groups were reared under normal circadian conditions. Twelve days later, mice from all three groups received CTB555 (Alexa Fluor™ 555 Conjugate, Invitrogen, C34776) intravitreal injection to trace the RGC axon projections. Two days later, the mice were terminated for further analysis. To quantify the CTB intensity in the optic nerve and RGC axonal projections in the brain, samples labeled with CTB555 from the three groups were imaged under a confocal microscope. The images were then processed and analyzed using ImageJ software. The CTB intensity was normalized by setting the intensity in the uninjured group to 100, and the intensities in the other groups were adjusted accordingly using GraphPad Prism 9 software. All visual behavior tests were conducted between 14-18 days post-NMDA/PBS treatment. The c-fos staining experiment was conducted 18 days after NMDA/PBS treatment. The mice received CTB555 intravitreal injection 2 days before termination and were dark-adapted for 10 hours before being exposed to room light for 1 hour prior to termination.

### Optomotor Response Test

The optomotor response tracking system (CerebralMechanics Inc. OptoMotry HD System) was used to measure the visual acuity of the mouse⁶⁶. The test was conducted by an individual who was blind to the treatment. The mouse was placed on a platform in the center of a chamber composed of four computer monitors displaying rotating sinusoidal white and black grating gradients. The grating was settled at a speed of 12 degrees per second and 100% contrast. A camera was placed above the platform to monitor the mouse's movement in real time throughout the experiment. The measurement started with a low spatial frequency (0.05 cycle/degree) after the mouse was placed on the platform and allowed to adapt for several seconds. The grating then rotated either clockwise or anticlockwise while the experimenter monitored the mouse's tracking movement for several seconds. If the mouse showed tracking behavior, the spatial frequency was gradually increased until the mouse did not show any response. The highest spatial frequency grating rotated in either direction that triggered an optomotor response from the mouse indicated the visual acuity of the corresponding eye. Each mouse was tested for approximately 10-15 minutes per session. The mean number of the two eyes represented the visual acuity of the mouse.

### Looming Stimulus Test

The looming visual stimulus response was conducted to evaluate visual function in mice⁶⁷. The test was conducted in a customized opaque chamber (45×45×30 cm) with a shelter on the side, which was 12 cm wide and mounted at a height of 8 cm. A camera was installed at a right angle to capture all areas of the chamber, and a computer monitor was set up on the top of the chamber to display a video of a black disk that expanded from 2° to 50° on a white background. The black disk was held at 2° for 250 ms before expanding to 50° in 250 ms. It then remained at 50° for 500 ms before repeating. Each expanding cycle lasted for 1s, and the test included 20 expanding repeats. Food pellets were placed outside the shelter to encourage the mouse to explore the chamber. The mouse was given ten minutes to acclimate to the chamber before the stimulus onset. A mouse was considered to have a positive response if it exhibited escape or freezing behavior upon stimulus onset.

### Visual Cliff Test

The visual cliff avoidance test was used to assess mice's depth perception and visual discrimination abilities^{69,70}. The test was conducted in a transparent acrylic box measuring 80×80×10 cm and with an opening at the top. The box was divided into two equal halves, with a platform in the center. During the test, half of the box was put on the edge of a trolley with a laminated checkerboard pattern as the shallow side. Half of the box was elevated 90 cm above a checkerboard-patterned floor as the deep side. A video camera was positioned at a suitable angle to capture the entire box and document the test. The mouse was placed on the center platform (3 cm wide). We tracked the mouse's first step off the platform to the shallow or deep side, independent of whether it explored its surroundings. We also tracked whether the mouse chose the shallow side after observing its surroundings on the platform, or if it chose either side without exploration. The mouse's exploratory behavior was detected when it probed outwardly and retracted more than twice while keeping its body on the platform. The mouse that chose to avoid the deep side after exploring was called a deep side avoider, meaning that it recognized the visual cliff and tried to avoid falling. However, if the mouse chose the shallow or deep side at random without a survey, it would not display depth perception or visual discrimination skills. In addition, we measured latency, which is the time interval between placing the mouse on the middle platform and the hindlimb leaving it. Each mouse was tested once.

### Pupillary Light Reflex Test

The pupillary light reflex test was conducted according to the established experimental procedure⁷¹ to examine non-image-forming visual function. Before the measurement, the mouse was placed in a dark room for two hours to adapt to the dark environment and to dilate the pupils to the maximum area. Then, the mice were weakly anesthetized and fixed on a mice holder to restrict their movement. The change in the mouse's pupils was recorded using an infrared camera connected to a computer. During the experiment, monochromatic light (480 nm) was used to stimulate the mouse's left eye, while the infrared camera was placed on the right eye and recorded for 60 seconds. After recording, the minimum area of the pupils after light stimulation was measured, and quantitative analysis was conducted by comparing it with the pupil area before stimulation. We also measured the latency for maximum pupil constriction, which is the time interval between the onset of light stimulation and the pupil's maximum contraction.

### Statistical Analyses

Statistical analyses were performed using GraphPad Prism 9 software. Student's t-test was used to compare two independent groups, while ANOVA followed by post-hoc multiple analyses was employed for multiple group comparisons. Fisher's exact test was utilized to compare the injured GFP group and the injured Opto-TrkB activation group in the looming stimulus test and the visual cliff test. The error bars in all statistical analyses represent the standard error of the mean (SEM). ns: not significant, *p≤0.05, **p≤0.01. The symbols ns, * or ** above a line indicates the comparison between the two groups that are linked by the line. If the symbols ns, * or ** are not above a line, they, by default, refer to the comparison between the corresponding group and the first group. This applies to all the statistical comparisons in this paper unless additional explanation is provided.

### REFERENCES

1. Antonetti, D. A., Silva, P. S. & Stitt, A. W. Current understanding of the molecular and cellular pathology of diabetic retinopathy. Nat. Rev. Endocrinol. 17, 195-206 (2021).
2. Weinreb, R. N., Aung, T. & Medeiros, F. A. The pathophysiology and treatment of glaucoma: a review. JAMA 311, 1901-1911 (2014).
3. Schuster, A. K., Erb, C., Hoffmann, E. M., Dietlein, T. & Pfeiffer, N. The Diagnosis and Treatment of Glaucoma. Dtsch. Arztebl. Int. 117, 225-234 (2020).
4. Wang, W. & Lo, A. C. Y. Diabetic Retinopathy: Pathophysiology and Treatments. Int. J. Mol. Sci. 19, (2018).
5. He, Z. & Jin, Y. Intrinsic Control of Axon Regeneration. Neuron 90, 437-451 (2016).
6. Liu, K., Tedeschi, A., Park, K. K. & He, Z. Neuronal intrinsic mechanisms of axon regeneration. Annu. Rev. Neurosci. 34, 131-152 (2011).
7. Boia, R. et al. Neuroprotective Strategies for Retinal Ganglion Cell Degeneration: Current Status and Challenges Ahead. Int. J. Mol. Sci. 21, (2020).
8. Fudalej, E. et al. Neuroprotective Factors of the Retina and Their Role in Promoting Survival of Retinal Ganglion Cells: A Review. Ophthalmic Res. 64, 345-355 (2021).
9. Mey, J. & Thanos, S. Intravitreal injections of neurotrophic factors support the survival of axotomized retinal ganglion cells in adult rats in vivo. Brain Res. 602, 304-317 (1993).
10. Mansour-Robaey, S., Clarke, D. B., Wang, Y. C., Bray, G. M. & Aguayo, A. J. Effects of ocular injury and administration of brain-derived neurotrophic factor on survival and regrowth of axotomized retinal ganglion cells. Proc. Natl. Acad. Sci. U.S.A 91, 1632-1636 (1994).
11. Ren, R., Li, Y., Liu, Z., Liu, K. & He, S. Long-term rescue of rat retinal ganglion cells and visual function by AAV-mediated BDNF expression after acute elevation of intraocular pressure. Invest. Ophthalmol. Vis. Sci. 53, 1003-1011 (2012).
12. Martin, K. R. G. et al. Gene therapy with brain-derived neurotrophic factor as a protection: retinal ganglion cells in a rat glaucoma model. Invest. Ophthalmol. Vis. Sci. 44, 4357-4365 (2003).
13. Almasieh, M., Wilson, A. M., Morquette, B., Cueva Vargas, J. L. & Di Polo, A. The molecular basis of retinal ganglion cell death in glaucoma. Prog. Retin. Eye Res. 31, 152-181 (2012).
14. Mysona, B. A., Zhao, J. & Bollinger, K. E. Role of BDNF/TrkB pathway in the visual system: Therapeutic implications for glaucoma. Expert Rev. Ophthalmol. 12, 69-81 (2017).
15. Sawai, H., Clarke, D. B., Kittlerova, P., Bray, G. M. & Aguayo, A. J. Brain-derived neurotrophic factor and neurotrophin-4/5 stimulate growth of axonal branches from regenerating retinal ganglion cells. J. Neurosci. 16, 3887-3894 (1996).
16. Zhang, Y. et al. Elevating Growth Factor Responsiveness and Axon Regeneration by Modulating Presynaptic Inputs. Neuron 103, 39-51.e5 (2019).
17. Lambuk, L. et al. Brain-Derived Neurotrophic Factor-Mediated Neuroprotection in Glaucoma: A Review of Current State of the Art. Front. Pharmacol. 13, 875662 (2022).
18. Domenici, L. et al. Rescue of retinal function by BDNF in a mouse model of glaucoma. PLOS One 9, e115579 (2014).
19. Duan, X. et al. Subtype-specific regeneration of retinal ganglion cells following axotomy: effects of osteopontin and mTOR signaling. Neuron 85, 1244-1256 (2015).
20. Kolomeyer, A. M. & Zarbin, M. A. Trophic factors in the pathogenesis and therapy for retinal degenerative diseases. Surv. Ophthalmol. 59, 134-165 (2014).
21. Huang, E. J. & Reichardt, L. F. Neurotrophins: roles in neuronal development and function. Annu. Rev. Neurosci. 24, 677-736 (2001).
22. Chao, M. V. Neurotrophins and their receptors: a convergence point for many signalling pathways. Nat. Rev. Neurosci. 4, 299-309 (2003).
23. Kaplan, D. R. & Miller, F. D. Neurotrophin signal transduction in the nervous system. Curr. Opin. Neurobiol. 10, 381-391 (2000).
24. Klein, R. et al. The trkB tyrosine protein kinase is a receptor for brain-derived neurotrophic factor and neurotrophin-3. Cell 66, 395-403 (1991).
25. English, A. W., Liu, K., Nicolini, J. M., Mulligan, A. M. & Ye, K. Small-molecule trkB agonists promote axon regeneration in cut peripheral nerves. Proc. Natl. Acad. Sci. U.S.A. 110, 16217-16222 (2013).
26. Osborne, A. et al. Neuroprotection of retinal ganglion cells by a novel gene therapy construct that achieves sustained enhancement of brain-derived neurotrophic factor/tropomyosin-related kinase receptor-B signaling. Cell Death Dis. 9, 1007 (2018).
27. Frank, L., Ventimiglia, R., Anderson, K., Lindsay, R. M. & Rudge, J. S. BDNF down-regulates neurotrophin responsiveness, TrkB protein and TrkB mRNA levels in cultured rat hippocampal neurons. Eur. J. Neurosci. 8, 1220-1230 (1996).
28. Knusel, B. et al. Ligand-induced down-regulation of Trk messenger RNA, protein and tyrosine phosphorylation in rat cortical neurons. Neuroscience 78, 851-862 (1997).
29. Sommerfeld, M. T., Schweigreiter, R., Barde, Y. A. & Hoppe, E. Down-regulation of the neurotrophin receptor TrkB following ligand binding. Evidence for an involvement of the proteasome and differential regulation of TrkA and TrkB. J. Biol. Chem. 275, 8982-8990 (2000).
30. Rabizadeh, S. et al. Induction of apoptosis by the low-affinity NGF receptor. Science 261, 345-348 (1993).
31. Chao, M. V. & Hempstead, B. L. p75 and Trk: a two-receptor system. Trends Neurosci. 18, 321-326 (1995).
32. Bamji, S. X. et al. The p75 neurotrophin receptor mediates neuronal apoptosis and is essential for naturally occurring sympathetic neuron death. J. Cell Biol. 140, 911-923 (1998).
33. Meng, L. et al. Targeting the BDNF/TrkB pathway for the treatment of tumors. Oncol. Lett. 17, 2031-2039 (2019).
34. Thiele, C. J., Li, Z. & McKee, A. E. On Trk—the TrkB signal transduction pathway is an increasingly important target in cancer biology. Clin. Cancer Res. 15, 5962-5967 (2009).
35. Colucci-D'Amato, L., Speranza, L. & Volpicelli, F. Neurotrophic Factor BDNF, Physiological Functions and Therapeutic Potential in Depression, Neurodegeneration and Brain Cancer. Int. J. Mol. Sci. 21, (2020).
36. Huang, P. et al. Optical Activation of TrkB Signaling. J. Mol. Biol. 432, 3761-3770 (2020).
37. Minichiello, L. TrkB signalling pathways in LTP and learning. Nat. Rev. Neurosci. 10, 850-860 (2009).
38. Huang, E. J. & Reichardt, L. F. Trk receptors: roles in neuronal signal transduction. Annu. Rev. Biochem. 72, 609-642 (2003).
39. Kennedy, M. J. et al. Rapid blue-light-mediated induction of protein interactions in living cells. Nat. Methods 7, 973-975 (2010).
40. Taslimi, A. et al. Optimized second-generation CRY2-CIB dimerizers and photoactivatable Cre recombinase. Nat. Chem. Biol. 12, 425-430 (2016).
41. Che, D. L., Duan, L., Zhang, K. & Cui, B. The Dual Characteristics of Light-Induced Cryptochrome 2, Homo-oligomerization and Heterodimerization, for Optogenetic Manipulation in Mammalian Cells. ACS Synth. Biol. 4, 1124-1135 (2015).
42. Duan, L. et al. Understanding CRY2 interactions for optical control of intracellular signaling. Nat. Commun. 8, 547 (2017).
43. Park, K. K. et al. Promoting axon regeneration in the adult CNS by modulation of the PTEN/mTOR pathway. Science 322, 963-966 (2008).
44. Smith, P. D. et al. SOCS3 deletion promotes optic nerve regeneration in vivo. Neuron 64, 617-623 (2009).
45. Pernet, V. et al. Long-distance axonal regeneration induced by CNTF gene transfer is impaired by axonal misguidance in the injured adult optic nerve. Neurobiol. Dis. 51, 202-213 (2013).
46. Goldberg, J. L. et al. Phase I NT-501 Ciliary Neurotrophic Factor Implant Trial for Primary Open-Angle Glaucoma: Safety, Neuroprotection, and Neuroenhancement. Ophthalmol Sci 3, 100298 (2023).
47. Hill, D., Compagnoni, C. & Cordeiro, M. F. Investigational neuroprotective compounds in clinical trials for retinal disease. Expert Opin. Investig. Drugs 30, 571-577 (2021).
48. Zhou, Y. et al. Metascape provides a biologist-oriented resource for the analysis of systems-level datasets. Nat. Commun. 10, 1523 (2019).
49. Wang, X. et al. Driving axon regeneration by orchestrating neuronal and non-neuronal innate immune responses via the IFNγ-cGAS-STING axis. Neuron 111, 236-255.e7 (2023).
50. Sarder, M., Saito, H. & Abe, K. Interleukin-2 promotes survival and neurite extension of cultured neurons from fetal rat brain. Brain Res. 625, 347-350 (1993).
51. Sarder, M., Abe, K., Saito, H. & Nishiyama, N. Comparative effect of IL-2 and IL-6 on morphology of cultured hippocampal neurons from fetal rat brain. Brain Res. 715, 9-16 (1996).
52. Yshii, L. et al. Astrocyte-targeted gene delivery of interleukin 2 specifically increases brain-resident regulatory T cell numbers and protects against pathological neuroinflammation. Nat. Immunol. 23, 878-891 (2022).
53. Mutalik, S. P. & Gupton, S. L. Glycosylation in Axonal Guidance. Int. J. Mol. Sci. 22, (2021).
54. Nogueira-Rodrigues, J. et al. Rewired glycosylation activity promotes scarless regeneration and functional recovery in spiny mice after complete spinal cord transection. Dev. Cell 57, 440-450.e7 (2022).
55. Haenold, R. et al. NF-κB controls axonal regeneration and degeneration through cell-specific balance of RelA and p50 in the adult CNS. J. Cell Sci. 127, 3052-3065 (2014).
56. Tsarouchas, T. M. et al. Dynamic control of proinflammatory cytokines Il-1ß and Tnf-α by macrophages in zebrafish spinal cord regeneration. Nat. Commun. 9, 1-17 (2018).
57. Joshi-Tope, G. et al. Reactome: a knowledgebase of biological pathways. Nucleic Acids Res. 33, D428-32 (2005).
58. Jin, L.-Q., John, B. H., Hu, J. & Selzer, M. E. Activated Erk Is an Early Retrograde Signal After Spinal Cord Injury in the Lamprey. Front. Neurosci. 14, 580692 (2020).
59. Song, Y.-S. et al. The Effects of Granulocyte-Colony Stimulating Factor on Regeneration in Nerve Crush Injuries in Rats. Neurochem. Res. 41, 1645-1650 (2016).
60. Bouhy, D. et al. Delayed GM-CSF treatment stimulates axonal regeneration and functional recovery in paraplegic rats via an increased BDNF expression by endogenous macrophages. FASEB J. 20, 1239-1241 (2006).
61. Hachem, L. D., Mothe, A. J. & Tator, C. H. Effect of BDNF and Other Potential Survival Factors in Models of In Vitro Oxidative Stress on Adult Spinal Cord-Derived Neural Stem/Progenitor Cells. Biores. Open Access 4, 146-159 (2015).
62. Siliprandi, R. et al. N-methyl-D-aspartate-induced neurotoxicity in the adult rat retina. Vis. Neurosci. 8, 567-573 (1992).
63. Guo, X. et al. Preservation of vision after CaMKII-mediated protection of retinal ganglion cells. Cell 184, 4299-4314.e12 (2021).
64. Vidal-Villegas, B. et al. MelanopsinRGCs Are fully Resistant to NMDA-Induced Excitotoxicity. Int. J. Mol. Sci. 20, (2019).
65. Christensen, I. et al. The Susceptibility of Retinal Ganglion Cells to Glutamatergic Excitotoxicity Is Type-Specific. Front. Neurosci. 13, 219 (2019).
66. Douglas, R. M. et al. Independent visual threshold measurements in the two eyes of freely moving rats and mice using a virtual-reality optokinetic system. Vis. Neurosci. 22, 677-684 (2005).
67. Koehler, C. C., Hall, L. M., Hellmer, C. B. & Ichinose, T. Using Looming Visual Stimuli to Evaluate Mouse Vision. J. Vis. Exp. (2019) doi: 10.3791/59766.
68. Yilmaz, M. & Meister, M. Rapid innate defensive responses of mice to looming visual stimuli. Curr. Biol. 23, 2011-2015 (2013).
69. Fox, M. W. The visual cliff test for the study of visual depth perception in the mouse. Anim. Behav. 13, 232-233 (1965).
70. Glynn, D., Bortnick, R. A. & Morton, A. J. Complexin II is essential for normal neurological function in mice. Hum. Mol. Genet. 12, 2431-2448 (2003).
71. Chen, S.-K., Badea, T. C. & Hattar, S. Photoentrainment and pupillary light reflex are mediated by distinct populations of ipRGCs. Nature 476, 92-95 (2011).
72. Lucas, R. J., Douglas, R. H. & Foster, R. G. Characterization of an ocular photopigment capable of driving pupillary constriction in mice. Nat. Neurosci. 4, 621-626 (2001).
73. Phase I/IIa, Open-Label, Dose-Escalation Study of Safety and Tolerability of Intravitreal RST-001 in Patients With Advanced Retinitis Pigmentosa (RP). Preprint at https://clinicaltrials.gov/study/NCT02556736 #publications.
74. Office of the Commissioner. FDA approves novel gene therapy to treat patients with a rare form of inherited vision loss. U.S. Food and Drug Administration https://www.fda.gov/news-events/press-announcements/fda-approves-novel-gene-therapy-treat-patients-rare-form-inherited-vision-loss.
75. Park, S. I. et al. Soft, stretchable, fully implantable miniaturized optoelectronic systems for wireless optogenetics. Nat. Biotechnol. 33, 1280-1286 (2015).
76. Gutruf, P. & Rogers, J. A. Implantable, wireless device platforms for neuroscience research. Curr. Opin. Neurobiol. 50, 42-49 (2018).
77. Chen, S. et al. Near-infrared deep brain stimulation via upconversion nanoparticle-mediated optogenetics. Science 359, 679-684 (2018).
78. All, A. H. et al. Expanding the Toolbox of Upconversion Nanoparticles for In Vivo Optogenetics and Neuromodulation. Adv. Mater. 31, e1803474 (2019).
79. Sun, F. et al. Sustained axon regeneration induced by co-deletion of PTEN and SOCS3. Nature 480, 372-375 (2011).
80. O'Donovan, K. J. et al. B-RAF kinase drives developmental axon growth and promotes axon regeneration in the injured mature CNS. J. Exp. Med. 211, 801-814 (2014).
81. Luo, X. et al. Enhanced Transcriptional Activity and Mitochondrial Localization of STAT3 Co-induce Axon Regrowth in the Adult Central Nervous System. Cell Rep. 15, 398-410 (2016).
82. de Gálvez, E. N. et al. The potential role of UV and blue light from the sun, artificial lighting, and electronic devices in melanogenesis and oxidative stress. J. Photochem. Photobiol. B 228, 112405 (2022).
83. Tang, F. et al. mRNA-Seq whole-transcriptome analysis of a single cell. Nat. Methods 6, 377-382 (2009).
84. Bao, S. et al. Epigenetic reversion of post-implantation epiblast to pluripotent embryonic stem cells. Nature 461, 1292-1295 (2009).
85. Picelli, S. et al. Full-length RNA-seq from single cells using Smart-seq2. Nat. Protoc. 9, 171-181 (2014).
86. Dobin, A. et al. STAR: ultrafast universal RNA-seq aligner. Bioinformatics 29, 15-21 (2013).
87. Wang, L., Wang, S. & Li, W. RSeQC: quality control of RNA-seq experiments. Bioinformatics 28, 2184-2185 (2012).
88. Liao, Y., Smyth, G. K. & Shi, W. featureCounts: an efficient general purpose program for assigning sequence reads to genomic features. Bioinformatics 30, 923-930 (2014).
89. Love, M. I., Huber, W. & Anders, S. Moderated estimation of fold change and dispersion for RNA-seq data with DESeq2. Genome Biol. 15, 550 (2014).
90. Shannon, P. et al. Cytoscape: a software environment for integrated models of biomolecular interaction networks. Genome Res. 13, 2498-2504 (2003).
91. Subramanian, A. et al. Gene set enrichment analysis: a knowledge-based approach for interpreting genome-wide expression profiles. Proc. Natl. Acad. Sci. U.S.A 102, 15545-15550 (2005).
92. Gillespie, M. et al. The reactome pathway knowledgebase 2022. Nucleic Acids Res. 50, D687-D692 (2022).

### Example 2 OptoTrkB Studies in Two Animal Models

Two animal models for ocular diseases have been used to test Opto-TrkB. First, optic nerve crush (ONC) model is used for both axon regeneration and RGC (Retinal Ganglion Cell) survival in Experiment 1: light activation for 2 hours to test the signaling activation; light activation for 2 weeks to assess how the light-gated signaling promote axon regeneration and survival; light activation for 4 weeks to assess the long term effects. Second, NMDA-induced excitotoxicity model is used in Experiment 2. ONC model is used to test Opto-AKT and Opto-SOScat in Experiment 3.

### Experiment 1. In Optic Nerve Crush Model, Light-Induced TrkB Activation Promotes RGC Survival and Axon Regeneration

We first validated light-induced TrkB activation can promote RGC survival and axon regeneration. We used AAV2-mediated gene transduction to express OptoTrkB system in mouse RGCs. Viral particles of AAV2-HA-CRY2-iTrkB (AAV2-CRY2) and AAV2-CIBN-GFP-CAAX (AAV2-CIBN) were mixed at equal amounts (0.5-1.0×10¹² titer) and intravitreally injected into 6-week old mice to allow the co-expression of the two components of OptoTrkB system. As a control, viral particles of AAV2-GFP were injected into another set of mice. Mice were subjected to optic nerve crush (ONC) four weeks after AAVs injection and were kept under blue light stimulation or in darkness for another four weeks. RGC axons in the optic nerve were anterogradely labeled by cholera toxin β subunit (CTB) 2 days before tissue analysis. An individual who was blind to different groups counted the numbers of Tuj1+RGCs and CTB-traced axons.

CTB-labeling of the optic nerves showed more significantly regenerated axons in mice injected with AAV2-CRY2+AAV2-CIBN than in mice injected with AAV2-GFP, indicating that light-activated TrkB promoted optic nerve regeneration.

Experiment 2. After NMDA Treatment (Excitotoxicity Model), Opto-Trkb Activation for 7 Days Enhanced RGCs Survival and RGCs Projection to theSC and LGN

NMDA-induced retinal cell death is a useful model to evaluate neuron damage

caused by excitotoxicity. In this experiment, we showed that light-activated TrkB signaling protects RGCs from NMDA-induced excitotoxicity. After AAVs injection, mice expressing OptoTrkB or GFP in their retinas were kept in normal circadian conditions for four weeks before retina injury by NMDA injection. Then the post-injury mice received light illumination for 1 week before tissue analysis.

A significantly larger number of RGCs survived in the retina of mice from OptoTrkB group than in GFP control group when receiving 5 mM NMDA treatment. Moreover, there were more RGCs in OptoTrkB group projected to the lateral geniculate nucleus (LGN) of thalamus and the superior colliculus (SC) in the mice, which are both targeted by RGC and play important roles in normal visual processing. These data indicate the neuroprotective effects of OptoTrkB in mice models of NMDA-induced excitotoxicity.

### Experiment 3. In Optic Nerve Crush Model, Light-Induced AKT or SOScat Activation for 2 Weeks Promotes RGC Survival and Axon Regeneration

We further explored how the downstream of ERK and AKT pathway of TrkB signaling contributed to TrkB-mediated neuroprotection. In OptoAKT1 (CIBN-GFP-CAAX+CRY2-AKT), light-induced membrane recruitment of AKT1 will elicit PI3K/AKT pathway, while in OptoSOScat (CIBN-GFP-CAAX+CRY2-SOScat) system, light-triggered membrane association of SOS catalytic domain can activate the Ras/Raf/MEK/ERK signal cascade. Mice expressing OptoAKT1, OptoSOScat, or GFP control in their retinas were subjected to ONC injury and examined by CTB-traced axons and Tuj1+labeled RGC counting.

Mice from light-stimulated OptoAKT1 group manifested significantly higher numbers of regenerated axons, while the OptoSOScat group showed milder but still evidently greater RGC axon regeneration. Both strategies promoted RGCs survival after optic nerve injury, with the OptoSoScat promoting more RGC survival.

### Example 3 Optical Activation of TrkA and TrkB Signaling

### Pre-optoTrk for Optical Activation of TrkA and TrkB Signaling In Vitro

TrkA and TrkB are highly homologous in sequence. TrkA and TrkB share similar mechanisms of activation in that binding with NGF or BDNF triggers the dimerization and autophosphorylation of the receptors (FIG. 17(II)A-B). Based on the light-gated interactions of the intracellular domain of Trk receptors (iTrk) either in the cytosol or on the cell membrane, we have constructed and compared several different methods to optically activate Trk signaling in the absence of ligand (Duan et al., Acs Synthetic Biology 2018, 7 (7): 1685-1693; Huang et al., Journal of Molecular Biology 2020, 432 (13): 3761-3770). We found that utilizing CRY2-iTrk and CIBN-CAAX with CAAX targeting the cell membrane is the most efficient one, which is denoted as pre-OptoTrk henceforth in this proposal (FIG. 17(II)A-B, FIG. 18). Upon blue light stimulation, the CRY2-CIBN association recruits iTrk to the membrane, and CRY2-CRY2 binding drives the iTrk homo-interaction on the membrane to trigger the signaling. The light-inducible signaling activation is reversible because CRY2 will dissociate from CIBN or CRY2 in the absence of blue light. We showed that in cultured cell lines, pre-OptoTrk can activate downstream signaling pathways by live-cell imaging tests and western blot assay (FIG. 18). We also demonstrated with pre-OptoTrk, light can induce the neurite outgrowth in PC12 cells (FIG. 18).

### Pre-optoTrkA can Promote Neuron Survival In Vitro

We showed that pre-OptoTrkA can support neuronal survival in vitro (FIG. 19). We used embryonic DRG neuron culture as a model system, as a large proportion of these cells cannot survive after NGF removal. DRG neurons were transfected with different optogenetic systems and then subject to NGF removal. After 48 h blue light stimulation or being incubated in the dark, the cultures were fixed and immunostained to visualize the neuron morphologies. Among different strategies, we found light-triggered TrkA signaling via pre-OptoTrkA was the most effective in promoting DRG survival, while neurons kept in the dark barely survived.

### Pre-OptoTrkB can be Successfully Expressed in RGCs in Mice and can Activate TrkB Signaling In Vivo by Light

Furthermore, we showed that the pre-OptoTrkB system can be efficiently expressed in RGCs in vivo (FIG. 20). We performed intravitreal injection for AAV2-mediated gene transfer of pre-OptoTrkB. After 4 weeks, immunostaining was conducted to examine the expression of pre-OptoTrkB in Tuj1 positive cells. More than 90% of RGCs expressed the pre-OptoTrkB system, confirming the excellent transduction rate of RGCs with our optogenetic proteins.

Next, we confirmed that light could elicit the Trk signaling in vivo in RGCs (FIG. 21). Mice receiving intravitreal injections for AAV2-mediated gene transfer of pre-OptoTrkB or GFP were kept in the dark for 4 weeks. Then while one group of mice was placed in the dark as a control, another group was exposed to 2 h blue light illumination at 100 or 400 μW/cm². The phosphorylation of the downstream signaling proteins ERK (p-ERK) and AKT (p-AKT) was probed by immunostaining (FIG. 21A, C), and the ratio of p-ERK or p-AKT positive cells was measured (FIG. 21B, D). Our results revealed that light stimulation induced a significant increase of p-ERK or p-AKT positive cells in pre-OptoTrkB-expressing mice, while under other conditions, there was no noticeable increase. What's more, exposure to different blue light intensities produced different ratios, with higher light intensity (400 μW/cm²) resulting in more p-ERK or p-AKT positive cells than lower intensity (100 μW/cm²). Therefore, our results confirmed that blue light can activate Trk signaling in RGCs in vivo, and the levels of signaling activation can be tuned by differential light intensities.

### Pre-OptoTrkB can Promote RGC Survival and Axonal Regeneration In Vivo in Mice Model of Optic Nerve Crush Injury

Moreover, we proved the optical activation of Trk signaling can promote RGC axon regeneration and cell survival after the optic nerve crush injury (FIG. 22). After AAV injections, mice expressing GFP or pre-OptoTrkB were placed in the dark for 4 weeks followed by the induction of optic nerve crush injury. Next, one group was exposed to blue light stimulation for 2 weeks while another group was kept in the dark. Mice were then sacrificed to measure the number of surviving RGCs and the lengths of regenerating axons. In mice infected with AAV-GFP, blue light stimulation did not induce a significant difference in axon regeneration or RGC survival compared with the dark control. Contrastingly, in mice expressing pre-OptoTrkB, axonal regeneration was evident, with some axons extending over 1.5 mm in the blue light stimulated group but not in the dark control group (FIG. 22A-B). Tuj1 staining showed that the rates of RGC survival increased significantly in mice receiving blue light exposure (FIG. 22C-D). The results prove that light activation of TrkB signaling can promote RGC survival and axonal regeneration in the optic nerve crush injury model of mice.

### CRY2 Mutations for Enhancing Light-Gated CRY2-CRY2 or CRY2-CIBN Interactions

We have found that C-terminal positive charges can promote the CRY2-CRY2 interaction while C-terminal negative charges suppress it37 (FIG. 23A). Based on these findings, we have developed a CRY2 variant with the E490R mutation, which bears greatly enhanced CRY2-CRY2 binding capability (FIG. 23B-C). Furthermore, we have demonstrated CRY2(E490R) can greatly increase the optical activation of intracellular signaling compared with wildtype CRY2 (CRY2 wt) (FIG. 23D). Additionally, we replaced CRY2 wt with CRY2(E490R) in the pre-OptoTrk systems. The integration of CRY2(E490R) can promote the PC12 neurite growth more effectively than CRY2 wt-based strategies (FIG. 26E-F). These results indicate that enhanced CRY2-CRY2 interaction can improve the efficiency of optogenetically controlled Trk signaling activation.

On the other hand, we revealed that CRY2 N-terminal charges are crucial for CRY2-CIB1 binding. Our data indicate that each N-terminal charged residue impacts CRY2-CIB1 dimerization differently. The Lys-2 suppresses the interaction, while Asp-4, Lys-5 and Lys-6 may promote it. By removing the positive charge at Lys-2, we have developed a CRY2 variant CRY2(Δ1-2), which possesses strongly enhanced light-gated CRY2-CIBN interaction ability (FIG. 23G-H). Moreover, in a split Cre system, we have shown that CRY2(Δ1-2) can promote the light-controlled Cre recombinase activity compared with CRY2 wt (FIG. 23I).

### Example 4 OptoSoScat and OptoAKT

### OptoSOScat

SOS (son of sevenless) is one of the RasGEF domains that activate membrane-bound Ras. The catalytic domain of the protein SOS (SOScat) can activate Ras upon recruitment to the plasma membrane. In Toettcher et al., Cell 2013, 155(6), 1422-1434, and Johnson et al., Developmental Cell 2017, 40 (2), 185-192, it was shown that light-induced membrane recruitment of SOScat can optically activate Ras/ERK pathway. See FIG. 24A, B.

We used blue light-inducible CRY2-CIBN module to recruit SOScat to the plasma membrane. This creates dimer or high-order oligomers on the membrane and activates the Ras/Raf/MEK/ERK pathway as well as the PI3K/Akt pathway. As the upstream small GTPase, Ras upon activation in turn activtaes RAF, PI3K, and other kinases. See FIG. 25A. Additional downstream signaling elements include the ERK 1/2 regulated transcription factors in the nucleus (i.e., c-Myc, c-Fos, and CREB). Upon their phosphorylation, these transcription factors act on gene expression involved in cellular growth, cell differentiation, and division.

### OptoAKT1

A fusion protein of AKT1 and CRY2 was disclosed in Katsura et al., Scientific Reports 2015, 5 (1), see FIG. 24C. Similarly, we fused the kinase domain of AKT1 (a.a. 128-480 corresponding to Uniprot P31749 AKT1_HUMAN or P31750 AKT1_MOUSE, without PH domain and disordered domain) at the C-terminus of CRY2 PHR, see FIG. 25B.

The downstream signaling elements of PI3K/AKT pathway include transcription factors of cell death genes which are suppressed by AKT. One of the well-studied examples is Forkhead family transcription factors (FOXO) which are directly phosphorylated by AKT and translocate outside of nucleus.

AKT activates downstream target mTOR, which regulates the protein synthesis necessary for cell growth, proliferation, and angiogenesis. AKT also positively regulates some transcription factors to allow the expression of pro-survival genes.

### Example 5 Ambient Light-Gated Switchable and Tunable Neuroprotection to Preserve and Restore Vision in Glaucoma

### Abstract

Glaucoma, a leading cause of irreversible blindness, is characterized by the progressive degeneration of retinal ganglion cells (RGCs) that transmit visual information to the brain. Here, we present a novel ambient light-gated therapeutic platform that uses low-intensity, long-wavelength blue light (450-490 nm) to activate TrkB signaling, a pathway critical for RGC survival and axonal regeneration. By engineering RGCs to express light-activatable TrkB receptors, the platform promotes potent axon regeneration and RGC survival following axonal injuries, matching the efficacy of well-recognized neuroprotective approaches. Standard room lighting supports axonal regeneration, while supplemental illumination further enhances neural repair. This system allows on-demand switching for adjustable treatment timing and duration, offering precise temporal control. It provides significant protection against excitotoxic injuries, preserving and even partially restoring vision in this model. In a glaucoma model, the platform mitigates RGC degeneration and preserves visual function. Strikingly, even when therapy is initiated weeks after disease onset, vision can be partially restored by promoting axon sprouting and dendrite preservation. By leveraging ambient light, this platform presents a promising, tunable, and switchable neuroprotective approach that is safe, non-invasive, and patient-friendly, with the potential to seamlessly integrate into daily life. It offers a compelling possibility for vision preservation and restoration in glaucoma and other RGC-related pathologies.

### INTRODUCTION

Glaucoma, a leading cause of irreversible blindness worldwide, is primarily characterized by the slow and progressive degeneration of retinal ganglion cells (RGCs), the sole neurons responsible for transmitting visual information from the retina to the brain. Their death results in irreversible visual loss. While elevated intraocular pressure (IOP) is a major risk factor and a key target for current treatments, RGC degeneration can occur even in cases of normal-tension glaucoma. This highlights the urgent need for neuroprotective therapies that directly protect RGCs, promote their functional recovery, and preserve or even restore vision. Complementing traditional IOP-lowering approaches, a prominent avenue for pursuing neuroprotective therapies involves activating neuroprotective signaling via sustained delivery of neurotrophic factors. A landmark progress in this domain is the entry of Ciliary Neurotrophic Factor (CNTF)-secreting ocular implant into clinical trials, which is designed to sustain ligand expression and maintain prolonged neuroprotective signaling. Despite extensive exploration, achieving functional vision recovery, even partially, remains extremely difficult. Moreover, the gradual disease progression of glaucoma, its dynamic nature, and individual variability necessitate an approach that not only provides sustained, potent protection but also enables precise, patient-specific control over the course of therapy.

The eye's transparency allows it to use light as a therapeutic tool beyond conventional molecule-based or gene therapies. For example, bright light therapy, particularly exposure to blue light, is commonly used to manage conditions like Seasonal Affective Disorder, sleep disorders, jet lag, and shift work adaptation. It leverages non-visual photoreceptive pathways in the retina to regulate biological rhythms and mood, rather than directly involving vision. While natural outdoor light is effective and sunlight more than sufficient for these therapies, specialized lamps or wearable devices, such as light-emitting glasses and visors, are readily available over the counter to supplement ambient blue light when insufficient. The success of these therapies, the simplicity and convenience to use ambient light as a therapeutic tool, and the ease to control the duration, timing and dosage, all inspire us to envision an ambient light-based neuroprotective strategy for vision preservation and restoration. Three critical challenges need to be addressed towards establishing such an ambient light-gated platform: (1) achieving robust and sustained RGC-targeted neuroprotection and effective vision restoration; (2) ensuring efficacy with low, safe doses of visible light naturally present in ambient environments; and (3) developing a switchable, reversible, and tunable therapeutic system to precisely control dosage. Such a platform could provide long-term, user-friendly treatment seamlessly integrated into daily life.

Here, we present a generalizable strategy where ambient light can activate targeted neuroprotective signaling in genetically modified RGCs. To demonstrate this strategy, we target TrkB signaling in this work. TrkB activation by Brain-Derived Neurotrophic Factor (BDNF), another important neurotrophic factor like CNTF, has been shown to protect RGC survival and enhance axon regeneration in various preclinical models. However, conventional ligand-based methods for TrkB activation, irrespective of delivery methods, are limited by receptor downregulation, nonspecific activation, and off-target effects, preventing sustained and potent RGC-targeted neuroprotection. To overcome these limitations and enable photo-activatable TrkB signaling, we screened and identified an optimized Light-inducible TrkB Activation system (LiTrkB). This system uses engineered TrkB receptors containing the intracellular domain fused with a light-sensitive protein interaction module, allowing activation by long-wavelength blue light (450-490 nm) but not ligand. Ambient blue light (0.6 mW/cm²) diffusing in the cage promoted robust RGC survival and axon regeneration, matching the effectiveness of sustained CNTF delivery, a well-recognized potent approach. This intensity is significantly lower than sunlight (12-14 mW/cm² at solar noon) and comparable to home-used white LED bulbs (up to 0.5 mW/cm²). Remarkably, even standard mouse-keeping room light (0.03 mW/cm² blue light) was adequate for axonal regeneration. By precisely modulating light intensity or duration, we achieved dose-tunable enhancement of both axon regeneration and RGC survival, providing unprecedented control over therapeutic effects. Notably, pre-injury activation significantly enhanced axonal regeneration, highlighting the importance of temporal control. Our platform preserved visual function following excitotoxic injury and partially restored vision after severe injury. In a glaucoma model, LiTrkB mitigated RGC degeneration, preserved visual function, and, more importantly, partially restored vision when therapy was initiated four weeks after disease onset. While this work focuses on TrkB signaling, the strategy provides a foundation for expanding light-based platforms to other neuroprotective pathways, opening new avenues for a convenient and effective approach to RGC neuroprotection for vision preservation and restoration.

### Results

### Identification of an Optimized Light-Gated TrkB Activation System

Blue light-gated protein-protein interaction modules provide an ideal framework for developing an ambient light-gated strategy for TrkB activation, due to four key advantages: (1) photosensory proteins, such as cryptochrome 2 (CRY2) and improved light-inducible dimer (iLID), are widely used across diverse systems and organisms, with their reliability extensively validated; (2) they can be activated by low-intensity blue light (450-490 nm), which is not only abundant in both indoor and outdoor environments, but also safer and less damaging compared to short-wavelength blue light; (3) such blue light can be easily supplemented using readily available lamps or wearable devices if needed; and (4) these light-gated interactions are reversible, with activation levels tunable by light intensity, allowing precise and flexible modulation.

We performed a direct, side-by-side comparison of three strategies for activating TrkB using either CRY2 or iLID to identify the one with best performance under low-intensity of blue light. The TrkB receptor comprises extracellular, transmembrane, and intracellular (iTrkB) domains. After binding with BDNF, receptor homo-dimerization is the key step for subsequent iTrkB autophosphorylation and downstream signaling activation. To activate TrkB optically by triggering iTrkB homo-interaction: (i) a membrane-localized iTrkB fused to CRY2 enables light-induced homo-interaction, (ii) a CRY2-CIB1 system recruits iTrkB from the cytosol to the cell membrane and facilitates CRY2-mediated homo-association, and (iii) an iLID-SspB system recruits iTrkB to the membrane and induces dimerization via tandem SspB (FIG. 26A). Downstream signaling was assessed via Western blot analysis of phosphorylated ERK1/2 and Akt in PC12 cells (FIG. 26B-D). Expression levels of iTrkB were confirmed to be equal across groups by RT-qPCR (FIG. 33A). The (ii) strategy demonstrated the highest activation efficiency, with strong light-induced activation and minimal background activity in the dark (FIG. 26B-D). Long-term functional activation was further validated using the PC12 neurite outgrowth assay. After intermittent 0.2 mW/cm² blue light exposure for 24 h, 56% of cells differentiated with type (ii), compared to 47% for type (i) and 33% for type (iii). Background differentiation in the dark was significantly lower for type (ii) (2.7%) compared to type (i) (19%) and type (iii) (9.1%) (FIG. 26E, FIG. 33B). MEK and PI3K inhibitors confirmed that p-ERK and p-Akt signaling drove light-induced neurite growth (FIG. 33C-F). These results showed that the (ii) CRY2-CIB1 strategy can most effectively activate TrkB under low blue light intensity. Therefore, we selected the type (ii) CRY2-CIB1 system (designated LiTrkB) for all subsequent studies.

Low-Intensity Blue Light Activates LiTrkb and Provides Potent Neuroprotection In Vivo after Axonal Injuries

To determine whether a low-intensity blue light could effectively activate LiTrkB and provide neuroprotection in vivo, we utilized a diffused blue light system delivering 0.6 mW/cm² from LED arrays installed on mouse cages (FIG. 26F). While 0.2 mW/cm² direct blue light is sufficient to activate TrkB in vitro, we used 0.6 mW/cm² in vivo because mice consistently move around and do not directly stare at the light source, and only a fraction of the diffused light penetrates the retina. This adjusted intensity, still significantly lower than natural sunlight (12-14 mW/cm² at solar noon), was chosen to compensate for these factors.

We tested the efficacy of this setup using the mouse optic nerve crush (ONC) injury model, a well-established system to study neuroprotection and axonal regeneration. Four weeks after AAV injection to express LiTrkB, over 90% of RGCs expressed the fusion proteins (FIG. 34A). To assess TrkB activation, mice were dark-adapted for 24 hours, then exposed to 0.6 mW/cm² blue light for 2 hours or kept in the dark before sacrifice (FIG. 34B). Mice expressing GFP were used as controls. Immunostaining revealed significantly increased p-ERK and p-Akt levels in RGCs expressing LiTrkB following blue light exposure, confirming successful light-inducible TrkB signaling in vivo (FIG. 26G-J, FIG. 34C-D). Next, we evaluated the neuroprotective and regenerative effects of LiTrkB after ONC. Mice were divided into four groups: GFP or LiTrkB expression, with or without blue light exposure for 2 weeks post-ONC (FIG. 26K). RGC survival was assessed using Tuj1 staining, while axon regeneration was evaluated by labeling RGC axons with FITC-conjugated Cholera Toxin Subunit B (CTB-FITC). In GFP groups, blue light exposure did not enhance RGC survival or axon regeneration compared to the dark group (FIG. 34E-F). In contrast, in LiTrkB-expressing mice, blue light exposure significantly improved RGC survival (FIG. 26L-M) and promoted robust axon regeneration (FIG. 26N-O). Many regenerated axons crossed the lesion site, with some extending up to 2 mm beyond it (FIG. 26N-O). In contrast, LiTrkB-expressing mice kept in the dark showed only slight axon regeneration, likely due to basal activity of LiTrkB, but no significant increase in RGC survival (FIGS. 26M and 26O).

To benchmark LiTrkB against a well-established potent neuroprotective approach, we compared it to the sustained delivery of CNTF, a treatment known for its superior neuroprotective and regenerative effects compared to other drugs, including BDNF, and currently being evaluated in human clinical trials. Our results showed that both AAV-CNTF and LiTrkB enhanced axon regeneration, with CNTF slightly outperforming LiTrkB. However, LiTrkB provided greater RGC survival compared to CNTF (FIG. 35). These findings highlight LiTrkB as a highly effective strategy for promoting RGC survival and regeneration, comparable to one of the most robust neuroprotective approaches currently available.

### The Platform Allows On-Demand Switching for Adjustable Treatment Timing and Duration

Leveraging the switchability of LiTrkB activation, we probed how different durations of TrkB signaling influenced the effect. Such temporal control relies on the reversible nature of CRY2-CRY2 and CRY2-CIBN interactions, allowing activation to cease simply by turning off blue light at any desired time point. To investigate this, mice expressing LiTrkB underwent ONC injuries four weeks after AAV injection and were randomly divided into four groups. Over the next two weeks, three groups received 0.6 mW/cm² blue light for 2, 5, or 8 days, followed by darkness, while a fourth group was treated with blue light for the full 14 days (FIG. 27A). A brief 2-day LiTrkB activation was sufficient to promote significant RGC axonal regeneration and survival compared to controls. Prolonged stimulation elicited more and longer regenerated axons (FIG. 27B-D, FIG. 36B). Interestingly, 14 days of stimulation, rather than 5 or 8 days, significantly enhanced RGC survival than 2 days (FIG. 27D, FIG. 36C), suggesting that sustained TrkB activation is required for certain RGCs populations to withstand axotomy-induced stress.

Next, we further extended blue light exposure to 4 weeks to explore the potential for long-term treatment as well as for promoting long-distance axonal regeneration, which is essential for reconnecting injured areas with distant targets, a critical yet challenging goal for clinical recovery. In the same cohort, one eye expressed GFP as a control, while the other expressed LiTrkB, with both subjected to blue light illumination (FIG. 27E). GFP-expressing eyes exhibited minimal axon regeneration and reduced RGC survival, whereas LiTrkB activation resulted in significant axonal regeneration, with some axons even reaching the optic chiasm (FIG. 27F-G). Additionally, RGC survival in the LiTrkB group was double that of the GFP group (FIG. 27H, FIG. 36E).

We also explored whether pre-injury activation of LiTrkB could enhance axonal regeneration, utilizing the platform's ability to precisely control activation timing. Two weeks after AAV-LiTrkB injection, one group received LiTrkB activation for 2 weeks prior to ONC, while a control group remained in the dark. After ONC, both groups were kept in the dark for 14 days before tissue analysis (FIG. 27I). Pre-injury LiTrkB activation significantly increased the number and length of regenerated axons compared to the control and dark groups, although the effect was not robust (FIG. 27J-K, FIG. 36G-H). Collectively, results in this section demonstrate the platform's unique ability to leverage on-demand switching, allowing precise control of treatment timing and duration to optimize outcomes.

### Adjusting Light Intensities Tunes the Level of Neuroprotection and Room Light is Sufficient to Induce Axon Regeneration

After showcasing precise temporal control of LiTrkB activation, we further explored the platform's sensitivity to and tunability by varying light intensities. Although we have found that ambient 0.6 mW/cm² blue light can provide potent neuroprotection, we investigated whether lower light intensities, such as those found in normal indoor room lighting (450-490 nm blue light ranging from 0.02-0.5 mW/cm², produced by standard home-use white LED bulbs), could induce neuroprotective effects. This study aimed to determine the sensitivity threshold of LiTrkB activation and assess its potential as a low-barrier and clinically applicable treatment option. Additionally, we evaluated whether increasing blue light intensities could tune the level of neuroprotection, offering a customizable therapeutic approach. Thus right after AAV-LiTrkB injection, mice were randomly separated into 4 groups, either kept in the dark or received blue light illumination at intensities increasing from the normal indoor lighting of the standard mouse housing room (0.03 mW/cm² blue light intensity), 0.12 mW/cm², to 0.6 mW/cm². ONC was introduced 4 weeks after AAV injection and tissue analysis was conducted 2 weeks later (FIG. 28A). Compared to the GFP group or LiTrkB dark group, mice that received room light illumination, exhibited enhanced axon regeneration of RGCs, indicating the high sensitivity of LiTrkB which enables room light-mediated neural repair (FIG. 28B-D). Increasing blue light intensity resulted in dose-dependent enhancements, with higher intensities (0.12 mW/cm² to 0.6 mW/cm²) yielding greater axon regeneration and better RGC survival rate (FIG. 28B-F). However, weak LiTrkB activation, actuated by room light, did not noticeably promote RGC survival (FIG. 28E-F), echoing the finding in FIG. 27 that pre-injury LiTrkB activation supported RGC axon regeneration rather than survival. These findings suggest that a threshold of TrkB activation may be required to promote RGC survival. The elucidation of the underlying mechanisms awaits further investigations. Taken together, our results underscore the flexibility and adjustability of our platform, which allows precise modulation of timing and light intensity to tailor TrkB activation for individualized and dynamic treatment plan.

LiTrkB Activation Protects RGC from Excitotoxic Injury Induced by NMDA

To comprehensively evaluate the neuroprotective capability of LiTrkB across different types of injuries, we utilized the NMDA-induced excitotoxicity model where RGC somas are damaged^{62,63}, complementary to ONC models that inflict injury on RGC axons. NMDA was injected into the eye 4 weeks after AAV injection (FIG. 36A). As an uninjured control, PBS was injected into mice expressing GFP. For the LiTrkB group, blue light activation started one day before NMDA injection and lasted for 15 days in total (FIG. 36A). The majority of RGCs (~83%) were dead in the injured GFP group. On the other hand, the injured group with LiTrkB activation showed nearly double the number of surviving RGCs (FIG. 36B-C). Remarkably, NMDA injection specifically caused RGC death and inner plexiform layer (IPL) thinning, with no significant changes in the thickness of other retinal layers (FIG. 37A-B). Previous research has demonstrated the resilience of αRGCs and ipRGCs to NMDA-induced damage^{64,65}. In the injured GFP group, about half of SMI32-positive αRGCs survived, while LiTrkB activation further enhanced their survival (FIG. 37C-D). In contrast, the survival of melanopsin-positive ipRGCs, inherently resistant to excitotoxic injury, remained unchanged across groups (FIG. 37E-F).

To investigate axonal preservation, Alexa Fluor 555-conjugated CTB (CTB-555) was injected into the vitreous chamber to label RGC axons and their brain projections (FIG. 29D). RGC axons in the optic nerve showed severe degeneration in the injured GFP group, while LiTrkB activation significantly preserved axons, doubling CTB intensity (FIG. 29E-F). RGC axons predominantly project to the contralateral hemisphere, including the suprachiasmatic nucleus (SCN), lateral geniculate nucleus (LGN), olivary pretectal nucleus (OPN), and superior colliculus (SC). While SCN projections, primarily from melanopsin-positive ipRGCs, were intact across groups (FIG. 29E, FIG. 38A), significant losses were observed in the LGN, OPN, and SC regions in the injured GFP group. LiTrkB activation effectively preserved projections in these areas (FIG. 29E, G-I). In summary, LiTrkB activation not only protects RGC somas but also preserves axons and brain projections from NMDA-induced excitotoxic injury.

LiTrkB Activation Preserves the Visual Function after NMDA-Induced Excitotoxic Injury

To evaluate whether these protective effects translate into functional outcomes, we investigated the ability of LiTrkB activation to preserve visual function using the NMDA-induced excitotoxicity model, which maintains the integrity of surviving RGC projections. Visual functions were assessed through a series of behavioral tests. Visual acuity, assessed by the optomotor response to rotating gratings, was severely impaired in injured GFP mice (<0.1), whereas LiTrkB activation significantly mitigated the decline, with some mice achieving values above 0.2 cycles/degree compared to uninjured controls (0.3-0.4) (FIG. 29J-K). Defensive responses to looming stimuli, such as escape or freezing, were preserved in 18 out of 23 LiTrkB-treated mice compared to only 2 out of 24 injured GFP mice, while uninjured controls responded in 23 out of 25 cases (FIG. 29L-M). Depth perception was evaluated using the visual cliff test (FIG. 29N). Although more LiTrkB-treated mice chose the shallow side than injured GFP mice, this difference was not statistically significant (FIG. 29O). However, when surveying behavior was considered, 12 out of 24 LiTrkB-treated mice avoided the deep side compared to just 1 out of 25 injured GFP mice (FIG. 29P). Additionally, unlike injured GFP mice, which rarely paused on the center platform to survey before departing, LiTrkB-treated mice spent more time surveying before leaving the platform (FIG. 29Q). Pupillary light reflex (PLR) testing revealed impaired pupil constriction in injured GFP mice (37% constriction), while LiTrkB activation improved constriction to nearly 60%, compared to over 80% in uninjured controls (FIG. 38B-C). The latency to peak constriction remained unaffected across all groups (FIG. 38D). Finally, c-Fos staining showed reduced neuronal activity in LGN, OPN, and SC regions in injured GFP mice, while LiTrkB activation significantly enhanced activity in these areas, correlating with preserved visual function (FIG. 39). In summary, LiTrkB activation preserves visual acuity, depth perception, defensive responses, PLR, and neuronal activity in RGC projection areas, effectively protecting visual function from NMDA-induced excitotoxic injury.

Late-Stage LiTrkB Activation Partially Restores Visual Functions after Excitotoxic Injury

Having established LiTrkB as a potent neuroprotective strategy, we next evaluated its therapeutic potential in a more clinically relevant scenario-intervention after substantial injury had already occurred. In the NMDA-induced excitotoxicity model, we initiated LiTrkB activation at 10 days post-injury (FIG. 30A), a timepoint when RGC degeneration had reached its peak (FIG. 40A) and significant vision loss was established. This late-stage intervention paradigm allowed us to assess whether LiTrkB can effectively control disease progression and even restore vision after significant vision damage. Ten days post-NMDA injection, 12 weeks of blue light stimulation began at this advanced disease stage. Strikingly, however, functional assessments demonstrated meaningful visual recovery. In looming response tests, NMDA-injured GFP controls exhibited minimal spontaneous recovery, with only 1 out of 12 animals responsive at 12 weeks. In contrast, LiTrkB-expressing mice demonstrated progressive functional improvement during the 12 weeks of blue light stimulation, with the number of responsive animals increasing from 2 out of 12 at 4 weeks to 7 out of 12 by the end of the stimulation period (FIG. 37B-C). This functional recovery was further corroborated by significantly improved visual acuity in optomotor response tests compared to GFP controls (FIG. 37D).

Next we further investigated the mechanisms underlying this functional recovery. Late-stage intervention failed to improve RGC survival, with comparable Tuj1-positive cell counts observed in both GFP- and LiTrkB-expressing NMDA-treated groups (FIG. 30E, FIG. 40B). Similarly, optic nerve axon intensity measurements revealed no significant preservation of axonal intensity (FIG. 30F, FIG. 40C). Then we examined terminal axon sprouting and dendritic morphology. Although the analysis of RGC terminal projections did not detect enhanced axon density in the SCN and OPN (FIG. 30F), LiTrkB activation significantly improved axon density in the contralateral LGN and SC (FIG. 30F-H), suggesting enhanced terminal sprouting that may strengthen RGC connectivity with downstream brain targets. To assess RGC dendrite preservation, we utilized Thy 1-GFP mice, in which SMI32-positive αRGCs are sparsely labeled with GFP, allowing detailed examination of dendritic morphology. LacZ expression was used as a control for LiTrkB expression. NMDA injection induced significant dendritic retraction in the injured control group. Contrasting, Sholl analysis of Thy 1-GFP-labeled RGCs demonstrated that LiTrkB restored dendritic complexity and increased dendritic branch intersections, particularly at intermediate distances (40-150 μm) from the soma (FIG. 30I-J, FIG. 40D). This dendritic regrowth likely facilitates intraretinal circuit integration between RGCs and upstream interneurons. Together, these findings indicate that enhanced terminal sprouting and dendritic regrowth contribute to partial restoration of vision.

### LiTrkB Activation Preserves RGC Survival and Vision in a Glaucoma Model

Our results demonstrate the efficacy of long-term LiTrkB activation (up to 12 weeks), showing its ability to significantly preserve and restore vision in the excitotoxicity model. This finding laid the foundation for investigating its therapeutic potential in a glaucoma model, which typically requires prolonged treatment and robust neuroprotective effects. To this end, we employed a recently established glaucoma model induced by intracameral injection of silicone oil (SO), which sustains IOP over time.

Four weeks after AAV delivery, 2 μl of SO was injected into the anterior chamber of mice expressing either GFP or LiTrkB, while PBS-injected GFP mice served as uninjured controls. Blue light stimulation began one day prior to SO injection and continued for 8 weeks (FIG. 31A). SO injection elevated IOP within 3 days, maintaining significantly higher levels for 8 weeks (FIG. 31B, FIG. 41A). Glaucoma induction caused extensive RGC degeneration, with survival rates at 4 weeks post-injection reduced to 19%, 36%, and 52% in the peripheral, mid-, and central retina, respectively. LiTrkB activation significantly mitigated this degeneration, increasing survival rates to 50%, 71%, and 76% in these regions (FIG. 31C, FIG. 41B-D). By 8 weeks, RGC degeneration worsened in GFP-expressing mice, with survival rates dropping to 9%, 19%, and 40% across the retina, whereas LiTrkB activation maintained higher survival rates of 44%, 66%, and 68%, respectively (FIG. 31D-F, FIG. 42). Notably, while untreated eyes showed a marked survival gradient favoring the central retina, LiTrkB treatment substantially reduced degeneration across all regions. Functionally, visual acuity measured by the optomotor response test declined significantly in the GFP-expressing group with SO injection to approximately 0.27 cycles/degree, compared to 0.41 cycles/degree in uninjured controls. However, visual acuity in LiTrkB-expressing mice with SO injection was preserved at 0.34 cycles/degree, significantly better than GFP controls (FIG. 31G). These results demonstrate that LiTrkB activation mitigates RGC degeneration and preserves visual function in glaucoma.

### Late-Stage LiTrkB Activation Partially Restores Visual Functions in the Glaucoma Model

We further explored the effect of LiTrkB on visual function restoration by mimicking the clinical scenario of RGC degeneration in the glaucoma model, where LiTrkB was activated after glaucoma induction. Four weeks after AAV delivery, SO was injected intracamerally to induce glaucoma in mice expressing GFP or LiTrkB, while GFP-expressing mice received PBS as an uninjured control. Four weeks later, after significant RGC degeneration had occurred (FIG. 41), LiTrkB-expressing mice were subjected to blue light stimulation for 12 weeks to activate the TrkB signaling pathway (FIG. 32A). The mice underwent the optomotor response test during this period. At 4 weeks post-SO injection, just before blue light stimulation, visual acuity significantly decreased in both the GFP- and LiTrkB-expressing mice with SO injection, with no difference between the two groups (FIG. 32B-C). At this time, RGC survival rates were similar in both groups (FIG. 43A). As glaucoma induction continued, visual acuity fluctuated in both groups, showing no significant difference until 14 weeks post-SO injection. At this point, visual acuity in the LiTrkB activation group slightly increased compared to 12 weeks post-SO injection, while it continued to decrease in the GFP-expressing group (FIG. 32B). A significant difference was observed between the GFP- and LiTrkB-expressing groups with SO injection (FIG. 32C). The difference at 16 weeks post-SO injection was even more pronounced, with visual acuity in the LiTrkB activation group maintained, while it continued to decline in the GFP-expressing group (FIG. 32B-C). These results indicate that post-injury LiTrkB activation can partially restore visual functions in the glaucoma model.

Next, we investigated the potential mechanisms underlying this partial visual function restoration. We first assessed RGC survival at 16 weeks post-SO injection. The number of surviving RGCs was similar between the GFP- and LiTrkB-expressing groups with SO injection (FIG. 32D, FIG. 43B), indicating that the visual function differences were not due to increased RGC survival in the LiTrkB activation group. We then examined terminal axon projections of RGC axons within brain targets 16 weeks post-SO injection. Glaucoma induction caused severe projection loss in the contralateral LGN and SC. However, post-injury LiTrkB activation significantly improved axon density in these regions (FIG. 32E-H). Since RGC survival rates were similar between the GFP- and LiTrkB-expressing groups with SO injection, the enhanced axon density is likely due to terminal sprouting of surviving axons. Lastly, we investigated RGC dendritic morphology within the retina. To label RGC dendrites, we injected red fluorescent pseudorabies virus (PRV-RFP) into the SC to retrogradely label RGCs (FIG. 43C). The fine structure of RGC dendrites was clearly labeled by PRV-RFP, and we focused on αRGCs, which can be identified by SMI32 staining (FIG. 43D). RGC dendrites underwent significant retraction after prolonged glaucoma induction; however, post-injury LiTrkB activation significantly enhanced RGC dendritic morphology. Sholl analysis revealed a significant increase in the number of branch intersections compared to the injured control mice (FIG. 32I-J). Thus, the partially restored visual function is likely mediated by enhanced terminal sprouting that strengthens RGC connectivity with brain targets, as well as improved RGC dendritic structures that enhance intraretinal circuit integration between RGCs and upstream interneurons.

### Discussion

This work demonstrates the potential of an ambient light-gated therapeutic platform to address the progressive degeneration of retinal ganglion cells (RGCs), a hallmark of glaucoma and related neurodegenerative conditions. By engineering RGCs to express light-activatable TrkB receptors, we showed that low-intensity, long-wavelength blue light can effectively activate TrkB signaling, a key pathway for neuronal survival and axonal regeneration. This approach not only supports axonal regeneration under standard room lighting but also enhances neural repair with supplemental illumination. The platform's ability to provide precise temporal control over treatment timing and duration represents a significant advancement, enabling on-demand modulation of neuroprotective effects. In a glaucoma model, this system successfully mitigated RGC degeneration, preserved visual function, and, notably, restored vision even when treatment was delayed until weeks after disease onset. These results emphasize the versatility and promise of this platform, particularly as a non-invasive, patient-friendly strategy that could integrate seamlessly into daily life.

The eye's transparency and accessibility to light make it an optimal organ for optogenetic interventions. A human clinical trial (NCT02556736) has been launched to investigate ion channel-based optogenetics for restoring vision in retinitis pigmentosa patients⁷³. Besides, because of the closed physical space for local gene delivery and immune privilege of the eye, ocular disorders present attractive targets for gene therapies, as demonstrated by the FDA's approval of LUXTURNA™ in 2017 for treating inherited retinal dystrophies⁷⁴. In our study, light was used as the remote switch, conferring unmatched advantages and controllability over signaling activation in RGCs. Not only can the signaling be switched on or shut down at any desired time point, but also the dosage and strength of signaling can be tuned at any time, enabling customizable and precise treatments that can be conveniently adjusted and accurately designed to meet specific or dynamically evolving needs. Thus, our ambient light-gated approach provides new possibilities and promising strategies to address the pressing need for controllable and adaptive neuroprotection.

We have shown that light-mediated TrkB signaling via LiTrkB yielded robust reparative effects in promoting both axon regeneration and survival of RGCs, which is close to the potency achieved by CNTF stimulation. Our LiTrkB method outperforms the conventional methods utilizing BDNF or other TrkB-agonists for several reasons. First, those conventional methods may be limited by insufficient expression of TrkB in some RGCs or the downregulation of TrkB induced by long-term ligand treatment^{16,19,27-29}. However, LiTrkB activation obviates the usage of ligands and does not hinge on endogenous TrkB receptors, allowing for efficient TrkB activation in neurons with little or insufficient expression of endogenous TrkB and over prolonged duration. Indeed, our results showed that 16-week LiTrkB activation is still effective and can even partially restore vision in glaucoma. Secondly, LiTrkB can produce purer neuroprotective effects than ligand-based means because it does not elicit the apoptotic p75 signaling, where p75 is the low-affinity receptor for BDNF^{22,30-32}. Thirdly, only transduced RGCs will be stimulated, not perturbing other cells in the eyes, while ligand-based methods will stimulate all the surrounding cells expressing TrkB. In the fourth place, the signaling activation of LiTrkB can be switched off at any desired time point, avoiding over-activation of TrkB signaling and thus the risks of causing cancers. LiTrkB can be potentially used to manipulate TrkB signaling in cells other than RGCs in the eye, allowing it to target other ocular disorders such as eye surface diseases and retinal diseases. In addition, it has the potential to be used to treat disorders such as stroke and spinal cord injury when supported by in vivo light delivery technologies such as small wireless LED devices or upconversion nanoparticles⁷⁵⁻⁷⁸. Thus our work sets the stage for a broader spectrum of therapeutic interventions.

Regenerating long-distance axons, a clinically critical step in connecting injured sites to distal targets, may require the sophisticated orchestration of multiple signaling pathways, such as mTOR, ERK, STAT1, and STAT3 signaling^{49,79-81}. However, manipulating several signaling pathways precisely and simultaneously remains technically challenging, which calls for the joining of new methods into the orchestra. Distinct from conventional methods that depend on the supplementation of ligands, our light-gated methods uses light, a completely orthogonal and precisely programmable physical stimulus, to activate targeted signaling pathways. Therefore, integrating optogenetics with other methods can offer another dimension of fine control. Here, we showed that combining LiTrkB activation and CNTF supplementation together triggered much more and longer regenerated axons compared to either treatment alone, especially at distances longer than 1 mm (FIG. 35). Such combinatorial approaches can undoubtedly be expanded to light-inducible control of other signaling pathways. More research is needed in the future, such as exploring orthogonal manipulations of different triggers and the mixing of different strategies/signaling pathways, to fully harness the power of optogenetics in long-distance axon regeneration.

We found that weak activation of LiTrkB did not significantly promote RGC survival, but it could facilitate mild RGC axon regeneration. This trend was reflected in several experimental conditions in our work, for example, upon weak LiTrkB activation either resulting from basal activities of LiTrkB in the dark or from activation induced by blue light of low intensity. Intriguingly, light-mediated 14-day LiTrkB activation prior to the injury produced similar results, suggesting that some residual signaling events persisted after the stop of pre-injury TrkB activation and the induction of injury. While the underlying mechanisms remained to be examined in future work, these findings showcased the capacity of our light-gated approach to unearth elusive aspects of how cells compute the information encoded by different temporal profiles and strengths of signaling for distinct cellular outcomes.

It is worth noting that the amount of blue light required for LiTrkB activation is minimal and well within the safe range. While 0.6 mW/cm² blue light was used in most experiments, 0.03 mW/cm² blue light from standard mouse housing room light was sufficient to activate LiTrkB and promote noticeable axonal regeneration (FIG. 31). Nevertheless, in our future work, varied versions of LiTrkB with different light sensitivities could be developed to require different levels of light stimulation. This would allow the selection of a specific version optimized for neuroprotection based on the lighting conditions of an individual's living environment. For cases requiring reduced activation or lower light exposure, blue light can be easily tuned down using over-the-counter blue light-filtering glasses during daytime, a simple and non-invasive solution that does not disrupt daily routines. Conversely, when higher blue light exposure is needed, increased sunlight exposure provides a natural and effective option. Supplemental devices, such as specialized lamps or wearable light-emitting glasses, can also deliver controlled blue light as needed. These tools, widely available over the counter, further support the adaptability of this platform. Together, these strategies demonstrate the flexibility of our system and its potential for seamless integration into various real-world settings, ensuring the best possible therapeutic outcomes.

### Materials and Methods

### Animals

C57BL/6J mice were obtained from Charles River. Thy 1-GFP-M (Tg(Thy1-EGFP) MJrs/J) transgenic mice were a gift from Prof. Nancy Ip at The Hong Kong University of Science and Technology and were obtained from Jackson Laboratory (Strain #: 007788. ref.). 6-8 weeks old C57BL/6J mice or Thy1-GFP mice of both genders were used in this study. All experiments were conducted in compliance with the guidelines of the Laboratory Animal Facility at the Hong Kong University of Science and Technology.

### Cloning

The details of CRY2-iTrkB and CIBN-GFP-CAAX can be found in our previous work³⁶. The intracellular domain of TrkB (a.a. 455-822, iTrkB), the photolyase homology (PHR) domain (a.a. 1-498) of Arabidopsis thaliana CRY2, and the truncated version of Arabidopsis thaliana CIB1 (a.a. 1-170) were used. For in-vitro experiments in the PC12 cell line, CRY2-iTrkB and CIBN-GFP-CAAX were inserted together with a P2A self-cleaving peptide sequence into a lentiviral vector (Addgene #11795), forming CRY2-iTrkB-P2A-CIBN-GFP-CAAX. As a control, the iTrkB gene was replaced by the mCherry (mCh) sequence to examine the effect of the optogenetic system and blue light, producing CRY2-mCh-P2A-CIBN-GFP-CAAX. For in vivo experiments, LiTrkB constructs were separately inserted into an AAV vector (Addgene #74513), generating AAV-3HA-CRY2-iTrkB and AAV-CIBN-GFP-CAAX.

### Cell Culture

PC12 cells (ATCC CRL-1721) were cultured in F-12K medium supplemented with 15% horse serum (HS) and 2.5% fetal bovine serum (FBS). HEK293T cells (ATCC CRL-3216) were cultured in DMEM supplemented with 10% fetal bovine serum. For PC12 neurite growth assay and Western blotting, PC12 cells were transduced via lentivirus and allowed to recover in a complete growth medium for another day. PC12 cells were starved in serum-reduced F-12K medium (with 1.5% HS and 0.25% FBS) overnight before being subjected to blue light stimulation. All mediums were purchased from Thermo Fisher Scientific.

### Lentiviral Transduction

LiTrkB or control constructs were inserted into a lentiviral transfer vector and were transfected into HEK293T cells together with pMD2.G and psPAX2 at 2.5:1:2 molar ratio. Media were refreshed 18 hr post transfection. Lentivirus-containing media were harvested 48 hr and 72 hr post transfection, and filtered through a 0.45 μm PES filter before being stored at −80° C. for future use.

### Western Blotting

Cells were pre-treated by 10 μM MEK inhibitor U0126 (MedChemExpress #HY-12031) and/or 20 μM PI3K inhibitor (MedChemExpress #HY-10108) for one hour before light stimulation, followed by 1 mW/cm² blue light irradiation for 10 min at 5 sec on/5 sec off intervals. After treatment, cells were rinsed with cold PBS once and lysed in lysis buffer (Beyotime P0013, supplemented with protease and phosphatase inhibitor cocktail Beyotime P1045) on ice for 15 min. Cell lysates were then transferred to a clean 1.5 mL microcentrifuge tube and spined down at 12,000 g, 4° C. for 15 mins. The protein concentrations of supernatants were determined by BCA kit (Beyotime P0010). Equal amount of protein samples was mixed with 5×SDS-PAGE sample loading buffer (Beyotime P0015) and boiled at 95° C. for 5 mins.

SDS-PAGE gels were prepared from the 10% SDS-PAGE Gel SuperQuick Preparation Kit (Beyotime P0690). Approximately 10-20 μg of protein was loaded to each lane, and gel electrophoresis was running at 90 V for around 120 mins. The protein was then transferred to a Immobilon®-P PVDF Membrane (Merck Millipore) within a Bio-rad Trans-Blot Turbo Transfer System. The blots were blocked by a 5% milk TBS-T buffer at room temperature for 1 hr. Primary antibodies were used following manufacturer's instructions: ERK1/2 (1:1000, Cell Signaling #9102), phospho-ERK1/2 (1:1000, Cell Signaling #9101), Akt (1:1000, Cell Signaling #9272), phospho-Akt (Ser473) (1:1000, Cell Signaling #4060), GAPDH (1:2500, Cell Signaling #5174).

### AAV Packaging

AAV serotype 2/2 with the hSyn promoter was utilized to overexpress GFP, LacZ, and LiTrkB genes (AAV-3HA-CRY2-iTrkB, AAV-CIBN-GFP-CAAX, or AAV-CIBN-CAAX). The virus titer was determined by qPCR and adjusted to 10¹² GC/mL for all the experiments.

### AAV Intravitreal Injection and Optic Nerve Injury

The mice were given a mixture of ketamine (80 mg/kg) and xylazine (10 mg/kg) to induce anesthesia. The conjunctiva was exposed by clamping the eyelid with an artery clamp. Two microliters of the vitreous body were withdrawn using a Hamilton syringe, followed by the gentle injection of two microliters of the virus into the eye. Eye ointment was applied to the cornea for eye protection, and postoperative analgesia was provided through the administration of ketoprofen (5 mg/kg).

Four weeks after the AAV injection, intraorbital optic nerve crush was conducted using previously described methods⁴³. The mice were anesthetized with a ketamine (80 mg/kg) and xylazine (10 mg/kg) cocktail, and an incision was made on the conjunctiva. The optic nerve was exposed and crushed using jeweler's forceps (Dumont #5; Fine Science Tools) for 2 seconds at a distance of 1-2 mm behind the optic disk. Eye ointment and ketoprofen (5 mg/kg) were applied after surgery.

### Blue Light Stimulation and Light Intensity Measurement

The mice that required blue light stimulation were housed in an individually ventilated cage (IVC). Four blue LED arrays were installed outside the walls of the case. The LED arrays were controlled by a DC power supply with a repeating pattern of 10 seconds ON and 20 seconds OFF.

The optical power meter (THORLABS, PM100D) was used to measure the intensity of light at a wavelength of 480 nm in the mouse cage. The photodiode power sensor of the power meter was positioned at the center point of the cage bottom, facing upward.

### Immunohistochemistry

The sections or whole-mount samples were incubated with the appropriate antibodies as required for the experiment. Primary antibodies: Tuj1 (1:1000, BioLegend, 801202 and 802001), p-ERK (1:500, Cell Signaling, 4370), p-AKT (1:500, Cell Signaling, 3787), FITC (1:500, Invitrogen, 71-1900), HA-Tag (1:500, Cell Signaling, 2367), GFP (1:500, Invitrogen, A10262), SMI32 (1:1000, BioLegend, 801701), Melanopsin (1:500, Advanced Targeting System, AB-N39), c-FOS (1:500, Cell Signaling, 2250), p-STAT3 (1:500, Cell Signaling, 9145), RBPMS (1:500, Proteintech, 15187-1-AP). Secondary antibodies: Goat anti-Mouse 555 (1:500, Invitrogen, A-21424), Goat anti-Mouse Cy5 (1:500, Invitrogen, A-10524), Goat anti-Rabbit 555 (1:500, Invitrogen, A-21429), Goat anti-Rabbit Cy5 (1:500, Invitrogen, A-10523), Goat anti-Chicken 488 (1:500, Invitrogen, A-11039).

To determine the number of surviving RGCs under various treatment conditions, whole-mount Tuj1 staining was performed. The mice were given a lethal dose of ketamine/xylazine and perfused with PBS followed by 4% PFA. Eyes were collected and postfixed in 4% PFA overnight before the retinas were dissected and washed with 1×PBS three times. The retinas were then incubated with Tuj1 antibody overnight at room temperature after being blocked with 0.1% Triton X-100 in 4% normal goat serum (NGS, Invitrogen, 50062Z) for 30 minutes. Following incubation with the Tuj1 antibody, the retinas were washed with PBS three times and incubated with a secondary antibody for 1 hour. The tissues were then washed with PBS and mounted onto glass slides for imaging.

For immunostaining of tissue sections, retinas, optic nerves, or brains were dissected and postfixed in 4% PFA overnight in the 4° C. refrigerator. Tissues were then cryoprotected in 30% sucrose overnight and embedded in OCT compound (SAKURA, 4583) at −80° C. Samples were sectioned at −20° C., with 25 μm for the retina and brain and 8 μm for the optic nerve. Tissue sections were then incubated with primary antibodies overnight after being blocked with 0.1% Triton X-100 in 4% NGS for 30 minutes. After the samples were washed three times with PBS, the corresponding secondary antibodies were applied for 1 hour. The samples were then washed with PBS and mounted on coverslips for imaging.

### Quantification

Whole-mount Tuj1 or RBPMS staining was performed to determine the surviving RGCs. After staining, the mounted retinas were imaged by a confocal microscope (Zeiss, LSM 880). For each retina, 12-16 images were taken from different quadrants, covering the range from the peripheral to the central regions. The number of surviving RGCs was counted by an individual who was blind to the different treatment groups.

To quantify the number of regenerated axons after optic nerve crush, CTB-FITC (Sigma-Aldrich, C1655) was intravitreally injected two days before sacrificing the mice. The collected optic nerves were cryosectioned longitudinally (section thickness: t=8 μm) and stained with the FITC antibody. The tissues were then imaged under a confocal microscope, with five images taken for each optic nerve. To quantify the number of regenerated axons at different distances from the lesion site, the following formula was used: axon number=π²×(n/d)/t. The r is the radius of the optic nerve. The n is the average axon number of the five sections. The d is the nerve width at the counting site. The t is the section thickness (8 μm).

### NMDA Treatment

Adult C57BL/6J mice were randomly separated into three groups and received AAV injections in both eyes. Two groups received an AAV-GFP injection for both eyes, while one group received an AAV-LiTrkB injection for both eyes. The mice were reared under normal circadian conditions of a daily dark-light cycle before further treatment. After 27 days, the group with double-eye AAV-LiTrkB injection received LED blue light stimulation for one day and then received double-eye 5 mM NMDA (N-Methyl-D-aspartic Acid, Sigma-Aldrich, M3262) injection. Meanwhile, one AAV-GFP group received PBS intravitreal injection as an uninjured control, and another AAV-GFP group received a 5 mM NMDA intravitreal injection. After injection, the LiTrkB group was continuously reared under blue light stimulation, while the two GFP groups were reared under normal circadian conditions. Twelve days later, mice from all three groups received CTB555 (Alexa Fluor™ 555 Conjugate, Invitrogen, C34776) intravitreal injection to trace the RGC axon projections. Two days later, the mice were terminated for further analysis. To quantify the CTB intensity in the optic nerve and RGC axonal projections in the brain, samples labeled with CTB555 from the three groups were imaged under a confocal microscope. The images were then processed and analyzed using ImageJ software. The CTB intensity was normalized by setting the intensity in the uninjured group to 100, and the intensities in the other groups were adjusted accordingly using GraphPad Prism 9 software. All visual behavior tests were conducted between 14-18 days post-NMDA/PBS treatment. The c-fos staining experiment was conducted 18 days after NMDA/PBS treatment. The mice received CTB555 intravitreal injection 2 days before termination and were dark-adapted for 10 hours before being exposed to room light for 1 hour prior to termination.

### RGC Dendrite Imaging and Sholl Analysis

Whole-mount GFP and SMI32 staining of the retina from Thy 1-GFP mice was performed to assess the dendritic morphology of αRGCs. After staining, the mounted retinas were imaged using a Zeiss LSM 880 confocal microscope, and 3D images of each RGC were obtained through z-stack imaging. The skeleton images and Sholl analysis were conducted using ImageJ and its associated plugins. The z-stack images were imported into ImageJ and flattened into 2D maximum projections to create RGC dendrite images. Dendritic arbors were semi-automatically traced using the Simple Neurite Tracer plugin, generating skeletonized images for Sholl analysis. During this analysis, concentric rings spaced at 10 μm intervals were overlaid using the Sholl Analysis plugin to quantify dendritic intersections.

### Post-Injury LiTrkB Activation in the Excitotoxic Injury Model

Adult C57BL/6J mice were randomly divided into three groups and bilaterally injected with AAVs in both eyes. Two groups received AAV-GFP injections in both eyes, while the third group received bilateral AAV-LiTrkB injections. All mice were housed under standard circadian dark-light cycle conditions. Four weeks post-AAV delivery, NMDA was intravitreally administered to both eyes of GFP- and LiTrkB-expressing mice, whereas GFP-expressing control mice received bilateral PBS injections as an uninjured control. Ten days after NMDA/PBS injections, mice were subjected to LED blue light stimulation. A looming stimulus test was performed pre-injury and weekly post-light stimulation. Visual acuity was assessed via the optomotor response test 12 weeks after light stimulation.

### Glaucoma Induction Via SO Intracameral Injection

The procedure for inducing IOP elevation in mice involves intracameral injection of SO, as described in prior studies. Mice aged 10-12 weeks are anesthetized via intraperitoneal injection of Avertin (0.3 mg/g) to avoid pupil dilation and positioned laterally on a surgical platform. A 32G needle is carefully tunneled through the corneal layers at the superotemporal limbus to access the anterior chamber without damaging the iris. Approximately 2.5 μl of SO (1,000 mPa·s, Sigma-Aldrich, 10842) is slowly injected using a sterile glass micropipette until the oil droplet covers most of the iris. The micropipette is held in place for 30 seconds post-injection to minimize leakage. The control eye receives 2.5 μl of PBS intracameral injection. Eye ointment is applied to the injected eye. Mice exhibiting corneal opacity or severe leakage of SO are excluded from analysis. All steps align with established protocols, ensuring consistency in SO-induced glaucoma modeling.

### IOP Measurement

IOP is monitored using a TonoLab tonometer (iCare, Finland) according to the provided instructions. Measurements are taken before glaucoma induction, 3 days after induction, and then weekly until the experiment is completed. Mice are anesthetized with 3% isoflurane delivered via a mouse nose cone, leaving the eyes exposed. The TonoLab device automatically records six measurements per eye, discards outliers, and generates an average reading as the final IOP value.

### PRV-RFP Retrograde Labeling

For PRV-RFP retrograde labeling, mice were anesthetized with ketamine (80 mg/kg) and xylazine (10 mg/kg) and then secured in a stereotaxic apparatus. A cranial window was created, and 0.2 μl of PRV-RFP (10¹⁰) was injected at the following coordinates: anteroposterior-3.8 mm, mediolateral 0.75 mm, and dorsoventral 1.5 mm. Thirty-six hours post-injection, the retinas were collected for histological analysis.

### Optomotor Response Test

The optomotor response tracking system (CerebralMechanics Inc. OptoMotry HD System) was used to measure the visual acuity of the mouse⁶⁶. The test was conducted by an individual who was blind to the treatment. The mouse was placed on a platform in the center of a chamber composed of four computer monitors displaying rotating sinusoidal white and black grating gradients. The grating was settled at a speed of 12 degrees per second and 100% contrast. A camera was placed above the platform to monitor the mouse's movement in real time throughout the experiment. The measurement started with a low spatial frequency (0.05 cycle/degree) after the mouse was placed on the platform and allowed to adapt for several seconds. The grating then rotated either clockwise or anticlockwise while the experimenter monitored the mouse's tracking movement for several seconds. If the mouse showed tracking behavior, the spatial frequency was gradually increased until the mouse did not show any response. The highest spatial frequency grating rotated in either direction that triggered an optomotor response from the mouse indicated the visual acuity of the corresponding eye. Each mouse was tested for approximately 10-15 minutes per session. The mean number of the two eyes represented the visual acuity of the mouse.

### Looming Stimulus Test

The looming visual stimulus response was conducted to evaluate visual function in mice⁶⁷. The test was conducted in a customized opaque chamber (45×45×30 cm) with a shelter on the side, which was 12 cm wide and mounted at a height of 8 cm. A camera was installed at a right angle to capture all areas of the chamber, and a computer monitor was set up on the top of the chamber to display a video of a black disk that expanded from 2° to 50° on a white background. The black disk was held at 2° for 250 ms before expanding to 50° in 250 ms. It then remained at 50° for 500 ms before repeating. Each expanding cycle lasted for 1s, and the test included 20 expanding repeats. Food pellets were placed outside the shelter to encourage the mouse to explore the chamber. The mouse was given ten minutes to acclimate to the chamber before the stimulus onset. A mouse was considered to have a positive response if it exhibited escape or freezing behavior upon stimulus onset.

### Visual Cliff Test

The visual cliff avoidance test was used to assess mice's depth perception and visual discrimination abilities^{69,70}. The test was conducted in a transparent acrylic box measuring 80×80×10 cm and with an opening at the top. The box was divided into two equal halves, with a platform in the center. During the test, half of the box was put on the edge of a trolley with a laminated checkerboard pattern as the shallow side. Half of the box was elevated 90 cm above a checkerboard-patterned floor as the deep side. A video camera was positioned at a suitable angle to capture the entire box and document the test. The mouse was placed on the center platform (3 cm wide). We tracked the mouse's first step off the platform to the shallow or deep side, independent of whether it explored its surroundings. We also tracked whether the mouse chose the shallow side after observing its surroundings on the platform, or if it chose either side without exploration. The mouse's exploratory behavior was detected when it probed outwardly and retracted more than twice while keeping its body on the platform. The mouse that chose to avoid the deep side after exploring was called a deep side avoider, meaning that it recognized the visual cliff and tried to avoid falling. However, if the mouse chose the shallow or deep side at random without a survey, it would not display depth perception or visual discrimination skills. In addition, we measured latency, which is the time interval between placing the mouse on the middle platform and the hindlimb leaving it. Each mouse was tested once.

### Pupillary Light Reflex Test

The pupillary light reflex test was conducted according to the established experimental procedure⁷¹ to examine non-image-forming visual function. Before the measurement, the mouse was placed in a dark room for two hours to adapt to the dark environment and to dilate the pupils to the maximum area. Then, the mice were weakly anesthetized and fixed on a mice holder to restrict their movement. The change in the mouse's pupils was recorded using an infrared camera connected to a computer. During the experiment, monochromatic light (480 nm) was used to stimulate the mouse's left eye, while the infrared camera was placed on the right eye and recorded for 60 seconds. After recording, the minimum area of the pupils after light stimulation was measured, and quantitative analysis was conducted by comparing it with the pupil area before stimulation. We also measured the latency for maximum pupil constriction, which is the time interval between the onset of light stimulation and the pupil's maximum contraction.

### Statistical Analysis

Statistical analyses were performed using GraphPad Prism 9 software. Student's t-test was used to compare two independent groups, while ANOVA followed by post-hoc multiple analyses was employed for multiple group comparisons. Fisher's exact test was utilized to compare the injured GFP group and the injured LiTrkB activation group in the looming stimulus test and the visual cliff test. The error bars in all statistical analyses represent the standard error of the mean (SEM). ns: not significant, *p≤0.05, **p≤0.01. The symbols ns, * or ** above a line indicates the comparison between the two groups that are linked by the line. If the symbols ns, * or ** are not above a line, they, by default, refer to the comparison between the corresponding group and the first group. This applies to all the statistical comparisons in this paper unless additional explanation is provided.

### REFERENCES

1. Antonetti, D. A., Silva, P. S. & Stitt, A. W. Current understanding of the molecular and cellular pathology of diabetic retinopathy. Nat. Rev. Endocrinol. 17, 195-206 (2021).
2. Weinreb, R. N., Aung, T. & Medeiros, F. A. The pathophysiology and treatment of glaucoma: a review. JAMA 311, 1901-1911 (2014).
3. Schuster, A. K., Erb, C., Hoffmann, E. M., Dietlein, T. & Pfeiffer, N. The Diagnosis and Treatment of Glaucoma. Dtsch. Arztebl. Int. 117, 225-234 (2020).
4. Wang, W. & Lo, A. C. Y. Diabetic Retinopathy: Pathophysiology and Treatments. Int. J. Mol. Sci. 19, (2018).
5. He, Z. & Jin, Y. Intrinsic Control of Axon Regeneration. Neuron 90, 437-451 (2016).
6. Liu, K., Tedeschi, A., Park, K. K. & He, Z. Neuronal intrinsic mechanisms of axon regeneration. Annu. Rev. Neurosci. 34, 131-152 (2011).
7. Boia, R. et al. Neuroprotective Strategies for Retinal Ganglion Cell Degeneration: Current Status and Challenges Ahead. Int. J. Mol. Sci. 21, (2020).
8. Fudalej, E. et al. Neuroprotective Factors of the Retina and Their Role in Promoting Survival of Retinal Ganglion Cells: A Review. Ophthalmic Res. 64, 345-355 (2021).
9. Mey, J. & Thanos, S. Intravitreal injections of neurotrophic factors support the survival of axotomized retinal ganglion cells in adult rats in vivo. Brain Res. 602, 304-317 (1993).
10. Mansour-Robaey, S., Clarke, D. B., Wang, Y. C., Bray, G. M. & Aguayo, A. J. Effects of ocular injury and administration of brain-derived neurotrophic factor on survival and regrowth of axotomized retinal ganglion cells. Proc. Natl. Acad. Sci. U.S.A 91, 1632-1636 (1994).
11. Ren, R., Li, Y., Liu, Z., Liu, K. & He, S. Long-term rescue of rat retinal ganglion cells and visual function by AAV-mediated BDNF expression after acute elevation of intraocular pressure. Invest. Ophthalmol. Vis. Sci. 53, 1003-1011 (2012).
12. Martin, K. R. G. et al. Gene therapy with brain-derived neurotrophic factor as a protection: retinal ganglion cells in a rat glaucoma model. Invest. Ophthalmol. Vis. Sci. 44, 4357-4365 (2003).
13. Almasieh, M., Wilson, A. M., Morquette, B., Cueva Vargas, J. L. & Di Polo, A. The molecular basis of retinal ganglion cell death in glaucoma. Prog. Retin. Eye Res. 31, 152-181 (2012).
14. Mysona, B. A., Zhao, J. & Bollinger, K. E. Role of BDNF/TrkB pathway in the visual system: Therapeutic implications for glaucoma. Expert Rev. Ophthalmol. 12, 69-81 (2017).
15. Sawai, H., Clarke, D. B., Kittlerova, P., Bray, G. M. & Aguayo, A. J. Brain-derived neurotrophic factor and neurotrophin-4/5 stimulate growth of axonal branches from regenerating retinal ganglion cells. J. Neurosci. 16, 3887-3894 (1996).
16. Zhang, Y. et al. Elevating Growth Factor Responsiveness and Axon Regeneration by Modulating Presynaptic Inputs. Neuron 103, 39-51.e5 (2019).
17. Lambuk, L. et al. Brain-Derived Neurotrophic Factor-Mediated Neuroprotection in Glaucoma: A Review of Current State of the Art. Front. Pharmacol. 13, 875662 (2022).
18. Domenici, L. et al. Rescue of retinal function by BDNF in a mouse model of glaucoma. PLOS One 9, e115579 (2014).
19. Duan, X. et al. Subtype-specific regeneration of retinal ganglion cells following axotomy: effects of osteopontin and mTOR signaling. Neuron 85, 1244-1256 (2015).
20. Kolomeyer, A. M. & Zarbin, M. A. Trophic factors in the pathogenesis and therapy for retinal degenerative diseases. Surv. Ophthalmol. 59, 134-165 (2014).
21. Huang, E. J. & Reichardt, L. F. Neurotrophins: roles in neuronal development and function. Annu. Rev. Neurosci. 24, 677-736 (2001).
22. Chao, M. V. Neurotrophins and their receptors: a convergence point for many signalling pathways. Nat. Rev. Neurosci. 4, 299-309 (2003).
23. Kaplan, D. R. & Miller, F. D. Neurotrophin signal transduction in the nervous system. Curr. Opin. Neurobiol. 10, 381-391 (2000).
24. Klein, R. et al. The trkB tyrosine protein kinase is a receptor for brain-derived neurotrophic factor and neurotrophin-3. Cell 66, 395-403 (1991).
25. English, A. W., Liu, K., Nicolini, J. M., Mulligan, A. M. & Ye, K. Small-molecule trkB agonists promote axon regeneration in cut peripheral nerves. Proc. Natl. Acad. Sci. U.S.A. 110, 16217-16222 (2013).
26. Osborne, A. et al. Neuroprotection of retinal ganglion cells by a novel gene therapy construct that achieves sustained enhancement of brain-derived neurotrophic factor/tropomyosin-related kinase receptor-B signaling. Cell Death Dis. 9, 1007 (2018).
27. Frank, L., Ventimiglia, R., Anderson, K., Lindsay, R. M. & Rudge, J. S. BDNF down-regulates neurotrophin responsiveness, TrkB protein and TrkB mRNA levels in cultured rat hippocampal neurons. Eur. J. Neurosci. 8, 1220-1230 (1996).
28. Knusel, B. et al. Ligand-induced down-regulation of Trk messenger RNA, protein and tyrosine phosphorylation in rat cortical neurons. Neuroscience 78, 851-862 (1997).
29. Sommerfeld, M. T., Schweigreiter, R., Barde, Y. A. & Hoppe, E. Down-regulation of the neurotrophin receptor TrkB following ligand binding. Evidence for an involvement of the proteasome and differential regulation of TrkA and TrkB. J. Biol. Chem. 275, 8982-8990 (2000).
30. Rabizadeh, S. et al. Induction of apoptosis by the low-affinity NGF receptor. Science 261, 345-348 (1993).
31. Chao, M. V. & Hempstead, B. L. p75 and Trk: a two-receptor system. Trends Neurosci. 18, 321-326 (1995).
32. Bamji, S. X. et al. The p75 neurotrophin receptor mediates neuronal apoptosis and is essential for naturally occurring sympathetic neuron death. J. Cell Biol. 140, 911-923 (1998).
33. Meng, L. et al. Targeting the BDNF/TrkB pathway for the treatment of tumors. Oncol. Lett. 17, 2031-2039 (2019).
34. Thiele, C. J., Li, Z. & McKee, A. E. On Trk—the TrkB signal transduction pathway is an increasingly important target in cancer biology. Clin. Cancer Res. 15, 5962-5967 (2009).
35. Colucci-D'Amato, L., Speranza, L. & Volpicelli, F. Neurotrophic Factor BDNF, Physiological Functions and Therapeutic Potential in Depression, Neurodegeneration and Brain Cancer. Int. J. Mol. Sci. 21, (2020).
36. Huang, P. et al. Optical Activation of TrkB Signaling. J. Mol. Biol. 432, 3761-3770 (2020).
37. Minichiello, L. TrkB signalling pathways in LTP and learning. Nat. Rev. Neurosci. 10, 850-860 (2009).
38. Huang, E. J. & Reichardt, L. F. Trk receptors: roles in neuronal signal transduction. Annu. Rev. Biochem. 72, 609-642 (2003).
39. Kennedy, M. J. et al. Rapid blue-light-mediated induction of protein interactions in living cells. Nat. Methods 7, 973-975 (2010).
40. Taslimi, A. et al. Optimized second-generation CRY2-CIB dimerizers and photoactivatable Cre recombinase. Nat. Chem. Biol. 12, 425-430 (2016).
41. Che, D. L., Duan, L., Zhang, K. & Cui, B. The Dual Characteristics of Light-Induced Cryptochrome 2, Homo-oligomerization and Heterodimerization, for Optogenetic Manipulation in Mammalian Cells. ACS Synth. Biol. 4, 1124-1135 (2015).
42. Duan, L. et al. Understanding CRY2 interactions for optical control of intracellular signaling. Nat. Commun. 8, 547 (2017).
43. Park, K. K. et al. Promoting axon regeneration in the adult CNS by modulation of the PTEN/mTOR pathway. Science 322, 963-966 (2008).
44. Smith, P. D. et al. SOCS3 deletion promotes optic nerve regeneration in vivo. Neuron 64, 617-623 (2009).
45. Pernet, V. et al. Long-distance axonal regeneration induced by CNTF gene transfer is impaired by axonal misguidance in the injured adult optic nerve. Neurobiol. Dis. 51, 202-213 (2013).
46. Goldberg, J. L. et al. Phase I NT-501 Ciliary Neurotrophic Factor Implant Trial for Primary Open-Angle Glaucoma: Safety, Neuroprotection, and Neuroenhancement. Ophthalmol Sci 3, 100298 (2023).
47. Hill, D., Compagnoni, C. & Cordeiro, M. F. Investigational neuroprotective compounds in clinical trials for retinal disease. Expert Opin. Investig. Drugs 30, 571-577 (2021).
48. Zhou, Y. et al. Metascape provides a biologist-oriented resource for the analysis of systems-level datasets. Nat. Commun. 10, 1523 (2019).
49. Wang, X. et al. Driving axon regeneration by orchestrating neuronal and non-neuronal innate immune responses via the IFNγ-cGAS-STING axis. Neuron 111, 236-255.e7 (2023).
50. Sarder, M., Saito, H. & Abe, K. Interleukin-2 promotes survival and neurite extension of cultured neurons from fetal rat brain. Brain Res. 625, 347-350 (1993).
51. Sarder, M., Abe, K., Saito, H. & Nishiyama, N. Comparative effect of IL-2 and IL-6 on morphology of cultured hippocampal neurons from fetal rat brain. Brain Res. 715, 9-16 (1996).
52. Yshii, L. et al. Astrocyte-targeted gene delivery of interleukin 2 specifically increases brain-resident regulatory T cell numbers and protects against pathological neuroinflammation. Nat. Immunol. 23, 878-891 (2022).
53. Mutalik, S. P. & Gupton, S. L. Glycosylation in Axonal Guidance. Int. J. Mol. Sci. 22, (2021).
54. Nogueira-Rodrigues, J. et al. Rewired glycosylation activity promotes scarless regeneration and functional recovery in spiny mice after complete spinal cord transection. Dev. Cell 57, 440-450.e7 (2022).
55. Haenold, R. et al. NF-κB controls axonal regeneration and degeneration through cell-specific balance of RelA and p50 in the adult CNS. J. Cell Sci. 127, 3052-3065 (2014).
56. Tsarouchas, T. M. et al. Dynamic control of proinflammatory cytokines Il-1ß and Tnf-α by macrophages in zebrafish spinal cord regeneration. Nat. Commun. 9, 1-17 (2018).
57. Joshi-Tope, G. et al. Reactome: a knowledgebase of biological pathways. Nucleic Acids Res. 33, D428-32 (2005).
58. Jin, L.-Q., John, B. H., Hu, J. & Selzer, M. E. Activated Erk Is an Early Retrograde Signal After Spinal Cord Injury in the Lamprey. Front. Neurosci. 14, 580692 (2020).
59. Song, Y.-S. et al. The Effects of Granulocyte-Colony Stimulating Factor on Regeneration in Nerve Crush Injuries in Rats. Neurochem. Res. 41, 1645-1650 (2016).
60. Bouhy, D. et al. Delayed GM-CSF treatment stimulates axonal regeneration and
functional recovery in paraplegic rats via an increased BDNF expression by endogenous macrophages. FASEB J. 20, 1239-1241 (2006).
61. Hachem, L. D., Mothe, A. J. & Tator, C. H. Effect of BDNF and Other Potential Survival Factors in Models of In Vitro Oxidative Stress on Adult Spinal Cord-Derived Neural Stem/Progenitor Cells. Biores. Open Access 4, 146-159 (2015).
62. Siliprandi, R. et al. N-methyl-D-aspartate-induced neurotoxicity in the adult rat retina. Vis. Neurosci. 8, 567-573 (1992).
63. Guo, X. et al. Preservation of vision after CaMKII-mediated protection of retinal ganglion cells. Cell 184, 4299-4314.e12 (2021).
64. Vidal-Villegas, B. et al. MelanopsinRGCs Are fully Resistant to NMDA-Induced Excitotoxicity. Int. J. Mol. Sci. 20, (2019).
65. Christensen, I. et al. The Susceptibility of Retinal Ganglion Cells to Glutamatergic Excitotoxicity Is Type-Specific. Front. Neurosci. 13, 219 (2019).
66. Douglas, R. M. et al. Independent visual threshold measurements in the two eyes of freely moving rats and mice using a virtual-reality optokinetic system. Vis. Neurosci. 22, 677-684 (2005).
67. Koehler, C. C., Hall, L. M., Hellmer, C. B. & Ichinose, T. Using Looming Visual Stimuli to Evaluate Mouse Vision. J. Vis. Exp. (2019) doi: 10.3791/59766.
68. Yilmaz, M. & Meister, M. Rapid innate defensive responses of mice to looming visual stimuli. Curr. Biol. 23, 2011-2015 (2013).
69. Fox, M. W. The visual cliff test for the study of visual depth perception in the mouse. Anim. Behav. 13, 232-233 (1965).
70. Glynn, D., Bortnick, R. A. & Morton, A. J. Complexin II is essential for normal neurological function in mice. Hum. Mol. Genet. 12, 2431-2448 (2003).
71. Chen, S.-K., Badea, T. C. & Hattar, S. Photoentrainment and pupillary light reflex are mediated by distinct populations of ipRGCs. Nature 476, 92-95 (2011).
72. Lucas, R. J., Douglas, R. H. & Foster, R. G. Characterization of an ocular photopigment capable of driving pupillary constriction in mice. Nat. Neurosci. 4, 621-626 (2001).
73. Phase I/IIa, Open-Label, Dose-Escalation Study of Safety and Tolerability of Intravitreal RST-001 in Patients With Advanced Retinitis Pigmentosa (RP). Preprint at https://clinicaltrials.gov/study/NCT02556736 #publications.
74. Office of the Commissioner. FDA approves novel gene therapy to treat patients with a rare form of inherited vision loss. U.S. Food and Drug Administration https://www.fda.gov/news-events/press-announcements/fda-approves-novel-gene-therapy-treat-patients-rare-form-inherited-vision-loss.
75. Park, S. I. et al. Soft, stretchable, fully implantable miniaturized optoelectronic systems for wireless optogenetics. Nat. Biotechnol. 33, 1280-1286 (2015).
76. Gutruf, P. & Rogers, J. A. Implantable, wireless device platforms for neuroscience research. Curr. Opin. Neurobiol. 50, 42-49 (2018).
77. Chen, S. et al. Near-infrared deep brain stimulation via upconversion nanoparticle-mediated optogenetics. Science 359, 679-684 (2018).
78. All, A. H. et al. Expanding the Toolbox of Upconversion Nanoparticles for In Vivo Optogenetics and Neuromodulation. Adv. Mater. 31, e1803474 (2019).
79. Sun, F. et al. Sustained axon regeneration induced by co-deletion of PTEN and SOCS3. Nature 480, 372-375 (2011).
80. O'Donovan, K. J. et al. B-RAF kinase drives developmental axon growth and promotes axon regeneration in the injured mature CNS. J. Exp. Med. 211, 801-814 (2014).
81. Luo, X. et al. Enhanced Transcriptional Activity and Mitochondrial Localization of STAT3 Co-induce Axon Regrowth in the Adult Central Nervous System. Cell Rep. 15, 398-410 (2016).
82. de Gálvez, E. N. et al. The potential role of UV and blue light from the sun, artificial lighting, and electronic devices in melanogenesis and oxidative stress. J. Photochem. Photobiol. B 228, 112405 (2022).
83. Tang, F. et al. mRNA-Seq whole-transcriptome analysis of a single cell. Nat. Methods 6, 377-382 (2009).
84. Bao, S. et al. Epigenetic reversion of post-implantation epiblast to pluripotent embryonic stem cells. Nature 461, 1292-1295 (2009).
85. Picelli, S. et al. Full-length RNA-seq from single cells using Smart-seq2. Nat. Protoc. 9, 171-181 (2014).
86. Dobin, A. et al. STAR: ultrafast universal RNA-seq aligner. Bioinformatics 29, 15-21 (2013).
87. Wang, L., Wang, S. & Li, W. RSeQC: quality control of RNA-seq experiments. Bioinformatics 28, 2184-2185 (2012).
88. Liao, Y., Smyth, G. K. & Shi, W. featureCounts: an efficient general purpose program for assigning sequence reads to genomic features. Bioinformatics 30, 923-930 (2014).
89. Love, M. I., Huber, W. & Anders, S. Moderated estimation of fold change and dispersion for RNA-seq data with DESeq2. Genome Biol. 15, 550 (2014).
90. Shannon, P. et al. Cytoscape: a software environment for integrated models of biomolecular interaction networks. Genome Res. 13, 2498-2504 (2003).
91. Subramanian, A. et al. Gene set enrichment analysis: a knowledge-based approach for interpreting genome-wide expression profiles. Proc. Natl. Acad. Sci. U.S.A 102, 15545-15550 (2005).
92. Gillespie, M. et al. The reactome pathway knowledgebase 2022. Nucleic Acids Res. 50, D687-D692 (2022).

All patents, patent applications, and other publications, including GenBank Accession Numbers and the like, cited in this application are incorporated by reference in the entirety for all purposes.

Sequence Listing

SEQ ID NO: 1 amino acid sequence of wild-type CRY2 protein
MKMDKKTIVWFRRDLRIEDNPALAAAAHEGSVFPVFIWCPEEEGQFYPGRASRWW
MKQSLAHLSQSLKALGSDLTLIKTHNTISAILDCIRVTGATKVVFNHLYDPVSLVRDH
TVKEKLVERGISVQSYNGDLLYEPWEIYCEKGKPFTSFNSYWKKCLDMSIESVMLPP
PWRLMPITAAAEAIWACSIEELGLENEAEKPSNALLTRAWSPGWSNADKLLNEFIEK
QLIDYAKNSKKVVGNSTSLLSPYLHFGEISVRHVFQCARMKQIIWARDKNSEGEESA
DLFLRGIGLREYSRYICFNFPFTHEQSLLSHLRFFPWDADVDKFKAWRQGRTGYPLV
DAGMRELWATGWMHNRIRVIVSSFAVKFLLLPWKWGMKYFWDTLLDADLECDILG
WQYISGSIPDGHELDRLDNPALQGAKYDPEGEYIRQWLPELARLPTEWIHHPWDAPL
TVLKASGVELGTNYAKPIVDIDTARELLAKAISRTREAQIMIGAAPDEIVADSFEALG
ANTIKEPGLCPSVSSNDQQVPSAVRYNGSKRVKPEEEEERDMKKSRGFDERELFSTA
ESSSSSSVFFVSQSCSLASEGKNLEGIQDSSDQITTSLGKNGCK
SEQ ID NO: 2 amino acid sequence of full-length rat TrkB protein (underlined is the
portion of iTrkB used in SEQ ID NO: 3 Opto-TrkB fusion protein)
MSPWPRWHGPAMARLWGLCLLVLGFWRASLACPMSCKCSTTRIWCTEPSPGIVAFPRLEPN
SIDPENITEILIANQKRLEIINEDDVEAYVGLKNLTIVDSGLKFVAYKAFLKNGNLRHINFTRNKLTS
LSRRHFRHLDLSDLILTGNPFTCSCDIMWLKTLQETKSSPDTQDLYCLNESSKNTPLANLQIPN
CGLPSARLAAPNLTVEEGKSVTISCSVGGDPLPTLYWDVGNLVSKHMNETSHTQGSLRITNISS
DDSGKQISCVAENLVGEDQDSVNLTVHFAPTITFLESPTSDHHWCIPFTVRGNPKPALQWFYN
GAILNESKYICTKIHVTNHTEYHGCLQLDNPTHMNNGDYTLMAKNEYGKDERQISAHFMGRP
GVDYETNPNYPEVLYEDWTTPTDIGDTTNKSNEIPSTDVADQTNREHLSVYAVVVIASVVGFCL
LVMLLLLKLARHSKFGMKGPASVISNDDDSASPLHHISNGSNTPSSSEGGPDAVIIGMTKIPVIE
NPQYFGITNSQLKPDTFVQHIKRHNIVLKRELGEGAFGKVFLAECYNLCPEQDKILVAVKTLKDA
SDNARKDFHREAELLTNLQHEHIVKFYGVCVEGDPLIMVFEYMKHGDLNKFLRAHGPDAVLM
AEGNPPTELTQSQMLHIAQQIAAGMVYLASQHFVHRDLATRNCLVGENLLVKIGDFGMSRDV
YSTDYYRVGGHTMLPIRWMPPESIMYRKETTESDVWSLGVVLWEIFTYGKQPWYQLSNNEVIE
CITQGRVLQRPRTCPQEVYELMLGCWQREPHTRKNIKNIHTLLQNLAKASPVYLDILG
SEQ ID NO: 3 amino acid sequence of Opto-TrkB fusion protein (3HA-CRY2-iTrkB)
MYPYDVPDYAGYPYDVPDYAGYPYDVPDYAGMKMDKKTIVWFRRDLRIEDNPALAA
AAHEGSVFPVFIWCPEEEGQFYPGRASRWWMKQSLAHLSQSLKALGSDLTLIK
THNTISAILDCIRVTGATKVVFNHLYDPVSLVRDHTVKEKLVERGISVQSYNGDL
LYEPWEIYCEKGKPFTSFNSYWKKCLDMSIESVMLPPPWRLMPITAAAEAIWAC
SIEELGLENEAEKPSNALLTRAWSPGWSNADKLLNEFIEKQLIDYAKNSKKVVG
NSTSLLSPYLHFGEISVRHVFQCARMKQIIWARDKNSEGEESADLFLRGIGLREY
SRYICFNFPFTHEQSLLSHLRFFPWDADVDKFKAWRQGRTGYPLVDAGMRELW
ATGWMHNRIRVIVSSFAVKFLLLPWKWGMKYFWDTLLDADLECDILGWQYIS
GSIPDGHELDRLDNPALQGAKYDPEGEYIRQWLPELARLPTEWIHHPWDAPLT
VLKASGVELGTNYAKPIVDIDTARELLAKAISRTREAQIMIGAARGGSDIKLARHS
KFGMKGPASVISNDDDSASPLHHISNGSNTPSSSEGGPDAVIIGMTKIPVIENPQYFGIT
NSQLKPDTFVQHIKRHNIVLKRELGEGAFGKVFLAECYNLCPEQDKILVAVKTLKDA
SDNARKDFHREAELLTNLQHEHIVKFYGVCVEGDPLIMVFEYMKHGDLNKFLRAHG
PDAVLMAEGNPPTELTQSQMLHIAQQIAAGMVYLASQHFVHRDLATRNCLVGENLL
VKIGDFGMSRDVYSTDYYRVGGHTMLPIRWMPPESIMYRKFTTESDVWSLGVVLWE
IFTYGKQPWYQLSNNEVIECITQGRVLQRPRTCPQEVYELMLGCWQREPHMRKNIKG
IHTLLQNLAKASPVYLDILG
SEQ ID NO: 4 amino acid sequence of full-length CIB1 protein (underlined is CIBN used in
the Examples)
MNGAIGGDLLLNFPDMSVLERQRAHLKYLNPTFDSPLAGFFADSSMITGGEMDSYLS
TAGLNLPMMYGETTVEGDSRLSISPETTLGTGNFKKRKFDTETKDCNEKKKKMTMN
RDDLVEEGEEEKSKITEQNNGSTKSIKKMKHKAKKEENNFSNDSSKVTKELEKTDYI
HVRARRGQATDSHSIAERVRREKISERMKFLQDLVPGCDKITGKAGMLDEIINYVQS
LQRQIEFLSMKLAIVNPRPDFDMDDIFAKEVASTPMTVVPSPEMVLSGYSHEMVHSG
YSSEMVNSGYLHVNPMQQVNTSSDPLSCFNNGEAPSMWDSHVQNLYGNLGV
SEQ ID NO: 5 amino acid sequence of CAAX
GKKKKKKSKTKCVIM
SEQ ID NO: 6 amino acid sequence of CIBN-GFP-CAAX fusion protein (CIBN-GFP-CAAX)
MNGAIGGDLLLNFPDMSVLERQRAHLKYLNPTFDSPLAGFFADSSMITGGEMDSYLS
TAGLNLPMMYGETTVEGDSRLSISPETTLGTGNFKAAKFDTETKDCNEAAKKMTMN
RDDLVEEGEEEKSKITEQNNGSTKSIKKMKHKAKKEENNFSNDSSKVTKELEKTDYI
HVPVATMVSKGEELFTGVVPILVELDGDVNGHKFSVSGEGEGDATYGKLTLKFI
CTTGKLPVPWPTLVTTLTYGVQCFSRYPDHMKQHDFFKSAMPEGYVQERTIFF
KDDGNYKTRAEVKFEGDTLVNRIELKGIDFKEDGNILGHKLEYNYNSHNVYIMA
DKQKNGIKVNFKIRHNIEDGSVQLADHYQQNTPIGDGPVLLPDNHYLSTQSALS
KDPNEKRDHMVLLEFVTAAGITLGMDELYKGKKKKKKSKTKCVIM
SEQ ID NO: 7 amino acid sequence of full-length human TrkB protein (iTrkB underlined)
MSSWIRWHGPAMARLWGFCWLVVGFWRAAF ACPTSCKCSASRIWCSDPSPGIVAFP
RLEPNSVDPENITEIFIANQKRLEIINEDDVEAYVGLRNLTIVDSGLKFVAHKAFLKNS
NLQHINFTRNKLTSLSRKHFRHLDLSELILVGNPFTCSCDIMWIKTLQEAKSSPDTQDL
YCLNESSKNIPLANLQIPNCGLPSANLAAPNLTVEEGKSITLSCSVAGDPVPNMYWDV
GNLVSKHMNETSHTQGSLRITNISSDDSGKQISCVAENLVGEDQDSVNLTVHFAPTIT
FLESPTSDHHWCIPFTVKGNPKPALQWFYNGAILNESKYICTKIHVINHTEYHGCLQL
DNPTHMNNGDYTLIAKNEYGKDEKQISAHFMGWPGIDDGANPNYPDVIYEDYGTA
ANDIGDTTNRSNEIPSTDVTDKTGREHLSVYAVVVIASVVGFCLLVMLFLLKLARHS
KFGMKGPASVISNDDDSASPLHHISNGSNTPSSSEGGPDAVIIGMTKIPVIENPQYFGIT
NSQLKPDTFVQHIKRHNIVLKRELGEGAFGKVFLAECYNLCPEQDKILVAVKTLKDA
SDNARKDFHREAELLTNLQHEHIVKFYGVCVEGDPLIMVFEYMKHGDLNKFLRAHG
PDAVLMAEGNPPTELTQSQMLHIAQQIAAGMVYLASQHFVHRDLATRNCLVGENLL
VKIGDFGMSRDVYSTDYYRVGGHTMLPIRWMPPESIMYRKFTTESDVWSLGVVLWE
IFTYGKQPWYQLSNNEVIECITQGRVLQRPRTCPQEVYELMLGCWQREPHMRKNIKG
IHTLLQNLAKASPVYLDILG

1. A fusion protein comprising an optic-responsive element and a downstream signaling element derived from a domain of a signaling molecule through which the signaling molecule activates its downstream effector molecules, wherein upon exposure to a light within a pre-determined wavelength the fusion protein activates a downstream signal transduction pathway mediated by the signaling molecule, and wherein the optic-responsive element is a modified Arabidopsis thaliana cryptochrome 2 (CRY2) comprising any one or two or more of the mutations independently selected from the group consisting of:
(1) a deletion of the first and second amino acids of N-terminus of CRY2;
(2) a K2S substitution;
(3) an E281A substitution; and
(4) an E490R substitution.

2. The fusion protein of claim 1, wherein the downstream signaling element is derived from a receptor protein's intracellular domain.

3. The fusion protein of claim 2, wherein the downstream signaling element comprises the intracellular domain of tropomyosin receptor kinase B (TrkB).

4. The fusion protein of claim 1, wherein the downstream signaling element is derived from a catalytic domain of the signaling molecule.

5. The fusion protein of claim 4, wherein the downstream signaling element comprises the catalytic domain of SOS protein (SOScat).

6. The fusion protein of claim 4, wherein the downstream signaling element comprises the kinase domain of AKT1.

7. The fusion protein of claim 1, further comprising a cell membrane-targeting element.

8. The fusion protein of claim 7, wherein the cell membrane-targeting element is an N-terminal Lyn tag.

9. The fusion protein of claim 7, wherein the cell membrane-targeting element is a CAAX motif.

10. The fusion protein of claim 1, which does not comprise a cell membrane-targeting element.

11. The fusion protein of claim 1, further comprising a detectable moiety.

12. A nucleic acid comprising a polynucleotide sequence encoding the fusion protein of claim 1.

13-14. (canceled)

15. A composition comprising (1) the fusion protein of claim 1 or a nucleic acid comprising a polynucleotide sequence encoding the fusion protein; and (2) at least one physiologically acceptable excipient.

16. A host cell comprising the fusion protein of claim 1 or the polynucleotide sequence encoding the fusion protein.

17-20. (canceled)

21. A method for activating signal transduction, comprising:
(i) obtaining a cell comprising the fusion protein of claim 1; and
(ii) exposing the cell to a light within a pre-determined wavelength range, thereby activating the receptor protein's downstream signal transduction pathway.

22. The method of claim 21, wherein the activating of the receptor protein's downstream signal transduction pathway results in proliferation, survival, and/or regeneration of the cell.

23. The method of claim 22, wherein the downstream signaling element comprises intracellular domain of tropomyosin receptor kinase B (TrkB), wherein the cell is a human optic neuronal cell, and wherein the activating of downstream signal transduction pathway results in proliferation, survival, and/or regeneration of the human optic neuronal cell.

24. The method of claim 23, wherein the human optic neuronal cell is within a human eye.

25. The method of claim 23, wherein the fusion protein does not comprise a cell membrane-targeting element, and wherein the cell further comprises a second fusion protein comprising a CAAX motif and CIBN.

26. The method of claim 23, wherein the fusion protein comprises an N-terminal Lyn tag as the cell membrane-targeting element.

27-28. (canceled)


SEQUENCE LISTING (20 sequences)

SEQ ID NO: 1 - protein, 612 aa, Homo sapiens
  REGION 1..612: note = Description of sequence: amino acid sequence of wild-type CRY2 protein
MKMDKKTIVWFRRDLRIEDNPALAAAAHEGSVFPVFIWCPEEEGQFYPGRASRWWMKQSLAHLSQSLKALGSDLTLIKTHNTISAILDCIRVTGATKVVFNHLYDPVSLVRDHTVKEKLVERGISVQSYNGDLLYEPWEIYCEKGKPFTSFNSYWKKCLDMSIESVMLPPPWRLMPITAAAEAIWACSIEELGLENEAEKPSNALLTRAWSPGWSNADKLLNEFIEKQLIDYAKNSKKVVGNSTSLLSPYLHFGEISVRHVFQCARMKQIIWARDKNSEGEESADLFLRGIGLREYSRYICFNFPFTHEQSLLSHLRFFPWDADVDKFKAWRQGRTGYPLVDAGMRELWATGWMHNRIRVIVSSFAVKFLLLPWKWGMKYFWDTLLDADLECDILGWQYISGSIPDGHELDRLDNPALQGAKYDPEGEYIRQWLPELARLPTEWIHHPWDAPLTVLKASGVELGTNYAKPIVDIDTARELLAKAISRTREAQIMIGAAPDEIVADSFEALGANTIKEPGLCPSVSSNDQQVPSAVRYNGSKRVKPEEEEERDMKKSRGFDERELFSTAESSSSSSVFFVSQSCSLASEGKNLEGIQDSSDQITTSLGKNGCK

SEQ ID NO: 2 - protein, 821 aa, Rattus norvegicus
  REGION 1..821: note = Description of sequence: amino acid sequence of full-length rat TrkB protein
  REGION 454..821: note = the portion of iTrkB used in the Opto-TrkB fusion protein
MSPWPRWHGPAMARLWGLCLLVLGFWRASLACPMSCKCSTTRIWCTEPSPGIVAFPRLEPNSIDPENITEILIANQKRLEIINEDDVEAYVGLKNLTIVDSGLKFVAYKAFLKNGNLRHINFTRNKLTSLSRRHFRHLDLSDLILTGNPFTCSCDIMWLKTLQETKSSPDTQDLYCLNESSKNTPLANLQIPNCGLPSARLAAPNLTVEEGKSVTISCSVGGDPLPTLYWDVGNLVSKHMNETSHTQGSLRITNISSDDSGKQISCVAENLVGEDQDSVNLTVHFAPTITFLESPTSDHHWCIPFTVRGNPKPALQWFYNGAILNESKYICTKIHVTNHTEYHGCLQLDNPTHMNNGDYTLMAKNEYGKDERQISAHFMGRPGVDYETNPNYPEVLYEDWTTPTDIGDTTNKSNEIPSTDVADQTNREHLSVYAVVVIASVVGFCLLVMLLLLKLARHSKFGMKGPASVISNDDDSASPLHHISNGSNTPSSSEGGPDAVIIGMTKIPVIENPQYFGITNSQLKPDTFVQHIKRHNIVLKRELGEGAFGKVFLAECYNLCPEQDKILVAVKTLKDASDNARKDFHREAELLTNLQHEHIVKFYGVCVEGDPLIMVFEYMKHGDLNKFLRAHGPDAVLMAEGNPPTELTQSQMLHIAQQIAAGMVYLASQHFVHRDLATRNCLVGENLLVKIGDFGMSRDVYSTDYYRVGGHTMLPIRWMPPESIMYRKFTTESDVWSLGVVLWEIFTYGKQPWYQLSNNEVIECITQGRVLQRPRTCPQEVYELMLGCWQREPHTRKNIKNIHTLLQNLAKASPVYLDILG

SEQ ID NO: 3 - protein, 903 aa, synthetic construct
  REGION 1..903: note = Description of sequence: amino acid sequence of Opto-TrkB fusion protein
  REGION 1..31: note = 3HA sequence
  REGION 32..529: note = CRY2 Sequence
  REGION 534..903: note = iTrkB sequence
MYPYDVPDYAGYPYDVPDYAGYPYDVPDYAGMKMDKKTIVWFRRDLRIEDNPALAAAAHEGSVFPVFIWCPEEEGQFYPGRASRWWMKQSLAHLSQSLKALGSDLTLIKTHNTISAILDCIRVTGATKVVFNHLYDPVSLVRDHTVKEKLVERGISVQSYNGDLLYEPWEIYCEKGKPFTSFNSYWKKCLDMSIESVMLPPPWRLMPITAAAEAIWACSIEELGLENEAEKPSNALLTRAWSPGWSNADKLLNEFIEKQLIDYAKNSKKVVGNSTSLLSPYLHFGEISVRHVFQCARMKQIIWARDKNSEGEESADLFLRGIGLREYSRYICFNFPFTHEQSLLSHLRFFPWDADVDKFKAWRQGRTGYPLVDAGMRELWATGWMHNRIRVIVSSFAVKFLLLPWKWGMKYFWDTLLDADLECDILGWQYISGSIPDGHELDRLDNPALQGAKYDPEGEYIRQWLPELARLPTEWIHHPWDAPLTVLKASGVELGTNYAKPIVDIDTARELLAKAISRTREAQIMIGAARGGSDIKLARHSKFGMKGPASVISNDDDSASPLHHISNGSNTPSSSEGGPDAVIIGMTKIPVIENPQYFGITNSQLKPDTFVQHIKRHNIVLKRELGEGAFGKVFLAECYNLCPEQDKILVAVKTLKDASDNARKDFHREAELLTNLQHEHIVKFYGVCVEGDPLIMVFEYMKHGDLNKFLRAHGPDAVLMAEGNPPTELTQSQMLHIAQQIAAGMVYLASQHFVHRDLATRNCLVGENLLVKIGDFGMSRDVYSTDYYRVGGHTMLPIRWMPPESIMYRKFTTESDVWSLGVVLWEIFTYGKQPWYQLSNNEVIECITQGRVLQRPRTCPQEVYELMLGCWQREPHMRKNIKGIHTLLQNLAKASPVYLDILG

SEQ ID NO: 4 - protein, 335 aa, Homo sapiens
  REGION 1..335: note = Description of sequence: amino acid sequence of full-length CIB1 protein
  REGION 1..170: note = CIBN
MNGAIGGDLLLNFPDMSVLERQRAHLKYLNPTFDSPLAGFFADSSMITGGEMDSYLSTAGLNLPMMYGETTVEGDSRLSISPETTLGTGNFKKRKFDTETKDCNEKKKKMTMNRDDLVEEGEEEKSKITEQNNGSTKSIKKMKHKAKKEENNFSNDSSKVTKELEKTDYIHVRARRGQATDSHSIAERVRREKISERMKFLQDLVPGCDKITGKAGMLDEIINYVQSLQRQIEFLSMKLAIVNPRPDFDMDDIFAKEVASTPMTVVPSPEMVLSGYSHEMVHSGYSSEMVNSGYLHVNPMQQVNTSSDPLSCFNNGEAPSMWDSHVQNLYGNLGV

SEQ ID NO: 5 - protein, 15 aa, unidentified
  REGION 1..15: note = Description of sequence: amino acid sequence of CAAX
GKKKKKKSKTKCVIM

SEQ ID NO: 6 - protein, 430 aa, synthetic construct
  REGION 1..430: note = Description of sequence: amino acid sequence of CIBN-GFP-CAAX fusion protein
  REGION 1..170: note = CIBN
  REGION 177..415: note = GFP
  REGION 416..430: note = CAAX
MNGAIGGDLLLNFPDMSVLERQRAHLKYLNPTFDSPLAGFFADSSMITGGEMDSYLSTAGLNLPMMYGETTVEGDSRLSISPETTLGTGNFKAAKFDTETKDCNEAAKKMTMNRDDLVEEGEEEKSKITEQNNGSTKSIKKMKHKAKKEENNFSNDSSKVTKELEKTDYIHVPVATMVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYKGKKKKKKSKTKCVIM

SEQ ID NO: 7 - protein, 822 aa, Homo sapiens
  REGION 1..822: note = Description of sequence: amino acid sequence of full-length human TrkB protein
  REGION 455..822: note = iTrkB
MSSWIRWHGPAMARLWGFCWLVVGFWRAAFACPTSCKCSASRIWCSDPSPGIVAFPRLEPNSVDPENITEIFIANQKRLEIINEDDVEAYVGLRNLTIVDSGLKFVAHKAFLKNSNLQHINFTRNKLTSLSRKHFRHLDLSELILVGNPFTCSCDIMWIKTLQEAKSSPDTQDLYCLNESSKNIPLANLQIPNCGLPSANLAAPNLTVEEGKSITLSCSVAGDPVPNMYWDVGNLVSKHMNETSHTQGSLRITNISSDDSGKQISCVAENLVGEDQDSVNLTVHFAPTITFLESPTSDHHWCIPFTVKGNPKPALQWFYNGAILNESKYICTKIHVTNHTEYHGCLQLDNPTHMNNGDYTLIAKNEYGKDEKQISAHFMGWPGIDDGANPNYPDVIYEDYGTAANDIGDTTNRSNEIPSTDVTDKTGREHLSVYAVVVIASVVGFCLLVMLFLLKLARHSKFGMKGPASVISNDDDSASPLHHISNGSNTPSSSEGGPDAVIIGMTKIPVIENPQYFGITNSQLKPDTFVQHIKRHNIVLKRELGEGAFGKVFLAECYNLCPEQDKILVAVKTLKDASDNARKDFHREAELLTNLQHEHIVKFYGVCVEGDPLIMVFEYMKHGDLNKFLRAHGPDAVLMAEGNPPTELTQSQMLHIAQQIAAGMVYLASQHFVHRDLATRNCLVGENLLVKIGDFGMSRDVYSTDYYRVGGHTMLPIRWMPPESIMYRKFTTESDVWSLGVVLWEIFTYGKQPWYQLSNNEVIECITQGRVLQRPRTCPQEVYELMLGCWQREPHMRKNIKGIHTLLQNLAKASPVYLDILG

SEQ ID NO: 8 - protein, 9 aa, Arabidopsis thaliana
  REGION 1..9: note = Description of sequence: Peptide, name: CRY2wt
MKMDKKTIV

SEQ ID NO: 9 - protein, 7 aa, Arabidopsis thaliana
  REGION 1..7: note = Description of sequence: Peptide, name: CRY2wt (Delta 1-2)
MDKKTIV

SEQ ID NO: 10 - protein, 5 aa, Arabidopsis thaliana
  REGION 1..5: note = Description of sequence: Peptide, name: CRY2wt (Delta 1-4)
KKTIV

SEQ ID NO: 11 - protein, 4 aa, Arabidopsis thaliana
  REGION 1..4: note = Description of sequence: Peptide, name: CRY2wt (Delta 1-5)
KTIV

SEQ ID NO: 12 - skipped

SEQ ID NO: 13 - protein, 9 aa, Arabidopsis thaliana
  REGION 1..9: note = Description of sequence: Peptide, name: CRY(K2D)
MDMDKKTIV

SEQ ID NO: 14 - protein, 9 aa, Arabidopsis thaliana
  REGION 1..9: note = Description of sequence: Peptide, name: CRY2(K2E)
MEMDKKTIV

SEQ ID NO: 15 - protein, 9 aa, Arabidopsis thaliana
  REGION 1..9: note = Description of sequence: Peptide, name: CRY2(D4H)
MKMHKKTIV

SEQ ID NO: 16 - protein, 9 aa, Arabidopsis thaliana
  REGION 1..9: note = Description of sequence: Peptide, name: CRY2(D4R)
MKMRKKTIV

SEQ ID NO: 17 - protein, 9 aa, Arabidopsis thaliana
  REGION 1..9: note = Description of sequence: Peptide, name: CRY2(K5D)
MKMDDKTIV

SEQ ID NO: 18 - protein, 9 aa, Arabidopsis thaliana
  REGION 1..9: note = Description of sequence: Peptide, name: CRY2(K5E)
MKMDEKTIV

SEQ ID NO: 19 - protein, 9 aa, Arabidopsis thaliana
  REGION 1..9: note = Description of sequence: Peptide, name: CRY2(K6D)
MKMDDKTIV

SEQ ID NO: 20 - protein, 9 aa, Arabidopsis thaliana
  REGION 1..9: note = Description of sequence: Peptide, name: CRY2(K6E)
MKMDKKTIV